(19) United States
(12) Patent Application Publication
(10) Pub. No.: US 2011/0098284 A1
(43) Pub. Date: Apr. 28, 2011

(54) Methods and compositions for treating pain

(76) Inventor: Najib Babul, Blue Bell, PA (US)

(21) Appl. No.: 11/919,306

(22) PCT Filed: Apr. 28, 2006
(86) PCT No.: PCT/US2006/016078

§ 371 Date: Jun. 19, 2008

Related U.S. Application Data
(60) Provisional application No. 60/675,442, filed on Apr. 28, 2005.

(51) Int. Cl. A61K 31/542; A61K 31/192; A61K 31/5415; A61K 31/132; A61P 25/00; A61P 29/00

(52) U.S. Cl. 514/226.5; 514/570; 514/673

(57) ABSTRACT

The present invention provides a method of treating or preventing pain, inflammation or fever comprising administering to a subject in need of such treatment or prevention a therapeutically effective amount of one or more selected NSAIDs, in racemic, enantiomeric excess, or enantiomeric form, in combination with ethylenediamine and/or piperazine.

The application claims the benefit of U.S. Provisional Application No. 60/675,442, filed Apr. 28, 2005, which is herein incorporated by reference in its entirety.

### BACKGROUND OF THE INVENTION

1. Field of the Invention

The present invention is in the field of pharmaceutical compositions and the use thereof for treating and preventing pain, inflammation and fever.

2. Background Art

The medical condition of pain is a complex physiological process that involves a number of sensory and neural mechanisms. Pain can be defined as an unpleasant sensory or emotional experience associated with actual or potential tissue damage, or described in terms of such damage.

Pain is most often classified by time course or mechanism as acute pain, inflammatory pain, visceral pain, breakthrough pain, nociceptive pain, neuropathic pain, chronic pain, or cancer-related pain. Acute pain is a normal, predictable physiological response to an adverse chemical, thermal, or mechanical stimulus. Acute pain is normally self-limiting. When the condition producing the pain resolves, the pain goes away. Chronic pain is usually defined as pain persisting longer than the expected time of tissue healing. Chronic pain includes such syndromes as low back pain, myofascial pain, osteoarthitis, cancer pain, neuropathic pain, fibromyalgia, and inflammatory pain states such as rheumatoid arthritis.

Acute pain is usually a consequence of an identifiable insult, such as surgery or other trauma, or a consequence of a disease, e.g., kidney stones, mechanical low back pain, etc. According to public health statistics, several hundred million people worldwide undergo inpatient or outpatient surgery each year. In addition, several hundred million visits are made annually to the emergency room. Of these emergency room visits, it is estimated by survey data that more than 20% require analgesic treatment. Recent studies have shown that more than 60% of patients who undergo surgery experience moderate to severe pain despite analgesic treatment.

Currently, medical practitioners may choose from several well-accepted classes of pharmaceutical agents in their attempts to alleviate pain. Acute pain is managed with a variety of drugs, frequently in combination, including opioid analgesics, e.g., morphine, hydromorphone, hydrocodone, oxycodone, tramadol, and codeine; acetaminophen; non-steroidal anti-inflammatory drugs (NSAIDs) e.g., ketoprofen, ibuprofen, naproxen, tiaprofenic acid, aceclofenac, diclofenac, piroxicam, loxaprofen, fenoprofen, flurbiprofen, tenoxicam, lornoxicam, acetylsalicylic acid, flufenamic acid, mefenamic acid, niflumic acid, tolfenamic acid, diflunisal, etodolac, fenbufen, isoxicam, pirprofen, sulindac, tolmetin, and piketoprofen and more recently, cyclo-oxygenase isoform 2 (COX-2) selective NSAIDs, e.g., celecoxib, valdecoxib, piketoprofen, etoricoxib, rofecoxib, and lumiracoxib.

Treatment of acute pain is usually with the oral route of administration. However, parenteral drug formulations have become a very important component in the arsenal of available drug delivery options, particularly for drugs having analgesic, anti-inflammatory or antipyretic effects. Parenteral routes of administration, including subcutaneous, intramuscular, intrathecal, epidural and intravenous injection, offer numerous benefits over oral delivery in particular situations, for a wide variety of drugs. For example, parenteral administration of a drug typically results in attainment of a therapeutically effective blood concentration of the drug in a shorter time than is achievable by oral administration. This is especially true of intravenous injection, whereby the drug is placed directly into the bloodstream. Parenteral administration can also result in more predictable blood serum concentrations of a drug, because drug loss in, the gastrointestinal tract due to absorption, distribution, metabolism, binding to food and other causes are eliminated. Parenteral administration is generally the preferred method of drug delivery in emergency situations, and is also useful in treating subjects who are uncooperative, unconscious, or otherwise unable or unwilling to accept oral medication.

Parenteral drugs are particularly useful for treating a condition such as pain, inflammation or fever when: 1) the condition is of severe intensity; 2) there is a need for rapid onset of effect; 3) there is a need for rapid or frequent dose titration to keep condition under control; 4) the patient is unable to receive oral medication e.g., due to nausea, vomiting, confusion, obtundation, loss of consciousness and bowel obstruction.

Of the many challenges that occur when pharmacologically treating any disease or pathological condition, including pain, fever and inflammation, alleviating the symptoms without causing counterproductive side effects is often the greatest. This challenge presents itself when medical practitioners use medicinal agents to treat pain, fever, and inflammation. Although the aforementioned pharmacological classes are frequently effective for the treatment of certain types of pain, fever, and/or inflammation, use of these analgesic agents produces a number of significant undesirable aide effects.

The main mechanism by which opioids exert an analgesic effect is through agonism at the various opioid receptors, e.g., mu, delta and/or kappa. The opioids are well-known for their potential for physical dependence and addiction. Other side effects of opioids, particularly in the acute setting and more particularly in non-opioid tolerant or opioid naïve patients, include nausea, vomiting, pruritus, constipation, sedation, and potentially fatal respiratory depression. When a subject is tolerant to opioids, increased doses are required to achieve a satisfactory analgesic effect. For this reason, alternative therapies for the management of acute pain are widely sought, so as to minimize the amount of opioid patients will require for pain management. Compounds which serve as replacements for opioids or reduce the required opioid dose (opioid sparing) have utility in the treatment of pain.

The NSAIDs, as a class, are highly effective as analgesics. They are used to treat both acute and chronic pain, usually by the oral route of administration. The main mechanism by which NSAIDs exert an analgesic effect is through the inhibition of the synthesis of certain prostaglandin, or prostanoids. The synthesis of prostanoids utilizes two distinct COX enzymes: COX-1 and COX-2. Traditional NSAIDs inhibit both enzymes. NSAIDs may also inhibit other lipogenic enzymes, such as 5-lipooxygenase. Although NSAIDs are not addictive, they are not without significant toxic effects, such as gastrointestinal injury, hepatotoxicity and decrease clotting ability.

Ketorolac is presently the only NSAID available in parenteral form in the United States. However, the clinical utility of parenteral ketorolac is limited due to its adverse effect profile and a restriction on duration of use. In a number of countries, including France, Germany, The Netherlands, Portugal, and Greece, injectable ketorolac has been withdrawn from the market by the local health authorities, due to its high propensity to cause serious and life threatening side effects. Injectable ketorolac must be given every 4 to 6 hours to provide continuous relief of pain. In some countries, other parenteral NSAIDs such as diclofenac and lomoxicam are also available, but in most countries, ketorolac still remains by far the most frequently used injectable NSA/D.

Parecoxib, a parenteral COX-2 selective NSAID, has been approved in Europe with restrictions on its use and has been twice denied approval by the United States Food and Drug Administration for the management of acute post-surgical pain. Although parecoxib (the injectable prodrug of the now withdrawn drug, valdecoxib) is an effective analgesic, there remain serious unanswered questions about the safety of short-term use in the postsurgical setting. An important early adverse postsurgical safety signal came from a CABG study included in the original U.S. NDA. In the parecoxib and valdecoxib group, 19.0% had serious adverse events, versus 9.9% in the placebo group. Citing deficiencies in the data, including a numerically higher incidence of myocardial infarctions (1.9% vs. 0.7%) and cerebrovascular events (2.6% vs. 0.7%) and deaths (4 vs. 0), parecoxib received a non-approvable letter from the FDA in 2001. FDA concluded that “the adverse event profile of parecoxib was generally worse than that of placebo in this trial. Although not statistically significantly different, the number of deaths, myocardial infarctions, cerebrovascular accidents, pulmonary embolisms, along with renal and pulmonary complications were also numerically more frequent for parecoxib during this IV dosing period than placebo. In fact, during the entire study period, the incidence of these clinically relevant adverse events associated with parecoxib/valdecoxib was statistically significantly different than placebo. Similarly, during the entire study period, more patients in the parecoxib/valdecoxib versus the placebo group withdrew from the study due to an adverse event.” (FDA Medical Officer Review: Parecoxib NDA 21-294). Follow-up studies conducted with parecoxib have raised additional safety issues. In one trial, cardiovascular events (including myocardial infarction, cardiac arrest, stroke, and pulmonary embolism) occurred significantly more frequently in the parecoxib and valdecoxib group than on placebo (2.0 percent vs. 0.5 percent; P=0.03). In another trial, there were significantly more sternal wound infections on parecoxib than placebo (3.2% vs. 0%; P=0.03). (See Nussmeier et al., N. Engl. J. Med. 352:1081-91 (2005); Ott et al., J. Thorac. Cardiovasc. Surg. 125:1481-92 (2003) Babul et al, Anesthesia and Analgesia 2006; 102:644-56; Babul et al., Anesthesiology 2006; 104:375). In 2002, the European Medicines Evaluation Agency (EMEA) issued a public statement on parecoxib concerning the risk of serious hypersensitivity and skin reactions, including Stevens-Johnson syndrome, toxic epidermal necrolysis, erythema multiforme, and exfoliative dermatitis as well as anaphylaxis and angioedema. The EMEA has since contraindicated the use parecoxib in patients with ischemic heart disease and stroke. Excluding individuals with silent ischemia, this translates to approximately 20 million at risk patients in the United States. For this reason, it has been recommended that non-selective NSAIDs may be a “better choice” than COX-2 selective agents in patients with cardiovascular disease. (See Stiller & Hjemdahl, J. Hypertension 21:1615-18 (2003)).

In view of the safety limitations of parenteral ketorolac and parenteral parecoxib, there is a need for safer, alternative parenteral NSAIDs.

When administered by the IV route, many NSAIDs are associated with one or more drawbacks, including poor solubility, pain on injection, venous irritation, venous phlebitis, intramuscular pain and irritation, and the need to inject in a large volume of physiologic fluid and/or as a slow IV infusion (instead of as a bolus) to minimize local irritation. For example, Kostamovaara et al. (Br. J. Anaesth. 81:369-372 (1998)) found that after hip replacement surgery, 14% of patients receiving diclofenac 75 mg IV over 30 minutes followed by diclofenac 75 mg/15.5 hours and 10% of patients receiving ketoprofen 100 mg bolus over 30 min, followed by ketoprofen 100 mg/15.5 hours developed phlebitis. Campbell and Watters, Br. J. Anaesth. 62:545-547 (1989), evaluated patients after minor orthopedic, plastic, and general surgery who had received diclofenac 1 mg/kg over 10 min as a solution of 25 mg/mL or 5 mg/mL and found a higher incidence of phlebitis (hand, 85% & arm, 58%) in patients receiving 25 mg/mL compared with 5 mg/mL (p=0.02). The incidence of thrombosis was reduced markedly in patients receiving diclofenac IV using a diluted infusion (38% hand veins; 8% arm veins).

In a further attempt to prevent thrombosis, Gopinath, Br. J. Anaesth. 67:803 (1991), diluted diclofenac in 100 to 200 mL of physiological fluid and infused it over 15 to 20 minutes using a fresh vein. Only delayed venous thrombosis (>72 hours) was observed, suggesting the value of diluted diclofenac infused over a longer period of time as a method for reducing venous sequelae of IV diclofenac. Morrow et al., Anaesth. 48:585-87 (1993), reported after diclofenac 75 mg or ketorolac 30 mg IM, the incidence of injection site pain was 31% and 3%, respectively. Claeys et al., Acta Anaesth. Scand. 36:270-275 (1992), reported that after major orthopedic surgery, 5% of patients receiving diclofenac 0.35 mg/kg IV, followed by diclofenac 0.90 mcg/min as a continuous IV infusion experienced phlebitis 6 hours after start of treatment. Tarkkila, et al., Can. J. Anesth. 43:216-20 (1996), administered diclofenac 1 mg/kg in 100 mL saline every 12 hours×2 doses to patients undergoing maxillofacial surgery and found that 10% of patients developed phlebitis. Pillans and O'Connor, Ann. Pharmacother. 29:264-6 (1995), reported six cases of severe local reactions associated with IM diclofenac. Three patients developed extensive tissue necrosis at the IM injection site. Necrotizing fasciitis in an additional three patients was associated with complications life threatening complications such as adult respiratory distress syndrome, renal failure, shock, and disseminated intravascular coagulation. Two of the six patients died. Rygnestad and Kvam, Acta Anaesthesia Scand 39:1128-30 (1995), described extensive muscle necrosis and a fatal case of streptococcal myositis 48 hours after an IM injection of diclofenac to one patient.

In some European countries, a formulation of injectable diclofenac is approved as a 75 mg/3 mL solution for IM administration. It contains benzyl alcohol as a solubilizing agent. Benzyl alcohol has been associated with a fatal toxic syndrome in premature infants, and parenteral preparations containing the benzyl alcohol are not recommended in neonates. (See British National Formulary, No. 49, March 2005). The 3 mL volume for IM administration is more that the 2 mL maximum recommended volume and therefore it may be associated with increased frequency and intensity of injection site pain. Some regulatory health authorities have required the following dosing language for IM diclofenac acid (Voltarol Ampoules. U.K. Summary of Product Characteristics, March 2005):
“The following directions for intramuscular injection must be adhered to in order to avoid damage to a nerve or other tissue at the injection site. One ampoule once (or in severe cases twice) daily intramuscularly by deep intragluteal injection into the upper outer quadrant. If two injections daily are required it is advised that the alternative buttock be used for the second injection. Alternatively, one ampoule of 75 mg can be combined with other dosage forms of diclofenac (tablets or suppositories) up to the maximum daily dosage of 15 mg.”

A different 75 mg/3 mL formulation of diclofenac is approved for intravenous use (the IM formulation is contraindicated for intravenous use) in some countries with the following language (Voltaren Injection Data Sheet, New Zealand, Sep. 7, 1999):
“Diclofenac must not be given as an intravenous bolus injection. Diclofenac must be diluted with 100-500 mL of either sodium chloride solution (0.9%) or glucose solution (5%). Both solutions should be buffered with sodium bicarbonate solution (0.5 mL 8.4% or 1 mL 4.2%). Only clear solutions should be used. Two alternative regimens are recommended. For the treatment of moderate to severe post-operative pain, 75 mg should be infused continuously over a period of 30 minutes to 2 hours. If necessary, treatment may be repeated after 4-6 hours, not exceeding 150 mg within any period of 24 hours. For the prevention of post-operative pain, a loading dose of 25 to 50 mg should be infused after surgery over 15 minutes to 1 hour, followed by a continuous infusion of approximately 5 mg per hour up to a maximum daily dosage of 150 mg.”

In other countries, such as South Africa, as is the case with ketoprofen, diclofenac solubilized with benzyl alcohol is strictly prohibited for any IV use. (See ACU-Diclofenac Injection Package Insert, Mar. 23, 1993 & Q-Med Diclofenac Injection Package Insert, Apr. 29, 1996).

A review of injection related complications over the period 1992 to 2003 in New Zealand revealed that 22.6% of all accepted claims for injection related complications were due to IM administration of diclofenac, resulting in inflammation, protracted pain, abscesses And nerve injury (Matthews R. Medical Misadventure Unit, January & February, 2003, New Zealand).

Pain as a consequence of administration of a drug intended to ameliorate other pains can lead to patient refusal to accept therapy and require diagnostic workup to rule out infiltration, extravasation and infection at the injection site. It can also lead to further medical complications, increased reliance on opioid analgesics for pain relief, delayed discharge from hospital and increased cost of therapy. Patients report the pain sensation as stinging, burning, soreness, tenderness, aching, throbbing, cramping, gripping and radiating. It may be localized to the injection site or it may radiate to the proximal area, e.g., the arm. It may or may not be accompanied by redness.

Among the contributing factors to venous irritation and injection site pain are the intrinsic irritating properties of the drug, the duration and frequency of IV infusion, the pH and osmolarity of the infusate, catheter placement techniques, the health of the patient's veins, and the likelihood of precipitation of drug upon contact with blood. This phenomenon is frequently referred to as peripheral vein infusion thrombophlebitis, or phlebitis, and it is one of the most common complications of IV therapy. Phlebitis can result from mechanical irritation, chemical irritation, or as a pharmacological response by the vein wall cells to the drug.

Phlebitis is an inflammation of the vein in which endothelial cells of the venous wall become irritated and cells roughen, allowing the adherence of platelets. The site is tender to touch and can be very painful. Phlebitis can prolong hospitalization unless it is treated early. The process of phlebitis formation involves increased capillary permeability, resulting in leakage of proteins and fluids into interstitial space. The traumatized tissue continues to be irritated chemically. This in turn provokes an immune reaction, resulting in mobilization of leukocytes release of inflammatory mediators [Phillips, L D, Manual of I.V. Therapeutics, 3^{rd} Edition (2001), p. 352-61, FA Davis Co., Philadelphia; Weinstein S. M., Plumer's Principles & Practice of Intravenous Therapy, 7^{th} Edition (2001), p. 149-77, Lippincott, Philadelphia; Josephson D L, Intravenous Infusion therapy for Nurses, 2^{nd} Edition (2004), p. 92-119, Thomson Delmar Learning, New York].

Intravenous administration of NSAIDs in racemic or enantiomeric form, including ketoprofen, ibuprofen, naproxen, tiaprofenic acid, aceclofenac, diclofenac, piroxicam, loxaprofen, fenoprofen, flurbiprofen, tenoxicam, lomoxicam, acetylsalicylic acid, flufenamic acid, mefenamic acid, niflumic acid, tolfenamic acid, diflunisal, etodolac, fenbufen, isoxicam, pirprofen, sulindac, tolmetin and piketoprofen can produce phlebitis. Phlebitis occurs when a vein becomes inflamed by irritation or vesicant solutions or drugs. Although the use of a 0.5 to 1.0 micron inline filter can remove particulate matter not visible to the naked eye, it cannot eliminate the precipitation that occurs when the drug comes in contact with blood. Precipitation is considered to be a major cause of injection site pain, venous irritation and phlebitis.

In the case of mechanical irritation, precipitated molecules can be irregular or needle shaped and can act like sandpaper, scraping the vein walls as they are carried through the blood stream. Even relatively small quantities of particulate matter in infusate can cause phlebitis. For this reason, many IV fluids are passed through an in-line filter before injection. However, pre-injection filtration is not effective in cases where the offending particulate matter is created by dilution of the formulation in the bloodstream.

Chemical irritation results from direct contact of irritants with the vein wall cells. Solvents, acids and bases, and some drugs can damage living tissue. In addition, some dissolved drugs can produce undesirable pharmacological effects by reacting with specific receptors on the vein walls. The undesirable effects caused pharmacologically and chemically are worsened by direct cellular contact with the highly concentrated precipitate. As a result, regardless of the cause, precipitation of the drug within the vein upon dilution significantly exacerbates the irritation and is a key factor in determining the duration and severity of the irritation.

A commonly used in vivo phlebitis screening model involves injecting the prototype formulation into the marginal ear vein of the rabbit. Visual observation at the injection site are used to evaluate the degree of phlebitis. Symptoms of phlebitis range from mild erythema and edema to severe necrosis. Since the visual signs of phlebitis may develop slowly, the rabbits are observed over a period of 24 hours. A major disadvantage of the in vivo model is the cost and effort of studies in animals.

A non-animal model for evaluating precipitation-induced phlebitis, capable of assessing mechanical phlebitis has previously been developed and statistically validated. (See Yalkowsky, S H et al., J. Pharm. Sci. 72:1014-19 (1983); Johnson et al., J. Pharm. Sci. 92:1574-81 (2003)). This in vitro procedure for mixing a parenteral formulation with isotonic Sorensen's phosphate buffer (ISPB) at pH 7.4 in a dynamic experiment simulates its injection into a vein and detects precipitation. The resulting precipitate, if any, creates a haziness that is quantitated turbidimetrically on an ultraviolet spectrophotometer downstream from the mixing (injection) site.

Modifications of this technique were shown by Davio et al., Pharm. Res. 8:80-83 (1991), to provide results that correlate well with monkey data. The researchers note that they would not have expected the drug, ditekiren, to precipitate in vitro based on equilibrium solubility measurements at physiological temperature and pH. However, significant drug emboli were observed in the lungs and heart of monkeys used in the in vivo study. When a dynamic injection in vitro model, similar to that noted above was implemented, accurate in vivo prediction of precipitation was obtained. Greenfield et al., Pharm. Res. 8:475-79 (1991), have also evaluated ditekiren for prediction of in vivo precipitation. In the static assessment method, plasma from both monkey and humans were used to dilute the formulation in acidified glucose. These experiments suggested an absence of significant precipitation upon dilution of ditekiren, formulated at concentrations 25 times in excess of that anticipated for clinical studies. Thus, the static method failed to predict the in vivo precipitation, whereas the dynamic apparatus succeeded.

Reference may be made to U.S. Pat. No. 5,554,789 which describes a tromethamine salt of the dextroenantiomer of ketoprofen, i.e., dexketoprofen. This salt is said to be more soluble than the free acid of dexketoprofen.

It has been reported that the complexation of the very slightly soluble NSAID ibuprofen ((±)-2-(4-isobutylphenyl)propionic acid) with beta-hydroxypropylcyclodextrin and subsequent lyophilization sufficiently increases the solubility of the complex to allow for IV administration of a 200 mg dose in 8.3 mL of water, packaged in a 20 mL vial. The art discloses that increasing the solubility of ibuprofen through formation of inclusion complexes with cyclodextrins may allow for its administration by the IV route, thereby preventing or reducing the gastrointestinal toxicity through avoidance of the oral route. (See Kagkadis, K A et al., PDA Journal of Pharmaceutical Science and Technology 5:317-23 (1996)).

Reference may be made to U.S. Pat. No. 5,679,660, which describes a method of preparing an injectable pharmaceutical or veterinary composition of diclofenac or its pharmaceutically acceptable salt and 2-hydroxypropyl-β-cyclodextrin or an inclusion complex of same.

It has been reported that the solubility of the arylpropionic acid NSAID fenoprofen for oral administration can be enhanced by salification with calcium, potassium, sodium, magnesium and ammonium, with only the calcium salt providing very good stability against humidity (Kai E, et al., Pharmazie 1:30-32 (1987)).

It has been reported that IM administration of lyophilized ketoprofen lysinate at dose of 2.2 mg/kg to horses was well tolerated, with a slow rate of absorption (mean residence time=129 minutes), but with a high (93%) absolute bioavailability. (See Anfossi, P., et al., Vet. Quart. 19:65-68 (1997)).

It has been reported that that the aqueous solubility of ketoprofen can be increased with the use of ethanol, polyethylene glycol (PEG)-600, PEG-400, PEG-200, propylene glycol and glycerol as cosolvents. (See Singhai, et al., Pharmazie 51:737-740 (1996); Singhai, et al., Pharmazie 52:149-51 (1997)).

It has been reported that that the aqueous solubility of flurbiprofen can be increased by use of the hydrotropes, sodium benzoate, sodium hydroxybenzoate and methyl-p-hydroxybenzoate sodium. In each formulation, 0.1% w/v sodium metabisulfite and 0.01% ethylenediamine-tetraacetic acid (EDTA) were added as antioxidant and chelating agents, respectively to provide a 100 mg dose in a final volume of greater than 4 mL. (See Gupta, et al., Pharmazie 52:709-12 (1997)). This formulation has many of the aforementioned drawbacks. In any case, the art does not teach about use of ethylenediamine or piperazine, nor is there suggestion of or specific reference to a reduced risk of precipitation-induced phlebitis.

It has been reported that that the aqueous solubility and intrinsic dissolution rate of ibuprofen and ketoprofen are increased when mixed with N-methylglucamine. (See de Villiers, et al., Drug Dev. Ind. Pharm. 8:967-72 (1999)).

Reference may also be made to U.S. Pat. No. 4,748,174, which discloses an acid addition salt of N-methylglucamine with certain NSAIDs for parenteral, oral, rectal, and topical administration.

Reference may also be made to U.S. Pat. No. 5,028,625, which discloses an acid addition salt of N-methylglucamine with ibuprofen allegedly having improved solubility, taste, and palatability for an oral chewable tablet.

Reference may also be made to U.S. Pat. No. 4,279,926 which discloses salification among the salts of phenylalkanoic acids, ibuprofen or (±)-2-(4-isobutylphenyl)propionic acid with arginine and lysine. Compositions are prepared which are suitable for parenteral administration and include sterile aqueous or non-aqueous solutions, suspensions, or emulsions. The only aqueous composition suitable for parenteral administration disclosed in this patent contains 3 mL of 95% aqueous ethanol and 500 mg of ibuprofen, to produce soluble salts which can more readily be administered by the parenteral route, thereby preventing or reducing the gastrointestinal toxicity. Although the salts are reported to be well tolerated by the IM route, they exceed the 2 mL maximum recommended volume for IM injection at most medical institutions and their ethanol content would be not at all suitable to administer intravenously.

Reference may also be made to U.S. Pat. No. 5,895,789, which discloses alkylammonium salts, specifically lysine, tromethamine, dropropizine, and 3-(4-phenyl-1-piperazinyl)-1,2-propanediols, of 2-arylpropionic acids, for parenteral administration, 1) chosen from the group consisting of ketoprofen, ibuprofen, naproxen and tiaprofenic acid and 2) having osmolarity between 270 and 310 mOsm/kg and 3) having a pH between 7.0 and 7.5 and 4) being free of preservatives and supporting substances and 5) being prepared and kept in inert gas and 6) stored away from light in dark glass containers. Purportedly, the said pharmaceutical compositions are more tolerable than compositions for parenteral use on the market containing the same NSAIDs as acids.

Reference may also be made to U.S. Pat. No. 6,342,530 B1, which discloses a lysine salt of ibuprofen dissolved in sterile water for parenteral administration to form a solution in the absence of an inert atmosphere and substantially free of any excipients, organic solvent, buffer, acid, base, other salts and capable of storage in the absence of an inert atmosphere.

U.S. Pat. No. 5,510,385 discloses lysine salts of ibuprofen in a solid form suitable for oral administration such as tablets, caplets, powders and granulates. It is suggested that, when given orally, the lysine salt of ibuprofen has a faster onset of action than ibuprofen acid.

Reference may be made to U.S. Pat. No. 6,069,172, which describes the enantiomerically pure and analgesically inactive salts of (R)-2-(3-benzoylphenyl)propionic acid with achiral and chiral organic bases for the treatment of neutrophil-dependent inflammatory diseases. Aminoalcohols for salification are chosen in the group consisting of ethanolamine, 3-amino-1-propanol, (R)-1-amino-2-propanol, (S)-1-amino-2-propanol, 2-amino-1,3-propandiol, N-(2-hydroxyethyl)pyrrolidine, D-glucamine and L-prolinol, D-glucosamine, and N-methylglucosamine.

Reference may also be made to European Patent No. 0424028 B1, which discloses a crystalline lysinate salt of (S)-(+)-ibuprofen. Purportedly, this invention is based on a faster onset of analgesic action, an enhanced analgesic response and a longer duration of action for (S)-(+)-ibuprofen-L-lysine than the same dose of racemic ibuprofen in the acid form or as a racemic ibuprofen lysinate and a faster onset of analgesic action and enhanced analgesic response compared to (S)-(+)-ibuprofen.

Reference may also be made to U.S. Pat. No. 4,711,906, which discloses solutions of diclofenac for parenteral application which contain a mixture of propylene glycol and polyethylene glycol, preferably with a local anesthetic such as lidocaine.

Reference may be made to U.S. Patent Application No. 2003/019188 and European Patent Application No. EP1129710 which provide for a method of treating photophobia and phonophobia associated with migraine by providing an effective amount of ibuprofen, preferably oral ibuprofen, pharmaceutically acceptable salts thereof, isomers thereof, or mixtures thereof. Examples of suitable pharmaceutically acceptable salts of ibuprofen include any of the inorganic cation salts such as sodium, potassium, lithium, magnesium, calcium, cesium, ammonia, ferrous, zinc, manganous, aluminum, ferric, and manganic; organic salts of ibuprofen with primary, secondary, tertiary and quaternary amines, or mixtures thereof. Examples of such primary, secondary, tertiary and quaternary amines include substituted amines including but not limited to naturally occurring substituted amines, cyclic amines, basic ion exchange resins, and mixtures thereof. More specifically, suitable amines include but are not limited to triethylamine, tripropylamine, 2-dimethylaminoethanol, 2-diethylaminoethanol, lysine, arginine, histidine, caffeine, procaine, N-ethylpiperidine, hydrabamine, choline, betaine, ethylenediamine, glucosamine, tris-(hydroxymethyl)aminomethane, methylglycamine, theobromine, piperazine, piperidine, polyamine resins and the like, and mixtures thereof.

Reference may also be made to U.S. Pat. No. 4,593,044, which discloses salification of diclofenac with L-lysinate in the form of an injectable solution for the treatment of inflammation and rheumatic disorders.

Given the significant therapeutic drawbacks and side effects that accompany the use of NSAIDs for parenteral administration, particularly IV administration, there is a need for new therapeutic methods and pharmaceutical compositions that have analgesic, anti-inflammatory and antipyretic activity. More specifically, there is a need for new analgesic, anti-inflammatory and antipyretic methods and pharmaceutical compositions that are readily soluble and have a rapid onset of effect and robust peak effect, with a reduced incidence and severity of injection site pain, venous irritation and phlebitis.

An ideal parenteral NSAID formulation should provide robust analgesic, anti-inflammatory, and antipyretic effects (rapid onset and high efficacy), as well as good tolerability at and around the IV and IM injection site. Further, an ideal parenteral NSAID formulation should be safe for administration by both the IM and IV routes by avoiding the use of large quantities of potentially toxic solubilizing agents, e.g., benzyl alcohol and by avoiding or minimizing the risk of precipitation following injection.

It is known in the art that if a drug is not soluble in the desired injection volume, it must be solubilized by the addition of one or more water miscible adjuvants. The most common adjuvants are buffers, surfactants and cosolvents, with due consideration of the role of other factors, including pH, osmolality and stability. Unfortunately, drugs solubilized by these means can still precipitate when diluted or injected into the bloodstream. If a therapeutic agent precipitates in a vein, the potential for venous irritation due to mechanical irritation and prolonged local drug exposure at the vein wall, increases greatly. This is an important cause of iatrogenic inflammation of the vein wall, peripheral vein infusion thrombophlebitis (phlebitis), following IV drug administration.

There is reference in the art to various strategies to increase the solubility of NSAIDs, in order to: 1) increase the rate of oral absorption; 2) improve the gastrointestinal tolerability, by rapid oral absorption or use of the parenteral route; and 3) to improve the stability of parenteral dosage forms. However, there is no express mention of strategies to: 1) minimize the risk of phlebitis after IV administration of a NSAID; 2) attain more rapid IV onset of effect (faster peak and higher peak concentrations) by making bolus injections at least as tolerable or preferably more tolerable than currently recommended prolonged N infusions of NSA/Ds; or 3) reduce the cost of N drug administration by delivering a tolerable NSAID dose as direct and rapid N injection, instead of a 15 to 120 minute infusion, with the need for prior dilution and administration through volume control devices. Moreover, for NSAIDs, the art does not teach about the evaluation, testing and selection of candidate salts, complexes, disalts, hydrotopes, buffers, surfactants and cosolvents for precipitation-induced phlebitis, using validated, dynamic experiments that simulate its injection into a vein at physiologic pH or through studies involving surrogate in vivo models of venous irritation.

### BRIEF SUMMARY OF THE INVENTION

A first aspect of the present invention is directed to a method for eliciting a rapid analgesic, anti-inflammatory and antipyretic response after IV or IM injection, said method comprising administering a therapeutically effective amount of the ethylenediamine and/or piperazine complex of a selected NSAID.

A second aspect of the present invention is directed to a method for eliciting a greater peak analgesic, anti-inflammatory, and/or antipyretic response after IV or IM injection, said method comprising administering a therapeutically effective amount of the ethylenediamine and/or piperazine complex of a selected NSAID.

A third aspect of the present invention is directed to a method for improving the intravenous tolerability of an analgesic, anti-inflammatory and antipyretic, e.g., reduced venous irritation, injection site pain, and phlebitis after N bolus injection, short term infusions and continuous infusions, said method comprising administering a therapeutically effective amount of the ethylenediamine and/or piperazine complex of a selected NSAID.

A fourth aspect of the present invention is directed to a novel method for improving the intramuscular tolerability, e.g., reduced injection site pain, muscular irritation, abscesses, nerve irritation and nerve damage after IM injection, e.g., into the gluteus, deltoid, vastus lateralis, said method comprising administering a therapeutically effective amount of the ethylenediamine and/or piperazine complex of the selected NSAID.

A fifth aspect of the present invention is directed to a novel method for improving the tolerability, e.g., reduced injection site pain, subcutaneous irritation, and abscesses after subcutaneous injection, short term subcutaneous infusions and continuous subcutaneous infusions, said method comprising administering a therapeutically effective amount of the ethylenediamine or piperazine complex of the selected NSAID.

A sixth aspect of the present invention is directed to a method for directly administering analgesic, anti-inflammatory and antipyretic treatment by the intravenous route without the need for prior dilution and/or administration through automated or gravity feed volume control devices, e.g., volutrol, minibag, large volume parenteral bag, syringe driver, said method comprising administering a therapeutically effective amount of the ethylenediamine and/or piperazine complex of the selected NSAID.

A seventh aspect of the present invention is directed to a method for safely administering analgesic, antipyretic and anti-inflammatory into soft tissue at the site of, or in close proximity to the pain and inflammation, said method comprising administering a therapeutically effective amount of the ethylenediamine and/or piperazine complex of the NSAID.

An eighth aspect of the present invention is directed to a method for improving the tolerability and safety of intrathecal and epidural administration of an analgesic, anti-inflammatory and antipyretic, e.g., reduced injection site pain and irritation, reduced epidural hematoma and abscess, sterile abscesses, inflammatory mass lesions and intrathecal granulomas after epidural or intrathecal administration, said method comprising administering a therapeutically effective amount of the ethylenediamine and/or piperazine complex of a selected NSAID.

A ninth aspect of the present invention is directed to a method for improving the tolerability and safety of ocular administration of an analgesic or anti-inflammatory, e.g, reduced burning, improved absorption, reduced precipitation, said method comprising administering a therapeutically effective amount of the ethylenediamine and/or piperazine complex of a selected NSAID.

A tenth aspect of the present invention is directed to a composition comprising a selected NSAID; and a compound selected from piperazine and ethylenediamine.

In preferred aspects of the invention, the NSAID is chosen from the group consisting of ketoprofen, dexketoprofen, tenoxicam, and piroxicam. These aspects and additional aspects and embodiments of the present invention are more fully described herein below.

### BRIEF DESCRIPTION OF THE DRAWINGS/FIGURES

FIG. 1 illustrates a simulated plasma NSAID concentration after administration of drug as a rapid IV injection (IV bolus or IV push), as a slow IV infusion over 30 to 120 minutes, and after IM administration.

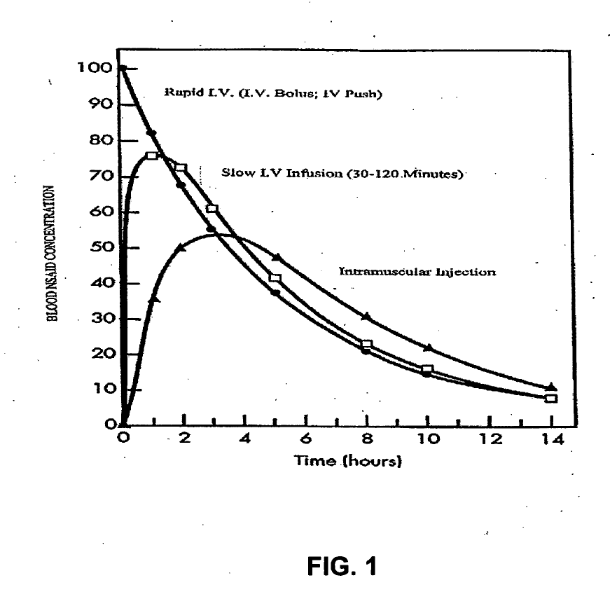

FIG. 2 illustrates the dynamic in vitro apparatus used for predicting mechanical phlebitis. A Harvard Apparatus Precision Syringe Pump 22 was used to direct ISPB (Which is used as a blood surrogate in this experiment), at pH 7.4 and 25 C, through a 40-cm length of flexible Tygon® plastic tubing (type R-3603, inner diameter of 3 mm), then through a Hellma QS quartz flow-through cell with a 1-cm path length at a 5 mL/min flow rate. This flow rate is comparable to that of human blood flow in readily accessible arm veins. ISPB at pH 7.4 was chosen over plasma because it has a buffer capacity of 0.03. A Beckman DU 640 ultraviolet spectrophotometer holding the flow-cell was used to read occlusion of light. The 22-gauge Becton Dickinson Precision Glide sterile stainless steel needle of a 20-mL latex-free Becton Dickinson plastic syringe containing each formulation to be tested was introduced into the flexible tubing at 10 cm upstream from the flow cell. If a formulation precipitates upon injection, it produces opacity in the tubing, which passes through the flow cell and is interpreted by the spectrophotometer as reduced transmittance or increased absorbance.

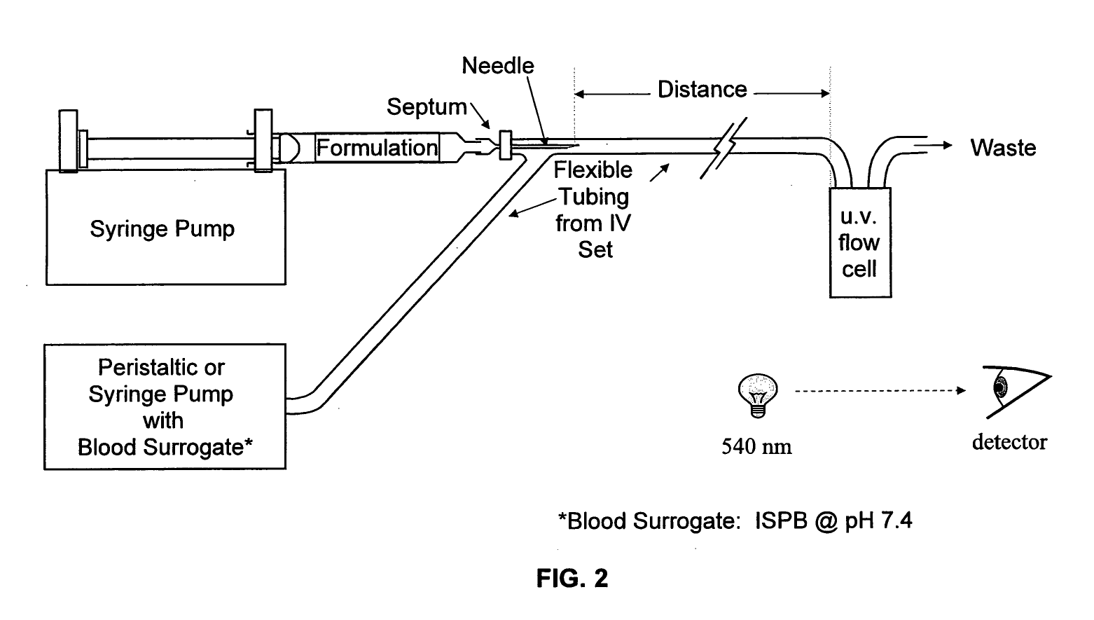

FIG. 3 illustrates a UV wavelength-scan for ketoprofen-ethylenediamine (50 mg/mL) demonstrating absence of absorbance at 540 nm.

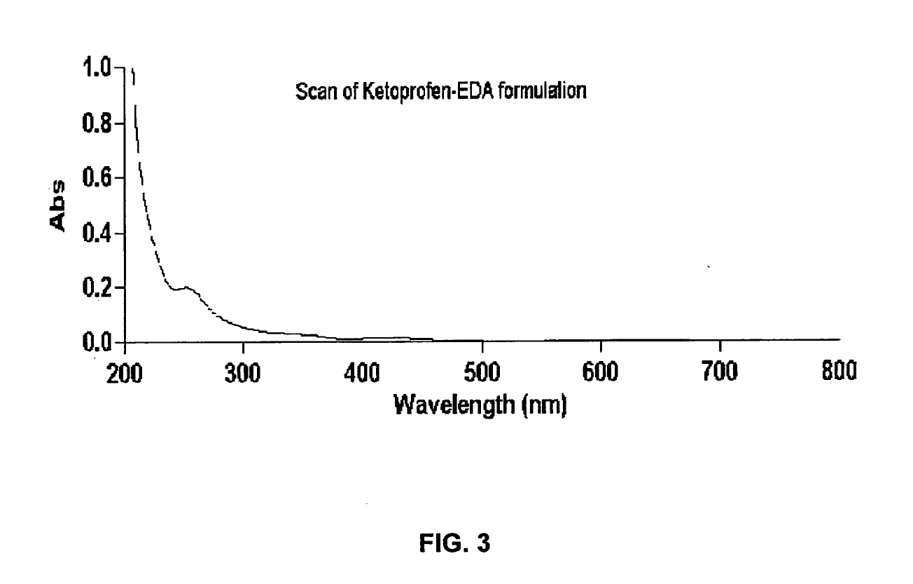

FIG. 4 illustrates dynamic dilution test profile for ketoprofen-ethylenediamine (50 mg/mL) formulation at 540 nm.

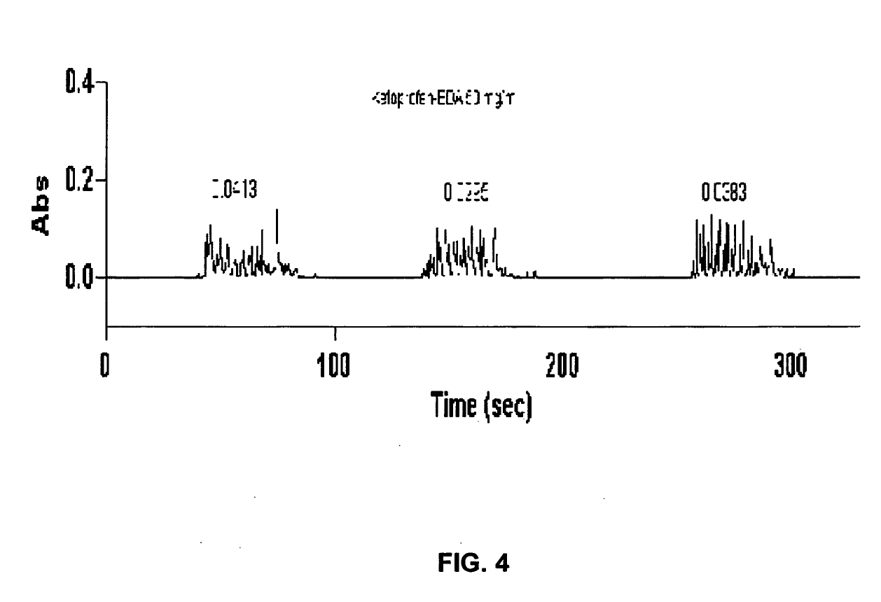

FIG. 5 illustrates a UV wavelength-scan for ketoprofen-piperazine (50 mg/mL) demonstrating absence of absorbance at 540 nm.

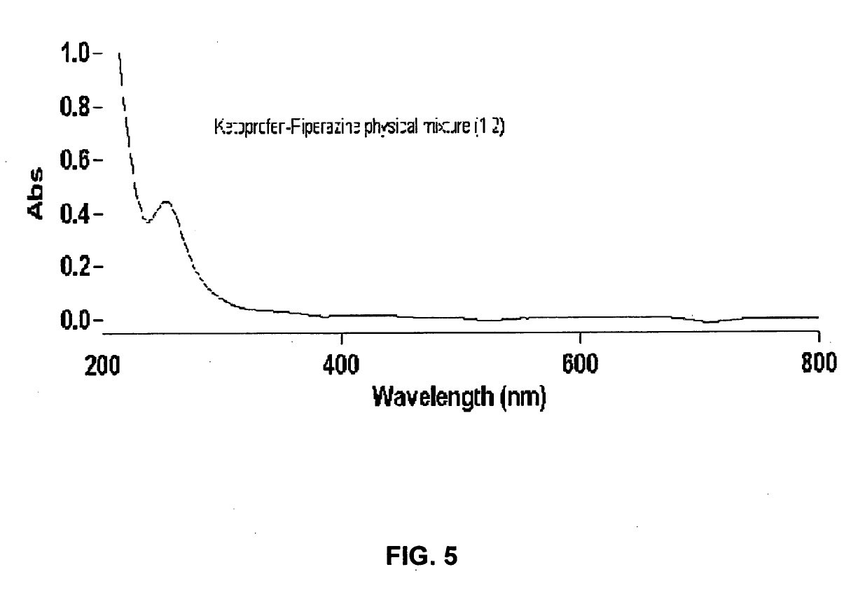

FIG. 6 illustrates dynamic dilution test profile for ketoprofen-piperazine (50 mg/mL) formulation at 540 nm.

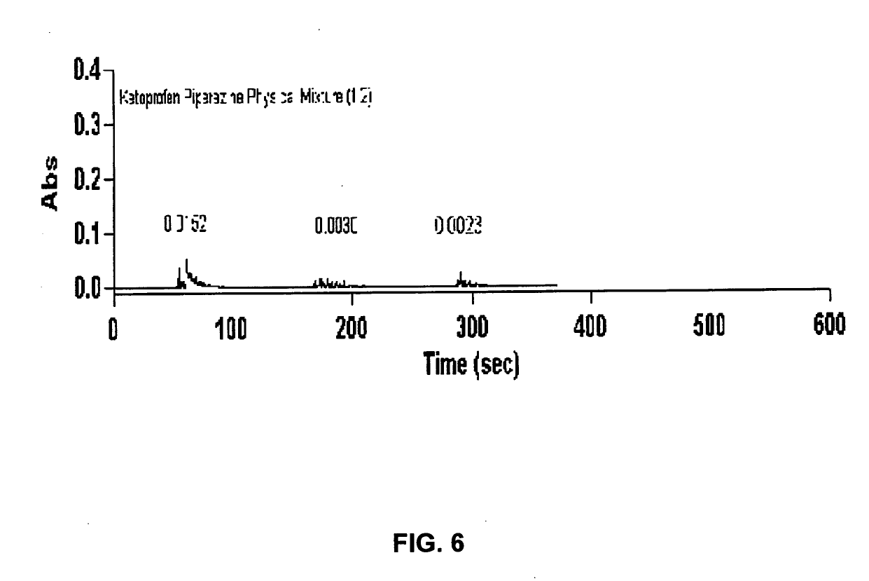

FIG. 7 illustrates a UV wavelength-scan for ketoprofen-piperazine (50 mg/mL) demonstrating absence of absorbance in a 1:1 ratio at 540 nm.

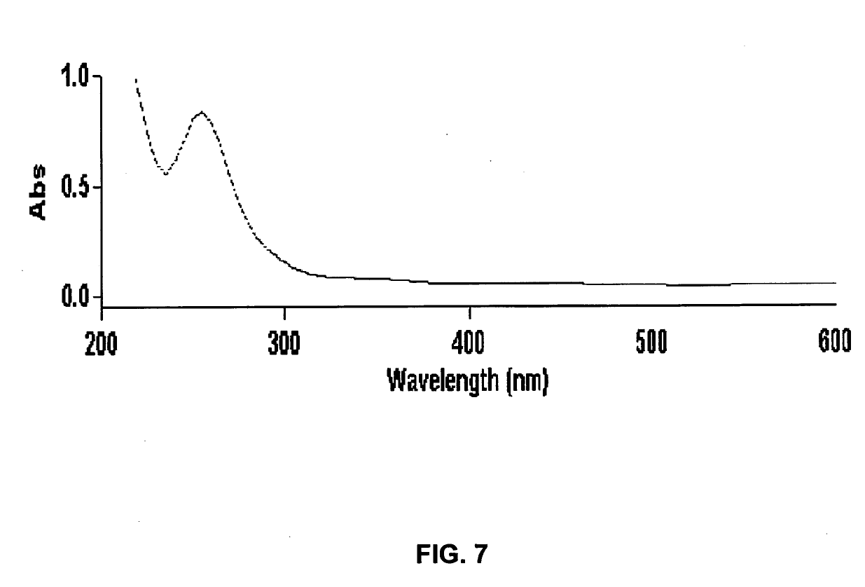

FIG. 8 illustrates dynamic dilution test profile for ketoprofen-piperazine (50 mg/mL) formulation in a 1:1 ratio at 540 nm

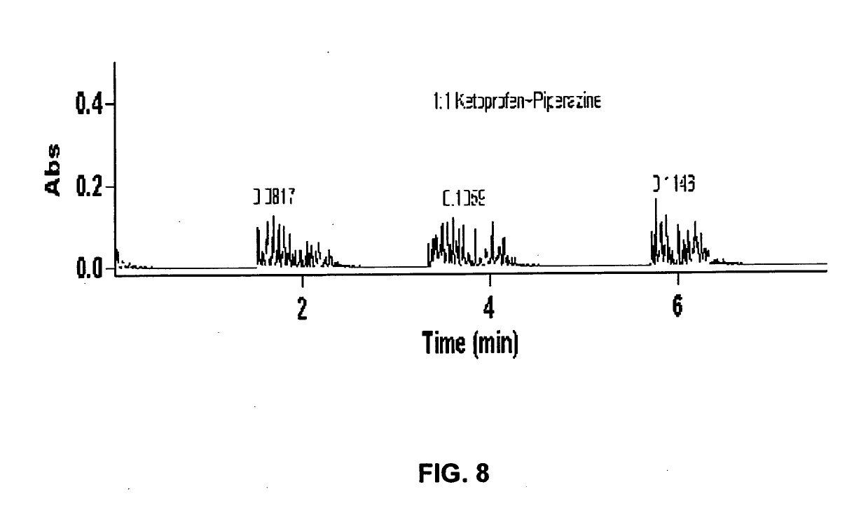

FIG. 9 illustrates a UV wavelength-scan for phenyloin sodium (50 mg/mL), a drug with high phlebitis potential, demonstrating absence of absorbance at 540 nm.

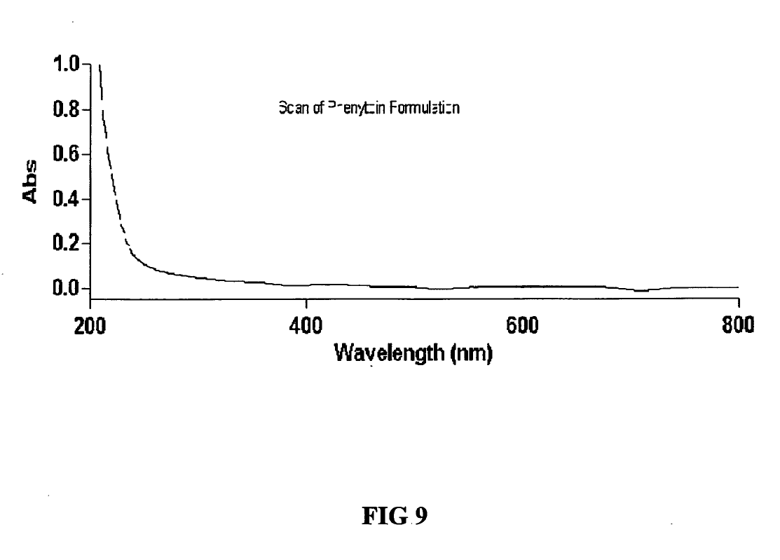

FIG. 10 illustrates dynamic dilution test profile for phenyloin sodium (50 mg/mL). The Y axis is 10 times higher for phenyloin sodium compared to all other formulations.

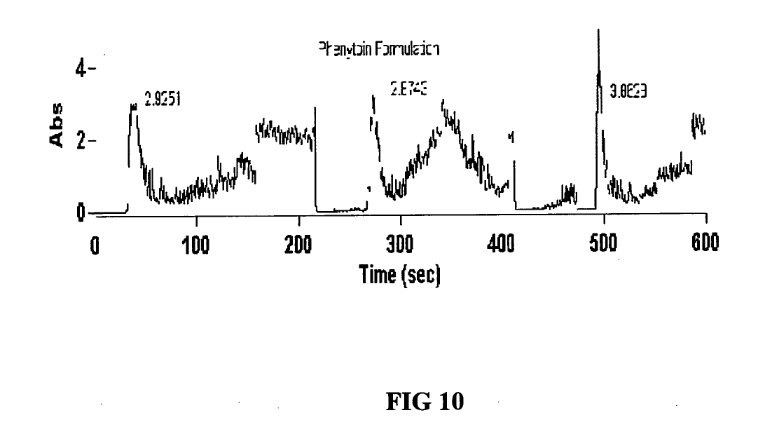

FIG. 11 illustrates dynamic dilution test profile for unsalified ketoprofen suspension (50 mg/mL) adjusted to pH 6.50 and filtered through a 0.45 micron filter before injection at 540 nm, showing approximately 80 and 440-fold greater propensity for precipitation relative to ketoprofen ethylenediamine and ketoprofen piperazine without pH adjustment, respectively.

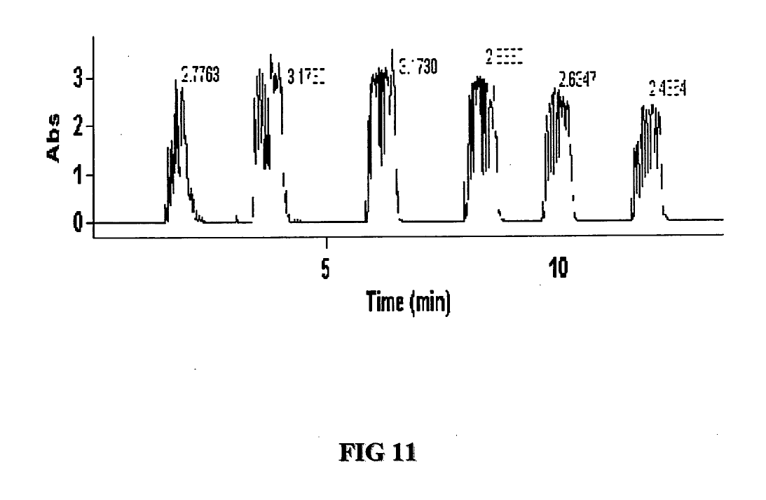

### DETAILED DESCRIPTION OF THE INVENTION

The present invention provides a method of treating or preventing pain, inflammation or fever comprising administering to a subject in need of such treatment or prevention a therapeutically effective amount of one or more selected NSAIDs, in racemic, enantiomeric excess, or enantiomeric form, in combination with ethylenediamine and/or piperazine.

The present invention provides a method of treating or preventing pain, inflammation, or fever comprising administering to a subject in need of such treatment or prevention one or more selected NSAID analgesics, racemic, enantiomeric excess, or enantiomeric form, selected from the group consisting of ketoprofen, ibuprofen, naproxen, tiaprofenic acid, aceclofenac, diclofenac, dexketoprofen, piroxicam, loxaprofen, fenoprofen, flurbiprofen, tenoxicam, lomoxicam, acetylsalicylic acid, flufenamic acid, mefenamic acid, niflumic acid, tolfenamic acid, diflunisal, etodolac, fenbufen, isoxicam, pirprofen, sulindac, tolmetin and piketoprofen, as a complex with ethylenediamine and/or piperazine. Preferably, the NSAID is selected from the group consisting of ketoprofen, dexketoprofen, tenoxicam, and piroxicam. Nonsteroidal anti-inflammatory drugs typically have analgesic, anti-inflammatory, and antipyretic properties. Their mode of action appears to involve inhibition of cyclooxygenases (COX-1 and COX-2), leukotriene biosynthesis, and antibradykinin activity. NSAIDs may be non-selective (inhibit COX-1 and COX-2 isozymes) or COX-2 selective (preferentially inhibit the COX-2 isozymes). Although efficacy with NSAIDs is dose related, there is a “ceiling” to the analgesic effect, i.e., further dose increases do not usually provide a proportional increase in analgesic effect. NSAIDs can produce adverse effects that are usually related to the dose and duration of treatment. Although the exact mechanisms of adverse effects have not been clearly established, at least some appear related to COX-1 inhibition. In addition to their gastrointestinal adverse effects, NSA/Ds produce dose related inhibition of platelet aggregation, prolongation of bleeding time, renal impairment, and hepatotoxicity. Intramuscular and intravenous administration can produce injection site burning and pain.

The phrase “one or more selected NSAID,” as used herein, refers to one or more compounds selected from the group consisting of ketoprofen, ibuprofen, naproxen, tiaprofenic acid, aceclofenac, diclofenac, dexketoprofen, piroxicam, loxaprofen, fenoprofen, flurbiprofen, tenoxicam, lomoxicam, acetylsalicylic acid, flufenamic acid, mefenamic acid, niflumic acid, tolfenamic acid, diflunisal, etodolac, fenbufen, isoxicam, pirprofen, sulindac, tolmetin and piketoprofen. In a preferred embodiment, the phrase “one or more selected NSAID,” as used herein, refers to one or more compounds selected from the group consisting of ketoprofen, dexketoprofen, and piroxicam.

Ketoprofen ((±)-2-(3-benzoylphenyl)propionic acid) or (R,S)-2-(3-benzoylphenyl) propionic acid), is a NSAID with analgesic, anti-inflammatory and antipyretic properties. These properties of ketoprofen have been demonstrated in classical animal models and in vitro test systems. Its mode of action appears to be similar to that of other NSAIDs and includes inhibition of prostaglandin (COX-1 and COX-2 inhibition) and leukotriene biosynthesis, antibradykinin activity, and lysosome membrane-stabilizing activity. Chemically, ketoprofen belongs to the group of substituted 2-phenylpropionic acids.

Ketoprofen as a racemate, or as the analgesically active enantiomer (+)-(S)-2-(3-benzoylphenyl)propionic acid (dexketoprofen), is marketed in a number of countries in a variety of forms, including oral solids, suppositories, and a topical gel. Ketoprofen is soluble in benzene, ethanol, chloroform, acetone, ether, and alkaline solutions, but it is practically insoluble in water.

In a number of countries, an intramuscular (IM) form of ketoprofen is also available for deep intramuscular injection into the gluteus muscle. In countries where the IM solution containing arginine, benzyl alcohol, citric acid, and water for injection is commercially available, its use by the IV route is prohibited due to toxicity of the formulation. IM administration does not provide a very rapid onset of effect, and it is painful, especially with repeated administration or with a large injection volume. Most clinicians and hospitals discourage the repeated IM administration of drugs and the use of IM injection volumes greater than 1 mL. IM injection volumes greater than 2 mL are generally strongly discouraged or prohibited from routine use. Furthermore, IM administration is not appropriate in many patients due to an absence of adequate muscle mass and the possibility of bleeding and hematoma formation, especially if anticoagulated with drugs like heparin or warfarin. IM administration of the acid formulation of NSAIDs has also been associated with sever nerve damage, muscle tissue necrosis, and even death.

A separate IV formulation of racemic ketoprofen is available in a few countries as a lyophilized powder containing sodium hydroxide, glycine, and citric acid. It is recommended by the manufacturer that N ketoprofen be diluted in 100 to 150 mL of 5% dextrose in water or 0.9% saline and administered over approximately 20 minutes. There are numerous disadvantages of this method of administration to a patient with severe acute pain. It requires considerable nursing and pharmacy time and additional material, e.g., N catheter, infusion set, IV sterile solution, swabs, infusion device, and a delay in administering the drug to a patient in severe pain. Furthermore, compared with rapid IV injection, e.g., IV bolus or IV push, an infusion of ketoprofen over 20 minutes means that the onset of analgesia is delayed and the peak pain relief is lower, owing to lower maximal blood concentrations.

Furthermore, N infusion of ketoprofen can cause significant side effects, including venous irritation. (See, e.g., Castagnera, L., et al., Sem Hop Paris 64(32):2179-2182 (1988); Semaine des hospitaux (Paris) 32:2179-88 (1988)).

NSAIDs, including ibuprofen ((2-(4-isobutylphenyl)propionic acid); naproxen (6-methoxy-α-methyl-2-naphthaleneacetic acid); tiaprofenic acid (5-benzoyl-α-methyl-2-thiophenacetic acid); aceclofenac (2-(2,6-dichlorophenylamino)benzeneacetic acid carboxymethylester); diclofenac (2-(2,6-dichlorophenylamino)benzeneacetic acid); piroxicam (4-hydroxy-2-methyl-N-2-pyridinyl-2H-1,2-benzothiazine-3-carboxamide-1,1-dioxide); loxaprofen (alpha-methyl-4-((2-oxocyclopentyl)methyl)benzeneacetic acid); fenoprofen (2-(3-phenoxyphenyl)propionic acid); flurbiprofen (2-fluoro-α-methyl-4-biphenylacetic acid); tenoxicam (4-hydroxy-2-methyl-N-2-pyridinyl-2H-thieno[2,3-e]-1,2-thiazine-3-carboxamide-1,1-dioxide); lomoxicam (6-chloro-4-hydroxy-2-methyl-N-2-pyridyl-2H-thieno-[2,3-e]-1,2-thiazine-3-carboxamide-1,1-dioxide); acetylsalicylic acid (2-acetoxybenzoic acid); flufenamic acid (N-3-(trifluoromethylphenyl)anthranilic acid); mefenamic acid (2-(2,3-dimethylphenyl)aminobenzoic acid); niflumic acid (2-(3-trifluoromethylanilino)nicotinic acid); tolfenamic acid (2-(3-chloro-2-methylphenylamino)benzoic acid); diflunisal (5-(2,4-difluorophenyl) salicylic acid); etodolac (1,8-diethyl-1,3,4,9-tetrahydropyrano[3,4-b]indole-1-acetic acid); fenbufen (g-oxo-(1,1′-biphenyl)-4-butanoic acid); isoxicam (4-hydroxy-2-methyl-N-(5-methyl-3isoxolyl-2H-1,2-benzothiazine-3-carboxamide 1,1-dioxide); pirprofen (2-(3-chloro-4-[3-pyrrolinyl]phenyl)propionic acid); sulindac ((Z)-5-fluoro-2-methyl-1-(p-(methylsulfinyl)-benzylidene)indene-3-acetic acid), tolmetin (1-methyl-5-(p-toluoyl)pyrrole-2-acetic acid); and piketoprofen are associated with one or more of the drawbacks associated with administering the NSAIDs.

It has now been found that ethylenediamine and/or piperazine in combination with one or more selected NSAIDs as defined above, preferably one or more NSAIDs selected from the group consisting of ketoprofen, dexketoprofen, tenoxicam, piroxicam, and mixtures thereof, in racemic, enantiomeric excess, or enantiomeric form, provide an improved method of treating or preventing pain. For example, administrating of a selected NSAID in combination with ethylenediamine and/or piperazine produces, in certain embodiments, a significantly reduced likelihood of venous irritation, injection site pain, and/or phlebitis.

Similarly, it has now been found that ethylenediamine and/or piperazine as the acid addition complex of one or more selected NSAIDs, as defined above, and preferably ketoprofen, dexketoprofen, tenoxicam, and piroxicam, in certain embodiments increase their aqueous solubility and reduce irritation upon parenteral administration. In other embodiments, as explained in some detail below, the methods of the invention provide for administering certain NSAIDs with a significantly reduced likelihood of muscle pain or nerve irritation after IM injection.

In other embodiments, selected NSAIDs, as defined above, and preferably ketoprofen, dexketoprofen, tenoxicam, and piroxicam in racemic, enantiomeric excess, or enantiomeric form, can be advantageously given together with ethylenediamine and/or piperazine, as ethylenediamine and piperazine complexes, preferably as a salt, and administered to animals to not only elicit a more potent analgesic, anti-inflammatory, and/or antipyretic response, but also to evoke such response more rapidly and with a significantly reduced risk of venous irritation, injection site pain and phlebitis, and intramuscular pain. It is understood that the various pharmaceutical compositions described in further detail below may be used in each of the embodiments of the method of the present invention.

In one embodiment of the present invention, the method comprises administering a pharmaceutical composition comprising a selected NSAID and a compound selected from piperazine and ethylenediamine, to a subject in need of rapid analgesic, anti-inflammatory, and/or antipyretic treatment. In preferred embodiments, the pharmaceutical composition is administered intravenously (IV) or intramuscularly (IM).

When given by the intravenous route, the pharmaceutical composition may be administered by rapid IV injection, e.g., IV bolus, IV push, or slow 2 minute IV injection for rapid onset of effect and a more robust peak effect, or alternatively by longer duration infusions. Furthermore, in certain embodiments, when administered by intravenous route, the pharmaceutical composition produces little to substantially no precipitation of the NSAID in the subject's vein.

Alternatively, the pharmaceutical composition can be administered directly into epidural, intrathecal or intra-articular space. For example, the method of this embodiment comprises injecting into an intrathecal space of a subject a composition comprising ketoprofen; piperazine; and a pharmaceutically acceptable carrier, wherein said ketoprofen and piperazine are in a ratio of from about 1:10 to about 10:1, preferably from about 1:2 to about 2:1. Alternatively, the method may comprise injecting into an intra-articular space of a subject a composition comprising ketoprofen; ethylenediamine; and a pharmaceutically acceptable carrier, wherein said ketoprofen and ethylenediamine are in a ratio of from about 1:10 to about 10:1, preferably from about 1:2 to about 2:1.

In certain embodiments, the method of the invention will produce a faster analgesic, anti-inflammatory, and/or antipyretic response of about 5, 10, 20, 30, 40, 50, 60, 70, 80, 90, 100, 120, 140, 150, 160, 180, or 200% compared to the administration of the NSAID alone. The faster response may be measured, for example, at 0.5, 1, 2, 4, 6, 8, 12, or 24 hours post administration.

By way of example, a preferred instance of the present invention is a method of administering a composition comprising ketoprofen and piperazine as an IV injection wherein said administration produces a more rapid onset of action as compared to the administration of ketoprofen alone.

The dosage of the NSAID-piperazine or NSAID-ethylenediamine pharmaceutical composition administered according to this embodiment will vary depending on a number of factors, including but not limited to the type of subject (human or non-human), age, weight, medical history, route of administration, and the like. Exemplary IV dosages include about 2.5 mg to about 600 mg per dose, preferably about 5 mg to about 300 mg per dose. For example, administration according to the present invention, wherein the composition comprises piroxicam-ethylenediamine, ketoprofen-piperazine, or ketoprofen-ethylenediamine, may be given in about 5 to 100 mg of active NSAID per adult dose.

In another embodiment of the present invention, the method comprises administering a pharmaceutical composition comprising a selected NSAID and a compound selected from piperazine, ethylenediamine, and mixtures thereof, to a subject in need of analgesic, anti-inflammatory, and/or antipyretic treatment, wherein said composition is administered IV or IM and wherein a greater peak analgesic, anti-inflammatory, and/or antipyretic response is effected.

In certain embodiments, the method of the invention will produce a greater peak analgesic, anti-inflammatory, and/or antipyretic response of about 5, 10, 20, 30, 40, 50, 60, 70, 80, 90, 100, 120, 140, 150, 160, 180 or 200% compared to the administration of the NSAID alone. The greater peak response may be, for example, measured as of 0.5, 1, 2, 4, 6, 8, 12, or 24 hours post administration.

By way of example, a preferred instance of the present invention is a method of administering a composition comprising ketoprofen and piperazine as an IV injection wherein said administration produces an enhance peak analgesic response. Alternatively, the method may be performed by administering a pharmaceutical composition comprising (a) ketoprofen; and (b) a compound selected from ethylenediamine, piperazine, and mixtures thereof; wherein said composition is substantially free of a surfactant.

For example, the method of this embodiment comprises administering to a human patient via IM injection a composition comprising ketoprofen; piperazine; and a pharmaceutically acceptable carrier, wherein said ketoprofen and piperazine are in a ratio of from about 1:2 to about 2:1. Alternatively, the method may comprise administering to a human patient via IM injection a composition comprising ketoprofen; ethylenediamine; and a pharmaceutically acceptable carrier, wherein said ketoprofen and ethylenediamine are in a ratio of from about 1:2 to about 2:1.

The dosage of the NSAID-piperazine or NSAID-ethylenediamine pharmaceutical composition administered according to this embodiment will vary depending on a number of factors, including but not limited to type of subject (human or non-human), age, weight, medical history, and the like. Exemplary IV dosages include about 2 mg to about 1000 mg per dose, preferably from about 5 mg to about 250 mg per dose. For example, administration according to the present invention, wherein the composition comprises piroxicam-ethylenediamine, ketoprofen-piperazine, or ketoprofen-ethylenediamine, may be given in about 5 to about 150 mg, preferably from about 10 to about 100 mg, of active NSAID per adult dose.

In another embodiment of the present invention, the method comprises administering a pharmaceutical composition comprising a selected NSAID, preferably one or more NSAIDs selected from the group consisting of ketoprofen, dexketoprofen, tenoxicam, piroxicam, and mixtures thereof, and a compound selected from piperazine, ethylenediamine, and mixtures thereof, to a subject in need of analgesic, anti-inflammatory, and/or antipyretic treatment, wherein said composition is administered as an IV bolus injection, short term infusion, or continuous infusion. In preferred embodiments, the method produces reduced venous irritation, reduced injection site pain, and reduced phlebitis after the administration when compared to similar means of administering conventional compositions of the same NSAID.

In certain embodiments, the method of the invention produces improved venous irritation, injection site pain, and/or phlebitis responses by about 5, 10, 20, 30, 40, 50, 60, 70, 80, 90, 100, 120, 140, 150, 180, 200, or 300% compared to the administration of the NSAID alone. In other words, venous irritation, injection site pain, and/or phlebitis responses caused by administration of the NSAID alone will be about 5, 10, 20, 30, 40, 50, 60, 70, 80, 90, 100, 120, 140, 150, 180, 200, or 300% greater compared to the method of the present invention. The reduced venous irritation, injection site pain, and/or phlebitis may be measured, for example, at 1, 2, 4, 8, 12, 24, 48 or 72 hours post administration.

In other instances, the method of the invention obtains a peak pain intensity difference (PPID) or peak pain relief (PPR) for the NSAID, preferably ketoprofen, dexketoprofen, or piroxicam, about 5, 10, 20, 30, 40, 50, 60, 70, 80, 90, 100, 120, 140, 150, 180, 200 or 300% greater than obtained by the administration of the NSAID alone via the same route and with the same amount or absence of venous irritation, injection site pain, and/or phlebitis.

In other instances, the method of the invention obtains an onset of perceptible or meaningful analgesic or antipyretic effect for the NSAID, preferably ketoprofen, dexketoprofen, tenoxicam, or piroxicam, about 5, 10, 20, 30, 40, 50, 60, 70, 80, 90, 100, 120, 140, 150, 180, 200 or 300% faster than obtained by the administration of the NSAID alone via the same route and with the same amount or absence of venous irritation, injection site pain, and/or phlebitis.

In other instances, the method of the invention obtains total pain relief (TOTPAR), sum of pain intensity difference (SPID) or sum of pain relief intensity difference (SPRID) at 0.5, 1, 1.5, 2, 3, 4, 5 or 6 hours post-dose for the NSAID, preferably ketoprofen, dexketoprofen, tenoxicam, or piroxicam, about 5, 10, 20, 30, 40, 50, 60, 70, 80, 90, 100, 120, 140, 150, 180, 200 or 300% greater than obtained by the administration of the NSAID alone via the same route and with the same amount or absence of venous irritation, injection site pain, and/or phlebitis.

By way of example, a preferred instance of the present invention is a method of administering a composition comprising ketoprofen and ethylenediamine as an IV injection wherein said administration produces an enhanced onset, peak or total analgesic response. The amount of venous irritation, reduced injection site pain, and/or reduced phlebitis can be measured using known methods. The reduced venous irritation, reduced injection site pain, and/or reduced phlebitis will lead, in certain instances, to greater patient compliance.

The dosage of the NSAID-piperazine or NSA/D-ethylenediamine pharmaceutical composition administered according to this embodiment will vary depending on a number of factors, including but not limited to type of subject (human or non-human), age, weight, medical history, and the like. Exemplary dosages of this embodiment include about 1 mg to about 600 mg per dose, preferably about 5 mg to about 300 mg per dose. For example, administration according to the present invention, wherein the composition comprises piroxicam-ethylenediamine, ketoprofen-piperazine, or ketoprofen-ethylenediamine, may be given in about 5 to about 100 mg, e.g., about 50 mg, of active NSAID per adult dose.

In another embodiment, the present invention is directed to a method improving the intramuscular tolerability of a selected NSAID, said method comprising administering a pharmaceutical composition comprising a selected NSAID, preferably one or more NSAIDs selected from the group consisting of ketoprofen, dexketoprofen, tenoxicam, piroxicam, and mixtures thereof, and a compound selected from piperazine, ethylenediamine, and mixtures thereof, to a subject in need of analgesic, anti-inflammatory, and/or antipyretic treatment, wherein said composition is administered as an IM injection. In preferred examples, the method provides a means for improving the intramuscular tolerability, e.g., reduced injection site pain, muscular irritation, abscesses, nerve irritation, and nerve damage after IM injection of the NSAID.

In certain embodiments, the method of IM injection produces reduced injection site pain, muscular irritation, abscesses, nerve irritation, and/or nerve damage after IM injection by about 5, 10, 20, 30, 40, 50, 60, 70, 80, 90, 100, 120, 140, 150, 180, 200, or 300% compared to the administration of the NSA/D alone. In other words, injection site pain, muscular irritation, abscesses, nerve irritation, and/or nerve damage responses caused by administration of the NSAID alone will be about 5, 10, 20, 30, 40, 50, 60, 70, 80, 90, 100, 120, 140, 150, 180, 200, or 300% greater compared to the method of the present invention. The reduced injection site pain, muscular irritation, abscesses, nerve irritation, and/or nerve damage may be measured, for example, at 1, 2, 4, 8, 12, 24, 48, 72, 96, 168 or 336 hours post administration.

By way of example, a preferred instance of the present invention is a method of administering a composition comprising ketoprofen and piperazine as an IM injection wherein said administration produces 30% less injection site pain.

The pharmaceutical composition can be administered in varying amounts and volumes. In a preferred embodiment, the IM injection volume of a single therapeutic dose does not exceed about 5 mL, 3 mL, 2 mL, or 1 mL. Thus, for example, the method of the invention may comprise administering, to a subject in need of analgesic, anti-inflammatory, and/or antipyretic treatment, a pharmaceutical composition comprising ketoprofen, piperazine, and saline.

The dosage of the NSAID-piperazine or NSA/D-ethylenediamine pharmaceutical composition administered according to this embodiment will vary depending on a number of factors, including but not limited to type of subject (human or non-human), age, weight, medical history, and the like. Exemplary IM dosages include about 1 mg to about 600 mg per dose, preferably about 5 mg to about 250 mg per dose. For example, administration according to the present invention, wherein the composition comprises piroxicam-ethylenediamine, ketoprofen-piperazine, tenoxicam-ethylenediamine, tenoxicam-piperazine, or ketoprofen-ethylenediamine, may be given in about 5 or about 100 mg of active NSAID per adult dose. Unless otherwise specified, the dosage and concentration amounts recited herein refer to the amount of the NSAID free acid.

In another embodiment, the method of the present invention is directed to improving the tolerability of administering a selected NSAID, wherein said method comprises administering a pharmaceutical composition comprising a selected NSAID, preferably one or more NSAIDs selected from the group consisting of ketoprofen, dexketoprofen, piroxicam, tenoxicam, and mixtures thereof, and a compound selected from piperazine, ethylenediamine, and mixtures thereof, to a subject in need of analgesic, anti-inflammatory, and/or antipyretic treatment, wherein said composition is administered as a subcutaneous injection, short term subcutaneous infusion, or continuous subcutaneous infusion. In preferred embodiments, the method improves tolerability by reducing injection site pain, subcutaneous irritation, and/or abscesses after subcutaneous injection.

In certain embodiments, the method of subcutaneous injection improves tolerability of the NSAID by reducing injection site pain, subcutaneous irritation, and/or abscesses after subcutaneous injection by about 5, 10, 20, 30, 40, 50, 60, 70, 80, 90, 100, 120, 140, 150, 180, 200, or 300% compared to the administration of the NSA/D alone. In other words, injection site pain, subcutaneous irritation, and/or abscesses after subcutaneous injection experienced by administration of the NSA/D alone will be about 5, 10, 20, 30, 40, 50, 60, 70, 80, 90, 100, 120, 140, 150, 180, 200, or 300% greater compared to the method of the present invention. The reduced injection site pain, subcutaneous irritation, and/or abscesses may be measured, for example, at 1, 2, 4, 8, 12, 24, 48, 72, 96, 168, or 336 hours post administration.

By way of example, a preferred instance of the present invention is a method of administering a composition comprising ketoprofen and piperazine as a subcutaneous injection wherein said administration produces 40% less injection site pain.

By way of example, a preferred instance of the present invention is a method of administering a composition comprising piroxicam and ethylenediamine as a subcutaneous injection wherein said administration produces 20% less injection site pain.

The dosage of the NSAID-piperazine or NSAID-ethylenediamine pharmaceutical composition administered according to this embodiment will vary depending on a number of factors, including but not limited to type of subject (human or non-human), age, weight, medical history, and the like. Exemplary subcutaneous dosages include about 1 mg to about 500 mg per dose. For example, administration according to this embodiment of the present invention, wherein the composition comprises piroxicam-ethylenediamine, ketoprofen-piperazine, or ketoprofen-ethylenediamine, may be given in about 5 to 150 mg of active NSAID per adult dose. Alternatively, the pharmaceutical composition comprising the NSAID-piperazine or NSAID-ethylenediamine, e.g., ketoprofen-piperazine or ketoprofen-ethylenediamine, is administered in a range of about 0.05 mg/kg/day to about 30 mg/kg/day, preferably about 0.1 mg/kg/day to about 16 mg/kg/day.

For example, a method of this embodiment comprises administering to a human patient via subcutaneous injection a composition comprising ketoprofen; piperazine; and a pharmaceutically acceptable carrier, wherein said ketoprofen and piperazine are in a ratio of from about 1:3 to about 3:1. Alternatively, the method may comprise administering to a human patient via subcutaneous injection a composition comprising ketoprofen; ethylenediamine; and a pharmaceutically acceptable carrier, wherein said ketoprofen and ethylenediamine are in a ratio of from about 1:3 to about 3:1.

In another embodiment, the present invention is directed to a method of administering an analgesic, anti-inflammatory, and/or antipyretic treatment by the intravenous route without the need for prior dilution and/or administration through automated or gravity feed volume control devices, e.g., volutrol, minibag, large volume parenteral bag, syringe driver, said method comprising administering a therapeutically effective amount of a pharmaceutical composition comprising a selected NSAID, preferably one or more NSAIDs selected from the group consisting of ketoprofen, dexketoprofen, piroxicam, tenoxicam, and mixtures thereof, and a compound selected from piperazine, ethylenediamine, and mixtures thereof.

By way of example, a preferred embodiment of the present invention is a method of administering a, composition comprising ketoprofen and piperazine to a patient in need of analgesic treatment, wherein said composition is through an automated or gravity feed volume control devices, e.g., volutrol, minibag, large volume parenteral bag, and syringe driver.

By way of example, a preferred embodiment of the present invention is a method of administering a composition comprising piroxicam and ethylenediamine to a patient in need of analgesic treatment, wherein said composition is through an automated or gravity feed volume control devices, e.g., volutrol, minibag, large volume parenteral bag, and syringe driver.

With this embodiment, analgesic treatment can be administered to a patient in an efficient and rapid manner. Lower treatment costs can be obtained because there is no need to use prior dilution and/or administration through automated or gravity feed volume control devices, e.g., volutrol, minibag, large volume parenteral bag, syringe driver. Furthermore, alleviation of the pain can be accomplished more quickly because, for example, there is no need to dilute the pharmaceutical composition and administer it in a large volume over a longer period of time. Another advantage that can be obtained using certain NSAIDs in this embodiment is reduced administration errors by medical professionals. Because certain compositions of the invention can be administered by the intravenous route without the need for prior dilution and/or administration through automated or gravity feed volume control devices, e.g., volutrol, minibag, large volume parenteral bag, syringe driver, there is a lower risk of administration errors, e.g., incorrect dosage amount, incorrect infusion time, etc.

The dosage of the NSAID-piperazine or NSA/D-ethylenediamine pharmaceutical composition administered according to this embodiment will vary depending on a number of factors, including but not limited to type of subject (human or non-human), age, weight, medical history, and the like. Exemplary dosages include about 2.5 mg to about 500 mg per dose. For example, administration according to this embodiment of the present invention, wherein the composition comprises tenoxicam-ethylenediamine, tenoxicam-piperazine, piroxicam-ethylenediamine, ketoprofen-piperazine, or ketoprofen-ethylenediamine, may be given in about 5 to about 150 mg of active NSAID per adult dose.

In another embodiment, the present invention is directed to a method of administering an analgesic, anti-inflammatory, and/or antipyretic treatment into soft tissue at the site of, or in close proximity to the pain and inflammation, said method comprising administering a therapeutically effective amount of a pharmaceutical composition comprising a selected NSAID, preferably one or more NSA/Ds selected from the group consisting of ketoprofen, dexketoprofen, piroxicam, tenoxicam, and mixtures thereof, and a compound selected from piperazine, ethylenediamine and mixtures thereof; wherein said composition is administered directly into the soft tissue at or near the site of pain.

The pharmaceutical composition comprising the selected NSAID and the piperazine or ethylenediamine can be administered directly at the site of the pain or inflammation. Alternatively, the injection can be administered in close proximity to the site of pain, e.g., within 2 centimeters.

The dosage of the NSA/D-piperazine or NSAID-ethylenediamine pharmaceutical composition administered according to this embodiment will vary depending on a number of factors, including but not limited to type of subject (human or non-human), age, weight, medical history, and the like.

In certain aspects of this embodiment, the pharmaceutical composition comprising the selected NSAID, preferably one or more NSAIDs selected from the group consisting of ketoprofen, dexketoprofen, piroxicam, tenoxicam, and mixtures thereof, and a compound selected from piperazine and ethylenediamine and mixtures thereof, can be administered with significantly reduced discomfort, e.g., about 25, 50, 75, or 90% less discomfort, to the patient when compared to the administration of the NSAID alone administered over the same time period.

In another embodiment of the present invention, the method comprises administering a pharmaceutical composition comprising a selected NSAID, preferably one or more NSAIDs selected from the group consisting of ketoprofen, dexketoprofen, piroxicam, tenoxicam, and mixtures thereof, and a compound selected from piperazine and ethylenediamine, to a subject in need of analgesic, anti-inflammatory, and/or antipyretic treatment, wherein said composition is administered via topical application or infiltration of a dose to a surgical site or open wound for the treatment of acute or chronic pain, nociceptive and neuropathic pain, pre- and post-operative pain, cancer pain, pain associated with neurotransmitter dysregulation syndromes and orthopedic disorders.

The dosage of the NSAID-piperazine or NSAID-ethylenediamine pharmaceutical composition administered according to this embodiment will vary depending on a number of factors, including but not limited to type of subject (human or non-human), age, weight, medical history, surface area of the wound or surgical site, and the like. Exemplary dosages include about 0.01 mg to about 2000 mg per dose. For example, administration according to this embodiment of the present invention, wherein the composition comprises tenoxicam-piperazine, tenoxicam-ethylenediamine, piroxicam-ethylenediamine, ketoprofen-piperazine, or ketoprofen-ethylenediamine, may be given in about 1 to 1000 mg of active NSAID per adult dose.

In certain embodiments, the surgical site is a median sternotomy. In other embodiments, the site of administration is wound, such as a long bone fracture. The surgical or would site may be involve any site where localized attenuation of pain or inflammation is deemed desirable. Such surgical sites include but are not limited to a laparoscopy, a mastectomy, an arthroplasty, cancer surgery, bunionectomy, and the like. The surgical site may also be associated with an injury selected from the group consisting of a tear of the anterior cruciate ligament, a tear of the posterior cruciate ligament, a tear of the medial collateral ligament, a tear of the lateral collateral ligament; a meniscal cartilage tear; a cartilage defect of the knee; an orthopedic disorder of the shoulder selected from the group consisting of bursitis, dislocation, separation, impingement and tear of the rotator cuff, tendonitis, adhesive capsulitis, shoulder fracture, or may be associated with tendonitis, bursitis or bursitis injury, etc. Such surgery may be performed via laproscopy or otherwise. In one embodiment, the NSAID-piperazine or NSAID-ethylenediamine pharmaceutical composition may be administered in a pharmaceutically acceptable vehicle such as Sodium Chloride Injection, Ringers Injection, Isotonic Dextrose Injection, Sterile Water Injection, Dextrose, Lactated Ringers Injection and any combinations or mixtures thereof. In another embodiment, the NSAID-piperazine or NSA/D-ethylenediamine pharmaceutical composition may comprise an agent selected form the group consisting of antimicrobial agents, isotonic agents, buffers, antioxidants, local anesthetics, suspending and dispersing agents, emulsifying agents, sequestering, chelating agents and any combinations thereof. In another embodiment, the dose of the NSAID-piperazine or NSA/D-ethylenediamine pharmaceutical composition is administered in the form of microparticles selected from the group consisting of microcapsules and microspheres. In other embodiments, the selected NSAID-piperazine or selected NSAID-ethylenediamine pharmaceutical composition is administered in the form of sterile bandage, tape or patch or a sterile gel, cream or ointment

In another embodiment, the present invention is directed to a method of administering an analgesic, anti-inflammatory, and/or antipyretic treatment into an epidural, intrathecal, or intraarticular space, said method comprising administering to a subject a therapeutically effective amount of a pharmaceutical composition comprising a selected NSAID, preferably one or more NSAIDs selected from the group consisting of ketoprofen, dexketoprofen, piroxicam, tenoxicam, and mixtures thereof, and a base selected from piperazine and ethylenediamine, wherein said composition is administered directly into said epidural, intrathecal, or intraarticular space. Preferably, the NSA/D is selected from the group consisting of ketoprofen, dexketoprofen, and piroxicam.

Intrathecal doses of drugs are usually lower than intravenous doses of the same drug. In one embodiment, the intrathecal dose of the composition may be the same as its intravenous dose. In another embodiment, the intrathecal dose of the composition may be 25% of the usual intravenous dose. In a preferred embodiment, the intrathecal dose of the composition may be between 0.0001% and 20% of the usual intravenous dose. In a more preferred embodiment, the intrathecal dose of the composition may be between 0.01% and 10% of the usual intravenous dose.

Epidural doses of drugs are usually lower than intravenous doses of the same drug. In one embodiment, the epidural dose of the composition may be the same as its intravenous dose. In another embodiment, the epidural dose of the composition may be 80% of the usual intravenous dose. In a preferred embodiment, the epidural dose of the composition may be between 0.01% and 70% of the usual intravenous dose. In a more preferred embodiment, the epidural dose of the composition may be between 0.1% and 50% of the usual intravenous dose.

The subject of the method of the invention, in particular in each of the embodiments described above, is preferably a mammal and more preferably is a human. The subject can of course include other non-human animals, preferably horses, livestock, cattle, domesticated animals, cats, dogs, and the like.

In certain embodiments, the subject is a human. In particular, certain subpopulations of human patients are preferred subjects of certain embodiments. For example, in one embodiment, the composition of the invention is administered to a patient that is particularly prone to venous irritation, phlebitis, and/or injection pain. Such a patient may include an elderly patient, e.g., a patient 55 years of age or older. Alternatively, such a patient is at least 65, 75, or 85 years of age. Alternatively, a patient that has had multiple or repeated intravenous administrations may be particularly suited for treatment according to the present invention. Alternatively, a patient that has poor vascular access or is cachectic may be particularly suited for treatment according to the present invention.

In other embodiments, a method of the present invention can be used in dogs and cats for the short-term management of post-surgical pain. Alternatively, the method comprises administering to a dog or a cat a pharmaceutical composition as described herein for the long-term treatment of chronic pain, e.g., due to osteoarthritis. In preferred embodiments, the method comprises administering any of the specific compositions described herein comprising ketoprofen together with piperazine or ethylenediamine.

In another embodiment, a method of the present invention can be used in horses for the management of musculoskeletal pain due to soft tissue injury, synovitis, and osteoarthritis. In preferred embodiments, the method comprises administering any of the specific compositions described herein comprising ketoprofen together with piperazine, ethylenediamine, or mixtures thereof, to a horse as an IM injection. For example, the method of this embodiment comprises administering to a horse via IM injection a composition comprising ketoprofen; piperazine; and a pharmaceutically acceptable carrier, wherein said ketoprofen and piperazine are in a ratio of from about 1:4 to about 4:1, preferably about 1:2 to about 2:1. Alternatively, the method may comprise administering to a horse via IM injection a composition comprising ketoprofen; ethylenediamine; and a pharmaceutically acceptable carrier, wherein said ketoprofen and ethylenediamine are in a ratio of from about 1:2 to about 2:1.

In each of the above listed embodiments, the composition administered to the subject in need of the particular treatment or prevention contains the selected NSAID and the piperazine or ethylenediamine in a ratio of about 10:1 to about 1:10; in a ratio of about 5:1 to about 1:5; about 2:1 to about 1:2; in a ratio of about 1.1:1 to about 1:1.1; or in a ratio of about 1:1. Other embodiments include compositions wherein the ratio is about 10:1, 9:1, 8:1, 7:1, 6:1, 5:1, 4:1, 3:1, 2:1, 1:2, 1:3, 1:4, 1:5, 1:6, 1:7, 1:8, 1:9, and 1:10.

In another embodiment, the present invention is directed to a method of treating patent ductus arteriosus or intraventricular hemorrhage in an infant in need of such treatment, said method comprising administering to said infant a pharmaceutical composition comprising (a) one or more selected NSAIDs, preferably selected from the group consisting of ketoprofen, dexketoprofen, tenoxicam, piroxicam, and mixtures thereof; and (b) a compound selected from piperazine, ethylenediamine, and mixtures thereof.

Patent ductus arteriosus is a heart defect that occurs when the ductus arteriosus fails to close after birth. Normally, this duct closes. The ductus arteriosus is an open channel in the heart of a fetus which permits blood to bypass the lungs. The blood can bypass the lungs because the lungs are not used until after birth. Shortly after birth, the ductus arteriosus should close permanently under normal conditions. If this does not occur, patent ductus arteriosus is the result.

A patent ductus arteriosus can lead to the infant having difficulty breathing and poor growth. Other symptoms can include flooding of the lungs with blood and possibly acute heart failure.

In treating patent ductus arteriosus or intraventricular hemorrhage in an infant, particularly a premature infant, a pharmaceutical composition as described herein is administered. The administration of a pharmaceutical composition of the present invention to an infant afflicted with patent ductus arteriosus can help alleviate the symptoms and promote the closing of the patent ductus arteriosus.

In one embodiment of treating patent ductus arteriosus or intraventricular hemorrhage in an infant, a composition comprising ketoprofen and piperazine is administered to the infant. In another embodiment, a composition comprising ketoprofen and ethylenediamine is administered to the infant. In yet another embodiment, a composition of piroxicam and ethylenediamine is administered to the infant.

The methods and compositions described herein are useful for treating a wide range of specific types of pain. By way of a non-limiting example, in another embodiment, the present invention is used to prevent or treat postsurgical pain in hospitalized patients. In preferred embodiments, the method comprises administering any of the specific compositions described herein for the treatment of pain during or after orthopedic, neurologic, gynecologic, thoracic, urologic, or gastrointestinal surgery in hospitalized patients.

In another embodiment, a method of the present invention is used to prevent or treat postsurgical pain in hospitalized patients after major inpatient surgical procedures, e.g., total hip replacement, total knee replacement, hysterectomy, colectomy, prostatectomy, C-section, thoracotomy, laparotomy, laparoscopy, and the like.

In another embodiment, a method of the present invention is used to prevent or treat postsurgical pain in outpatient and day surgery patients after short duration surgical procedures, e.g., bunionectomy, arthroscopic surgery, skin and tissue biopsy, reduction and augmentation mammoplasty, mastectomy, rhinoplasty, rhytidectomy, abdominoplasty and the like. In another embodiment, a method of the present invention is used to prevent or treat nonsurgical acute pain in patients after burns, migraine, fracture, trauma, renal colic, low back pain, joint pain and the like. In another embodiment, a method of the present invention is used to prevent or treat chronic cancer and non-cancer pain, for example through the use of a continuous subcutaneous, intravenous, epidural or intrathecal implantable or external pump.

In an alternative embodiment, the compositions and methods disclosed herein are used to treat postsurgical pain from an appendectomy. The invention is used for treating this pain in adults as well as children. For example, in treating a child patient, the ketoprofen-piperazine, ketoprofen-ethylenediamine, or piroxicam-ethylenediamine composition can be administered in an amount of about 0.05 to about 15.0 mg/kg, preferably about 0.1 to about 2.0 mg/kg, based on the weight of the child patient.

When administering the compositions of the present invention to pediatric patients, the medical practitioner can vary the dose accordingly based on known dosing protocols for treating pediatric patients.

In another embodiment, a method of the present invention can be used to prevent or treat ocular itching and postsurgical pain, postsurgical inflammation and postsurgical photophobia after ophthalmic surgery.

The pharmaceutical composition can be administered in varying amounts and volumes. In a preferred embodiment, the ophthalmic volume of a single therapeutic dose does not exceed about 10 mL, 6 mL, 4 mL, 2 mL, 1 mL, 0.5 mL, 0.1 mL, 0.01 mL, or 0.001 mL. Thus, for example, the method of the invention may comprise administering, to a subject in need of analgesic or anti-inflammatory treatment, a pharmaceutical composition comprising piroxicam, ethylenediamine, and saline, with or without pH adjustment, additional excipients, buffers, surfactants, and the like

The dosage of the NSAID-piperazine or NSAID-ethylenediamine pharmaceutical composition administered according to this embodiment will vary depending on a number of factors, including but not limited to type of subject (human or non-human), age, weight, medical history, and the like. Exemplary ocular dosages include about 0.001 to 100 mg per dose. For example, administration according to the present invention, wherein the composition comprises piroxicam-ethylenediamine and ketoprofen-piperazine, or ketoprofen-ethylenediamine, may be given in about 0.1 mg and 5 mg of active NSAID per adult dose, respectively.

It is further understood that the various embodiments of the method of the present invention may utilize each of the various composition embodiments described herein.

### Common Parenteral Routes of Administration

Administration of the present invention can be via any parenteral route of administration. Non-limiting examples of suitable parenteral routes of administration are described in general terms as follows. Each of the following routes of administration can be used in the present invention, in particular in each of the specific embodiments described.

IM Administration. Once a popular method of drug administration, the intramuscular (IM) route is used considerably less frequently today due to improved availability of oral drugs and due to the ease of intravenous administration. This method of drug administration involves injecting drug directly into muscle mass, from where drug will gradually be absorbed systemically. For drugs that are not irritating when given by the IM route which require only a single injection, the IM route is still a viable route of administration, particularly in the outpatient setting. When repeated administration is required or a rapid and reliable onset is desired, the intravenous route is preferred as repeated IM administration can be painful and inconvenient. Commonly the deltoid, gluteus maximus and vastus lateralis are sites for IM injection.

Subcutaneous Administration involves injection into the subcutaneous fatty tissue under the skin. It can be used for the intermittent administration and self-administration of insulin and other medicines. In a small number of patients, particularly outpatients, this route is also used for the continuous or intermittent administration of drugs, usually with a programmable subcutaneous infusion device.

Epidural and Intrathecal Administration. Epidural and intrathecal infusions can provide effective analgesia, but require skilled personnel (usually an anesthetist) to put the systems in place. Catheters can be placed at any level of the spinal cord, although most commonly these techniques are used for pain in the lower part of the body. Epidural and intrathecal routes of administration are advantageous for difficult abdominal or pelvic pain. For short term use, epidural catheters can be placed percutaneously, and fixed either by secure taping or subcutaneous tunnelling. The drugs can then be delivered through a small pump or a syringe driver. Subjects can be ambulant and managed at home with these systems. However the primary care team must have the necessary training, knowledge and support. In patients with a longer prognosis, but who have a continuing source of pain, intrathecal systems, which can be fully implantable, have many advantages. These offer great freedom to the patient, as there is no external equipment and the pump only needs to be refilled every few weeks. Some of the pumps are programmable and offer great flexibility. Epidural and especially intrathecal doses of NSAIDs can be lower than intravenous doses. Epidural and intrathecal administration of NSAIDs can be used instead of intravenous administration to reduce systemic toxicity or to provide better efficacy. Epidural and intrathecal administration of NSAIDs can be also be used in conjunction with intravenous NSAIDs to provide additive or synergistic analgesia.

Intraarticular Administration. This method of administration can be used to provide analgesic and anti-inflammatory drugs directly into affected joints to relieve pain and inflammation, usually due to surgery, osteoarthritis or joint trauma.

Ocular administration. This method of administration can be used to provide analgesic and anti-inflammatory drugs directly into the eye to relieve pain, inflammation and photophobia after ophthalmic surgery.

Surgical site or open wound. This method of administration can be used when localized attenuation of pain or inflammation is deemed desirable and is achieved by topical, dermal administration or infiltration of a dose to a surgical site or open wound. The term “infiltration” shall mean administration into a discrete surgical site or open wound in a human or animal.

### Common Methods of Intravenous Administration

IV Drug Administration. The mode of N drug administration depends on the particular NSAID used, the patient's condition, and the desired clinical effects of the NSAID. The four primary modes of IV drug administration are continuous infusion, intermittent infusion, direct injection, and patient-controlled analgesia.

Continuous infusion typically refers to the admixture of a drug in a large volume of solution that is infused continuously over several hours to several days. The solution container is connected to an administration set, and the solubilized drug is infused through the venous access. The infusion may be administered by gravity feed or by use of electronic infusion control pump to deliver the drug accurately. Typically, continuous infusions can be used when the drug is highly diluted and constant plasma drug concentration should be maintained. Alternatively, continuous infusion can be used where large volumes of fluids and electrolytes need to be replenished along with the administration of the NSAID. Among the disadvantages with continuous infusion are possible fluid overload and potential incompatibilities between the infusion and other IV drugs administered through the same venous access device. Patient comfort may be compromised and patient mobility restricted.

Intermittent infusion refers to the administration of the drug as a small volume of fluid, typically 25 to 250 mL, and infused over, for example, 15 to 120 minutes at periodic intervals. Among the advantages of the intermittent method are the ability of the drug to produce higher peak blood concentrations at periodic intervals (compared with a continuous infusion), decreased risk of fluid overload, and greater convenience for the patient. Intermittent infusions may be given in a number of ways, including “piggyback” infusions through the established pathway of a primary infusion solution. Although the primary infusion is interrupted during the piggyback infusion, the drug from the intermittent infusion container mixes with the primary solution below the piggyback injection level. Thus, if this method is used, the drug and the primary solution must be compatible.

A second method to administer intermittent infusions is as a simultaneous infusion, where the drug is administered as a secondary infusion concurrently with the primary infusion. Instead of connecting the intermittent infusion at the piggyback port, it is attached to a lower secondary port. One potential disadvantage of this method is the tendency for blood to back up into the tubing once the secondary infusion has been completed, potentially occluding the venous access device. This generally does not occur with the piggyback method because the hydrostatic pressure closes the back check valve once the intermittent infusion is completed.

A third method is the use of a volume control set. Although it was originally designed to control the fluid volume delivered to the patient, a drug may be added to a small amount of solution in the volume control set and infused at the desired rate. It is still used in some pediatric settings because it limits the amount of fluid the child receives.

A fourth method for administering intermittent infusions is directly into the venous access device. The device is generally intended for intermittent administration, such as a peripheral heparin lock. The drug is added to a minibag or minibottle and infused intermittently. Between doses, the drug container and tubing are discarded.

Technological advances have produced alternatives for providing intermittent N doses, including drug powders that are attached between the primary solution and the infusion set. Once the drug vial is attached, the solution flows from the primary container through the drug vial and to the patient. Another innovation involves intermittent doses of drugs that are activated only at the time of use. Rather than preparing and refrigerating the drug before administration, the pharmacy simply dispenses the drug vial attached to a small container of solution. Immediately before administering the drug, the nurse activates the system by removing the barrier between the drug and the solution and agitating the container to achieve solubilization.

Direct injection, also known as IV push, TV bolus, or slow N injection, is the administration of a drug directly into the venous access device or through the proximal port of a continuous infusion set. A purpose is to achieve rapid plasma concentrations while avoiding costly and time consuming use of infusion devices. Instead of regulating drug administration by the infusion rate, a direct injection requires only the time it takes to push the syringe plunger. Since the drug may be incompatible with the infusing solution or heparin may be present in the intermittent device, the vascular access device can be flushed with normal saline before and after injecting the drug. Direct injections may require that the drug be drawn into a syringe before administration or that the drug be available in a prefilled syringe. A needle 1 inch or shorter should be used to administer the medication because longer needles may puncture the N tubing or the vascular access device. Another alternative is a needleless system, which also prevents inadvertent puncture of the tubing or device.

Patient-controlled analgesia (PCA) is the fourth method of administration of drug administration which promotes patient comfort through the self-administration of analgesic agents. With this method, an automated pump (PCA pump) is programmed to administer a small bolus of the drug when activated by the patient. The bolus amount (demand dose) and the time between doses (lock-out interval) are predetermined and programmed into the pump.

The treatment of pain inflammation and/or fever is frequently multimodal and involves the use of multiple drugs to provide optimal efficacy and safety. It is contemplated that the present invention may be used alone or in combination with other drugs to provide additive, complementary, or synergistic therapeutic effects, including other NSAIDs, COX-2 selective inhibitors, acetaminophen, propacetamol, tramadol, mu, delta and kappa receptor opioid agonists and antagonists, peripheral opioid receptor agonists, peripherally acting opioid receptor antagonists, local anesthetics, beta adrenergic agonists, alpha-2 agonists, selective prostanoid receptor antagonists; cannabinoid receptor agonists, NMDA receptor antagonists, neuronal nicotinic receptor agonists, calcium channel antagonists, sodium channel blockers, superoxide dismutase mimetics, p38 MAP kinase inhibitors, TRP1 (VR1) agonists, gabapentin, pregabalin, alpha(2)delta subunit of voltage-dependent Ca channels ligands, potassium channel ligands, antihistamines, and any other drugs that can be shown by a person proficient in the art to prevent or treat pain, inflammation, or fever.

Preferred combination therapies comprise the use of a composition of the present invention in combination with one or more compounds selected from the group consisting of acemetacin, ε-acetamidocaproic acid, acetaminophen, acetaminosalol, acetanilide, S-adenosylmethionine, alclofenac, alfentanil, allylprodine, alminoprofen, aloxiprin, alphaprodine, aluminum bis(acetylsalicylate), alvimopan, amfenac, amino chlorthenoxazin, 3-amino-4-hydroxybutyric acid, 2-amino-4-picoline, aminopropylori, aminopyrine, amixetrine, ammonium salicylate, anileridine, antrafenine, apazone, balsalazide, bendazac, benorylate, benoxaprofen, benzpiperylon, benzydamine, benzylmnorphine, berberine, bermoprofen, bezitramide, p-bromoacetanilide, bromosaligenin, bucetin, bucloxic acid, bucolome, bufexamac, bumadizon, bupivacaine, buprenorphine, butacetin, butibufen, butorphanol, carbamazepine, carbiphene, carsalam, chlorobutanol, chlorthenoxazin, choline salicylate, cinchophen, cinmetacin, ciramadol, clidanac, clometacin, clonitazene, clonixin, clopirac, codeine, cropropamide, crotethamide, desomorphine, dexoxadrol, dextromoramide, dezocine, diampromide, difenamizole, difenpiramide, dihydrocodeine, dihydrocodeinone dihydromorphine, dimenoxadol, dimepheptanol, dimethylthiambutene, dioxaphetyl butyrate, dipipanone, dipyrocetyl, dipyrone, ditazol, droxicam, emorfazone, enfenamnic acid, epirizole, eptazocine, etanercept, etersalate, ethenzamide, ethoheptazine, ethoxazene, ethylmethylthiambutene, ethylmorphine, etofenamate, etonitazene, eugenol, felbinac, fenclozic acid, fendosal, fentanyl, fentiazac, fepradinol, feprazone, floctafenine, flunoxaprofen, fluoresone, flupirtine, fluproquazone, fosfosal, gentisic acid, glafenine, glucametacin, guaiazulene, hydrocodone, hydromorphone, hydroxypethidine, ibufenac, ibuproxam, imidazole salicylate, indomethacin, indoprofen, infliximab, interleukin-10, isofezolac, isoladol, isomethadone, isonixin, isoxepac, ketobemidone, p-lactophenetide, lefetamine, levorphanol, lidocaine, lexipafant, lofentanil, lonazolac, meperidine, meptazinol, mesalamine, metazocine, methadone, methotrimeprazine, methylnaltrexone, metiazinic acid, metofoline, metopon, mofebutazone, mofezolac, morazone, morphine, morpholine salicylate, myrophine, nabumetone, nalbuphine, nalorphine, nefopam, nicomorphine, nifenazone, nimesulide, 5′-nitro-2′-propoxyacetanilide, norlevorphanol, normethadone, normorphine, norpipanone, olsalazine, opium, oxaceprol, oxametacine, oxaprozim, oxycodone, oxymorphone, papaveretum, paranyline, parsalmide, pentazocine, perisoxal, phenacetin, phenadoxone, phenazocine, phenazopyridine hydrochloride, phenocoll, phenoperidine, phenopyrazone, phenylbutazone, phenyl salicylate, phenyramidol, piketoprofen, piminodine, pipebuzone, piperylone, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalate, salverine, simetride, sodium salicylate, sufentanil, sulfasalazine, superoxide dismutase, suprofen, suxibuzone, tapentadol, tenoxicam, talniflumate, terofenamate, tetrandrine, thiazolinobutazone, tiaramide, tilidine, tinoridine, tolfenamic acid, tolmetin, tramadol, tropesin, viminol, xenbucin, ximoprofen, zaltoprofen, and ziconotide.

Particularly preferred combination therapies comprise the use of a composition of the present invention in combination with acetaminophen, alvimopan, morphine, meperidine, methylnaltrexone, hydromorphone, levorphanol, gabapentin, pregabalin, oxycodone, oxymorphone, tramadol, clonidine, ziconotide, methadone, nalorphine, nalbuphine, fentanyl, sufenatnil, alfentanil, remifentanil, lidocaine, mepivacaine, bupivacaine, levobupivacaine, dipyrone, pentazocine, tapentadol, ketbemidone, naloxone, naltrexone, or a derivative thereof.

The drug being used in combination therapy with the present invention can be administered by any route, including parenterally, orally, topically, transdermally, inhalationally, and the like.

### Compositions

Another aspect of the present invention is directed to a composition comprising: (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof.

The composition comprising the selected NSAID and piperazine or ethylenediamine may be in the form of a solid. In certain embodiments, the solid composition will be a crystalline form, e.g., a crystalline form of ketoprofen and piperazine or a crystalline form of ketoprofen and ethylenediamine. In other embodiments, the solid for may be amorphous, e.g., an amorphous form of ketoprofen and piperazine or an amorphous form of ketoprofen and ethylenediamine. Other embodiments may be a mixture of amorphous and crystalline forms, while still other compositions may be a glassy solid or a semi-solid.

The solid composition may be in the form of a salt, e.g., a salt between ketoprofen and piperazine or a salt between ketoprofen and ethylenediamine. In other embodiments, the solid composition may be a non-salt complex between the NSA/D and the piperazine, ethylenediamine, or mixtures thereof, having various ratios as described above.

In certain embodiments, the selected NSAID and the ethylenediamine and/or piperazine is a complex, preferably a salt. Exemplary complexes include a ketoprofen-piperazine complex, a ketoprofen-piperazine salt, a ketoprofen-ethylenediamine complex, a ketoprofen-ethylenediamine salt, a tenoxicam-ethylenediamine salt, a tenoxicam-piperazine-salt, a piroxicam-ethylenediamine salt, and a piroxicam-ethylenediamine complex.

In one embodiment, the composition comprises (a) an NSAID selected from the group consisting of ketoprofen, dexketoprofen, tenoxicam, and piroxicam;

(b) piperazine; and

(c) a pharmaceutically acceptable carrier.

In one embodiment, the composition comprises (a) an NSAID selected from the group consisting of ketoprofen, dexketoprofen, tenoxicam, and piroxicam;

(b) ethylenediamine; and

(c) a pharmaceutically acceptable carrier.

In another embodiment, the composition comprises (a) an NSAID selected from the group consisting of ketoprofen, dexketoprofen, tenoxicam, and piroxicam;

(b) piperazine; and

(c) ethylenediamine; and

(d) a pharmaceutically acceptable carrier.

In certain embodiments, the composition comprising the NSAID and piperazine contains the NSAID and the piperazine in a ratio of about 10:1 to about 1:10; about 5:1 to about 1:5; about 3:1 to about 1:3; about 2:1 to about 1:2; in a ratio of about 1.1:1 to about 1:1.1; or in a ratio of about 1:1. Other embodiments include compositions wherein the ratio is about 5:1, 4:1, 3:1, 2:1, 1:2, 1:3, 1:4, or 1:5.

In certain embodiments, the composition comprising the NSAID and ethylenediamine contains the NSAID and the ethylenediamine in a ratio of about 10:1 to about 1:10; about 5:1 to about 1:5; about 3:1 to about 1:3; about 2:1 to about 1:2; in a ratio of about 1.1:1 to about 1:1.1; or in a ratio of about 1:1. Other embodiments include compositions wherein the ratio is about 5:1, 4:1, 3:1, 2:1, 1:2, 1:3, 1:4, or 1:5.

In certain embodiments, the composition comprising the ketoprofen and piperazine contains ketoprofen and piperazine in a ratio of about 10:1 to about 1:10; about 5:1 to about 1:5; about 3:1 to about 1:3; about 2:1 to about 1:2; in a ratio of about 1.1:1 to about 1:1.1; or in a ratio of about 1:1. Other embodiments include compositions wherein the ratio is about 5:1, 4:1, 3:1, 2:1, 1:2, 1:3, 1:4, or 1:5.

In certain embodiments, the composition comprising ketoprofen and ethylenediamine contains ketoprofen and ethylenediamine in a ratio of about 10:1 to about 1:10; about 5:1 to about 1:5; about 3:1 to about 1:3; about 2:1 to about 1:2; in a ratio of about 1.1:1 to about 1:1.1; or in a ratio of about 1:1. Other embodiments include compositions wherein the ratio is about 5:1, 4:1, 3:1, 2:1, 1:2, 1:3, 1:4, or 1:5.

In certain embodiments, the composition comprising piroxicam and ethylenediamine contains piroxicam and ethylenediamine in a ratio of about 10:1 to about 1:10; about 5:1 to about 1:5; about 3:1 to about 1:3; about 2:1 to about 1:2; in a ratio of about 1.1:1 to about 1:1.1; or in a ratio of about 1:1. Other embodiments include compositions wherein the ratio is about 5:1, 4:1, 3:1, 2:1, 1:2, 1:3, 1:4, or 1:5.

The concentration of the selected NSAID, preferably selected from the group consisting of ketoprofen, dexketoprofen, tenoxicam, piroxicam, and mixtures thereof, in a parenteral formulation of the invention may vary as needed, e.g., from about 0.1 to about 200 mg/mL, preferably from about 1 to about 100 mg/mL, based on weight or potency of the NSAID. Other suitable values include about 1, 5, 10, 20, 25, 40, 50, 60, or 75 mg/mL.

The NSAID-piperazine or NSAID-ethylenediamine composition can be a parenteral drug formulation prepared, e.g., as a solid, liquid, semi-solid, or emulsion. The most common forms include solid, e.g., dry powder, crystalline, amorphous, lyophilized, and liquid formulations. Solid compositions can be reconstituted with a liquid vehicle just prior to administration. However, in many situations, it is particularly advantageous to provide a liquid formulation, more especially a ready-to-use or dilutable formulation. The formulation may optionally contain one or more additives, such as buffers, stabilizing agents, tonicity agents, antioxidant, anesthetics or bulking agent.

In another embodiment, the invention is directed to a stable parenteral formulation comprising a selected NSAID, preferably selected from the group consisting of ketoprofen, dexketoprofen, tenoxicam, piroxicam, and mixtures thereof; a compound selected from ethylenediamine and piperazine; and a pharmaceutically acceptable carrier. Such a composition can include, but is not limited to, dry powders, lyophilized preparations, and ready to use solutions.

For example, in one embodiment of the invention, a liquid pharmaceutical composition comprises a selected NSAID, as described above, preferably selected from the group consisting of ketoprofen, dexketoprofen, piroxicam, tenoxicam, and mixtures thereof; a compound selected from the group consisting of piperazine and ethylenediamine; and a liquid carrier. Liquid carriers and excipients are known in the art. See, e.g., Remington's. Suitable liquid carriers and excipients include, but are not limited to, water, saline, ethanol, benzyl alcohol, etc., and mixtures thereof.

In one embodiment, a preferred carrier is water, in particular water for injection (WFI).

The pharmaceutical composition may contain a buffer. The term “buffer” refers to a pharmaceutically acceptable excipient that helps to maintain the pH of the solution within a particular range specific to the buffering system. The buffer is present for example at a concentration in the range from about 0.03% to about 5.0% w/v, or about 0.1% to about 2.0% w/v. Non-limiting illustrative examples of pharmaceutically acceptable buffering agents include phosphates, 2-amino-2-(hydroxymethyl)-1,3-propanediol (“tris”), ascorbate, acetates, citrates, tartrates, lactates, succinates, amino acids and maleates and the like. Buffers are known in the art. See, e.g., Remington's.

The pH of a liquid formulation of the present invention in preferred embodiments is generally from about 5 to about 9, preferably from about 6 to about 8. In other embodiments, the pH of the liquid formulation is about 5, 6, 7, 8, or 9. Alternatively, the pH of the liquid formulation is about 7.5. Alternatively, the pH of the liquid formulation may be selected from the following ranges: 6.0 to 6.9; 6.5 to 6.9; 7 to 7.5; 7.6 to 8.0; 7.6 to 8.5; and 7.6 to 9.0.

The concentration and dosage of the NSAID in the liquid parenteral formulation can vary as needed. For example, the NSAID-piperazine or NSA/D-ethylenediamine complex can be in an amount of about 1 mg/mL to about 200 mg/mL, alternatively from about 2 mg/mL to about 200 mg/mL, based on the mass of the NSAID. Furthermore, the liquid formulation may be packaged in any suitable container, for example, a vial, ampule, bag, bottle, prefilled syringe, and the like. Preferably, the liquid formulation comprises a physiologically compatible fluid, such as sterile, buffered saline.

The liquid carrier used in preferred embodiments is preferably injection-quality water, by itself or preferably with the addition of conventional, physiologically tolerated solvents and/or solubilizing agents, e.g., propylene glycol, polyols such as glycerol, polyoxyalkylenes, e.g., poly(oxyethylene)-poly(oxypropylene) polymers, glycerol-formal, benzyl alcohol, or butanediol. The addition of solubilizing agents produces a composition which is stable at low temperatures and minimizes or prevents partial crystallization of the selected NSAID, in spite of the high concentration NSAID that may be present.

In each of the embodiments described herein, the liquid pharmaceutical formulation may be characterized in terms of its osmolarity. In certain embodiments, the osmolarity of the pharmaceutical formulation is from about 50 to about 1000 mOsm/L, preferably about 100 to about 500 mOsm/L. In other embodiments, the pharmaceutical compositions of the present invention are prepared such that the osmolarity is about 200 to about 300 mOsm/L, about 250 to about 350 mOsm/L, about 270 to about 330 mOsm/L, about 270 to about 290 mOsm/L, or about 280 to about 300 mOsm/L, or is 270, 280, 290, 300, or 310 mOsm/L. Alternative preferred embodiments include compositions which have a lower osmolarity than physiological osmolarity.

For therapeutic use, the injection preparations according to the invention can be sterilized by conventional methods or filled under sterile conditions into ampules.

The concentration of the selected NSA/D in the liquid pharmaceutical composition can vary and may depend on the intended use. In certain embodiments, the injection solutions according to the invention contain from about 5 to about 50% ketoprofen.

The pharmaceutical composition may contain one or more solubilizing agents. Thus, in one embodiment, the invention encompasses a liquid pharmaceutical composition, in particular, for parenteral administration, comprising ketoprofen and piperazine in a ratio of 2:1 in a solvent, the solvent consisting of 10-70 weight percent, preferably 20-50 weight percent, of a mixture of (a) propylene glycol and (b) polyethylene glycol and 90-30 weight percent, preferably 80-50 weight percent, of water, and in the solvent mixture the weight ratio of propylene glycol:polyethylene glycol being between about 9.5:0.5 and about 0.5:9.5, preferably between about 3:1 and about 1:3.

Thus, in one embodiment, the invention encompasses a liquid pharmaceutical composition, in particular, for parenteral administration, comprising (a) ketoprofen or piroxicam and (b) ethylenediamine, in a ratio of 2:1 in a liquid carrier, the liquid carrier comprising of 10-70 weight percent, preferably 20-50 weight percent, of a mixture of (a) propylene glycol and (b) polyethylene glycol and 90-30 weight percent, preferably 80-50 weight percent, of water, and in the solvent mixture the weight ratio of propylene glycol:polyethylene glycol being between about 9.5:0.5 and about 0.5:9.5, preferably between about 3:1 and about 1:3.

According to the invention, liquid formulations of invention may employ one or more stabilizing agents. A stabilizing agent may slow, delay, reduce, or prevent the precipitation of an NSA/D in free acid form. It will be understood that the effectiveness of such means for stabilizing the NSAID salt, illustrative examples of which are individually described in further detail below, depend on, inter alia, composition of the particular solvent liquid, selection and amount of NSA/D salt, and desired final presentation of the composition.

One class of suitable salt stabilizing means, particularly for a PEG-containing composition of the invention, is a means for limiting effective exposure of the composition to oxygen. The term “limiting effective exposure of the composition to oxygen” includes placing the composition in contact with an oxygen-limited microatmosphere and/or including in the composition one or more excipients or agents that mitigate potential deleterious effects of oxygen. Limiting the effective exposure of the composition to oxygen can be accomplished by one or more of the illustrative, nonlimiting means described more fully immediately below.

One means for limiting effective exposure of the composition to oxygen is to place the composition in contact with an oxygen-limited microatmosphere in a sealed container. Such a container can have a substantial internal headspace occupied by a microatmosphere having low oxygen pressure. Alternatively, the container can have very little or no headspace, in which case effective exposure of the composition to oxygen is limited largely by the barrier effect provided by the sealed container itself. Any suitable pharmaceutical container can be used to prepare an article of manufacture according to this embodiment. The container can be a multi-dose container, enclosing an amount of the composition preferably corresponding to 2 to about 30, for example about 4 to about 20, unit doses. Alternatively, the container encloses an amount of the composition corresponding to a single unit dose. Such a single-dose article of manufacture has the further advantage of eliminating a measuring step before administration of the composition. Since compositions of the invention are desirable for parenteral administration, the container preferably is sufficient to maintain sterility of a composition contained therein. The container can also be used to facilitate direct administration (without need for transfer to another vessel or container) of a composition of the invention, e.g., a syringe. Non-limiting examples of suitable containers for an article of manufacture of this invention include vials of any shape and/or size, ampoules, syringes, packets, pouches, auto-injectors, etc. In one embodiment, the container further comprises means to protect the composition from exposure to light, e.g., amber glass walls.

A composition of the invention can be sealed in a container in any suitable manner including but not limited to frictionally- and/or hermetically-induced seals. Such a seal can illustratively be provided by a stopper made of rubber or other polymeric material. A preferred seal comprises an inert coating, for example a coating of a fluoropolymer such as polytetrafluoroethylene, e.g., Teflon®, to prevent chemical interaction between the composition and the seal. The seal can illustratively be secured by a metal over-cap and/or an external cover, e.g., plastic, until use. Optionally, the seal can comprise at least one septum or thinner area of sealing material through which a needle can be inserted to extract the composition without cracking or breaking any glass or plastic portion of container wall. Regardless of what form of seal is used, such a seal should substantially inhibit movement of gas into or out of the container until the seal is penetrated for use of the composition present in the container.

Even where the composition is enclosed in a sealed container with an oxygen limited micro atmosphere, effective exposure of the composition to oxygen can be further limited by one or more of the following means: 1) a container size and/or shape that substantially maximizes fill volume and/or substantially minimizes headspace volume; 2) low oxygen pressure in the headspace; 3) use in the solvent liquid of water which has been purged of molecular oxygen; and 4) use of a grade of PEG having a low peroxide content, for example not greater than about 1.5 meq/kg and preferably not greater than about 1.0 meq/kg.

The term “headspace” or “headspace volume” with respect to an article of manufacture of the invention refers to any interior volume of the container that is not occupied by, but is in contact with, the composition. Generally, the headspace volume is occupied by a gaseous medium. The term “fill volume” with respect to an article of manufacture of the invention refers to any interior volume of the container that is occupied by the composition.

The term “total volume” refers to the entire interior volume of the container and may also be referred to elsewhere as overflow volume; total volume generally equals the sum of the fill and headspace volumes.

Yet another suitable means for limiting effective exposure of a composition, particularly a PEG-containing composition, of the invention to oxygen, and thereby providing said NSAID salt stabilizing means, comprises one or more pharmaceutically acceptable antioxidants, preferably free-radical scavenging antioxidants, as a component of the solvent liquid. Non-limiting illustrative examples of suitable antioxidants include α-tocopherol (vitamin E), ascorbic acid (vitamin C) and salts thereof including sodium ascorbate and ascorbic acid palmitate, butylated hydroxyanisole (BHA), butylated hydroxytoluene (BHT), cystein, cysteinate HCl, dithionite sodium, ethylenediamine tetraacetic acid, fumaric acid, gentisic acid and salts thereof, hypophosphorous acid, malic acid, methionine, monothioglycerol, N-acetyl-cysteine, alkyl gallates, for example propyl gallate, octyl gallate and lauryl gallate, sodium sulfite, sodium bisulfite, sodium and potassium metabisulfite, thioglycolate sodium, ethanolamine, glutamate monosodium, formaldehyde, sulfoxylate sodium and monothioglycerol. Preferred free radical-scavenging antioxidants are alkyl gallates, vitamin E, BHA, BHT, ascorbate and methionine, more especially BHA, ascorbate and methionine. Preferably, an antioxidant is selected that is substantially soluble in the particular solvent liquid employed and does not result in changes to the composition which are detectable by unaided sensory organs (e.g., color or odor changes). BHA is an illustrative preferred antioxidant for use in a composition of the invention. If included, one or more antioxidants are preferably present in a composition of the invention in a total antioxidant amount of about 0.001% to about 5%, preferably about 0.001% to about 2.5%, and still more preferably about 0.001% to about 1%, by weight.

When freeze dried, the formulations may optionally contain a bulking agent. The term “bulking agent” refers to a pharmaceutically acceptable excipient that adds bulk to a formulation which results in a well-formed cake upon freeze drying. The bulking agent is for example present in a formulation at a concentration in the range from about 1% to about 60% w/v, or about 3% to about 50% w/v. Nonlimiting examples of suitable bulking agents include mannitol, glycine, lactose, sucrose, trehalose, dextran, hydroxyethyl starch, ficoll and gelatin.

The solid composition also may include a tonicity agent. Suitable tonicity agents include, but are not limited to, glycerin, lactose, mannitol, dextrose, sodium chloride, sodium sulfate, and sorbitol.

Non-limiting examples of suitable nonaqueous solubilizers that can be present in the solvent liquid include polyethylene glycol (PEG), ethanol, dimethylacetamide (DMAC), propylene glycol, and mixtures thereof. It is preferred that the solvent liquid comprise at least one of PEG, DMAC, and ethanol.

Yet another means for stabilizing the formulation in a PEG-containing composition is a metal sequestering agent or chelating agent. Non-limiting examples of suitable sequestering agents include ethylenediamine tetraacetic acid (EDTA), potassium polyphosphate, sodium polyphosphate, potassium metaphosphate, sodium metaphosphate, dimethylglyoxime, 8-hydroxyquinoline, nitrilotriacetic acid, dihydroxyethylglycine, gluconic acid, citric acid and tartaric acid.

The composition of the present invention can optionally contain a surfactant. Non-limiting examples of suitable surfactants include cetrimide, docusate sodium, glyceryl monooleate, sodium lauryl sulfate, or sorbitan esters. The surfactant may optionally be a polyoxyethylenesorbitan fatty acid ester. Polyoxyethylenesorbitan fatty acid esters are also referred to as polysorbates, e.g., polysorbate 80 (polyoxyethylene sorbitan monooleate, Tween 80), polysorbate 40 and polysorbate 20.

The composition of the present invention can optionally be manufactured in glass-lined or a “greater than or equal to 316 temper-grade” steel tank.

Oxygen pressure in a container headspace of an article of manufacture of the invention can be limited in any suitable manner, illustratively by placing nitrogen and/or a noble gas (collectively referred to herein as “inert gases”) in the container headspace. In this embodiment, the headspace volume preferably comprises one or more inert gases selected from the group consisting of nitrogen, helium, neon and argon. One way to ensure low oxygen pressure in the headspace is to prepare, fill and seal the container under an atmosphere of inert gas and/or to flush the container headspace with inert gas after filling, illustratively using parallel in-line flushing. An inert gas atmosphere can illustratively be provided using a zero oxygen tunnel commercially available from Modified Atmosphere Packaging Systems of Des Plaines, Ill., or by using a nitrogen or noble gas atmosphere glove bag.

Various polymorphs of this invention may be prepared by crystallization under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or slow cooling. The presence of polymorphs may be determined by solid probe NMR spectroscopy, IR spectroscopy, differential scanning calorimetry, powder X-ray data or such other techniques.

Pharmaceutically acceptable solvates of the invention may be prepared by conventional methods such as dissolving the compounds in solvents such as water, methanol, ethanol etc., and recrystallizing by using different crystallization techniques.

In an additional embodiment, the composition of the present invention comprises: (a) one or more selected NSAIDs, in racemic, enantiomeric excess, or enantiomeric form, preferably selected from the group consisting of ketoprofen, dexketoprofen, piroxicam, tenoxicam, and mixtures thereof; (b) a compound selected from ethylenediamine, piperazine, and mixtures thereof; (c) sterile water; (d) optionally one or more pharmaceutically acceptable buffers; and (e) optionally one or more pharmaceutically acceptable preservatives; and wherein the pH is from about 6.5 to about 8.5, preferably from about 7.0 to about 8.0; the ratio of said NSAID to said compound is about 3:1 to about 1:3, preferably from about 2:1 to about 1:2; and the concentration of said NSAID is about 0.1 mg/mL to about 100 mg/mL, preferably about 1, 5, 10, 20, 25, 40, 50, 60, or 75 mg/mL. In a preferred embodiment, the composition is contained in a sealed glass vial.

For example, the composition of the present invention comprises: (a) ketoprofen; (b) ethylenediamine; (c) sterile water; (d) optionally one or more pharmaceutically acceptable buffers; and (e) optionally one or more pharmaceutically acceptable preservatives; and wherein the pH is from about 6.5 to about 8.5, preferably from about 7.0 to about 8.0; the ratio of said ketoprofen to said ethylenediamine is about 3:1 to about 1:3, preferably about 2:1; and the concentration of said ketoprofen is about 1 mg/mL to about 100 mg/mL, preferably about 25, 40, 50, 60, or 75 mg/mL. In a preferred embodiment, the composition is contained in a sealed glass vial.

Alternatively, the composition of the present invention comprises: (a) ketoprofen; (b) piperazine; (c) sterile water; (d) optionally one or more pharmaceutically acceptable buffers; and (e) optionally one or more pharmaceutically acceptable preservatives; and wherein the pH is from about 6.5 to about 8.5, preferably from about 7.0 to about 8.0; the ratio of said ketoprofen to said piperazine is about 3:1 to about 1:3, preferably about 1:1; and the concentration of said ketoprofen is about 1 mg/mL to about 100 mg/mL, preferably about 25, 40, 50, 60, or 75 mg/mL. In a preferred embodiment, the composition is contained in a sealed glass vial.

Alternatively, the composition of the present invention comprises: (a) piroxicam; (b) ethylenediamine; (c) sterile water; (d) one or more pharmaceutically acceptable buffers; and (e) optionally one or more pharmaceutically acceptable preservatives; and wherein the pH is from about 6.5 to about 8.5, preferably from about 7.0 to about 8.0; the ratio of said piroxicam to said ethylenediamine is about 3:1 to about 1:3, preferably about 2:1; and the concentration of said piroxicam is about 0.1 mg/mL to about 50 mg/mL, preferably about 1, 5, 10, or 20 mg/mL. In a preferred embodiment, the composition is contained in a sealed glass vial.

Alternatively, the composition of the present invention comprises: (a) tenoxicam; (b) ethylenediamine; (c) sterile water; (d) one or more pharmaceutically acceptable buffers; and (e) optionally one or more pharmaceutically acceptable preservatives; and wherein the pH is from about 6.5 to about 8.5, preferably from about 7.0 to about 8.0; the ratio of said piroxicam to said ethylenediamine is about 3:1 to about 1:3, preferably about 2:1; and the concentration of said piroxicam is about 0.1 mg/mL to about 50 mg/mL, preferably about 1, 5, 10, or 20 mg/mL. In a preferred embodiment, the composition is contained in a sealed glass vial.

In an additional embodiment, the present invention comprises a sealed container, e.g., a glass vial, containing a composition comprising: (a) one or more selected NSAIDs, in racemic, enantiomeric excess, or enantiomeric form, preferably selected from the group consisting of ketoprofen, dexketoprofen, piroxicam, tenoxicam, and mixtures thereof; (b) a compound selected from ethylenediamine, piperazine, and mixtures thereof; (c) sterile water; (d) optionally one or more pharmaceutically acceptable buffers; and (e) optionally one or more pharmaceutically acceptable preservatives; and wherein the pH is from about 6.5 to about 8.5, preferably from about 7.0 to about 8.0; the ratio of said NSAID to said compound is about 3:1 to about 1:3, preferably from about 2:1 to about 1:2; and the concentration of said NSAID is about 0.1 mg/mL to about 100 mg/mL, preferably about 1, 5, 10, 20, 25, 40, 50, 60, or 75 mg/mL; and wherein the composition shows no substantial visible signs of crystallization and/or significant deviation, e.g., less than or equal to 5% of original value, in pH and/or osmolarity, and/or contains 100%±5% original NSAID content.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein the composition is substantially free of a surfactant. Other aspects of this embodiment include any of the above described embodiments of the pharmaceutical composition wherein the composition is substantially free of a surfactant. For example, a further embodiment of the invention is a pharmaceutical composition comprising ketoprofen, ethylenediamine, and a pharmaceutically acceptable carrier; wherein the composition is substantially free of a surfactant.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of a benzyl alcohol. Other aspects of this embodiment include any of the above described embodiments of the pharmaceutical composition wherein the composition is substantially free of a benzyl alcohol. For example, a further embodiment of the invention is a pharmaceutical composition comprising ketoprofen, ethylenediamine, and a pharmaceutically acceptable carrier; wherein the composition is substantially free of a benzyl alcohol.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of a polyethylene glycol (PEG). Other aspects of this embodiment include any of the above described embodiments of the pharmaceutical composition wherein the composition is substantially free of a polyethylene glycol (PEG). For example, a further embodiment of the invention is a pharmaceutical composition comprising ketoprofen, ethylenediamine, and a pharmaceutically acceptable carrier; wherein the composition is substantially free of a polyethylene glycol (PEG).

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of a N-methylglucamine. Other aspects of this embodiment include any of the above described embodiments of the pharmaceutical composition wherein the composition is substantially free of a N-methylglucamine. For example, a further embodiment of the invention is a pharmaceutical composition comprising ketoprofen, ethylenediamine, and a pharmaceutically acceptable carrier; wherein the composition is substantially free of a N-methylglucamine.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of arginine or derivatives thereof. Other aspects of this embodiment include any of the above described embodiments of the pharmaceutical composition wherein the composition is substantially free of arginine or derivatives thereof. For example, a further embodiment of the invention is a pharmaceutical composition comprising ketoprofen, ethylenediamine, and a pharmaceutically acceptable carrier; wherein the composition is substantially free of arginine or derivatives thereof.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of lysine or derivatives thereof. Other aspects of this embodiment include any of the above described embodiments of the pharmaceutical composition wherein the composition is substantially free of lysine or derivatives thereof. For example, a further embodiment of the invention is a pharmaceutical composition comprising ketoprofen, ethylenediamine, and a pharmaceutically acceptable carrier; wherein the composition is substantially free of lysine or derivatives thereof.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of one or more of, or all of, alkylammonium salts, i.e., tromethamine, dropopizine, 3-(4-phenyl-1-piperazinyl)-1,2-propanediols, and derivatives thereof. Other aspects of this embodiment include any of the above described embodiments of the pharmaceutical composition wherein the composition is substantially free of one or more of, or all of, alkylammonium salts, such as tromethamine, dropopizine, and derivatives thereof. For example, a further embodiment of the invention is a pharmaceutical composition comprising ketoprofen, ethylenediamine, and a pharmaceutically acceptable carrier; wherein the composition is substantially free of one or more of, or all of, alkylammonium salts, i.e., tromethamine, dropopizine, 3-(4-phenyl-1-piperazinyl)-1,2-propanediols, and derivatives thereof.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of an adjuvant. Other aspects of this embodiment include any of the above described embodiments of the pharmaceutical composition wherein the composition is substantially free of an adjuvant. For example, a further embodiment of the invention is a pharmaceutical composition comprising ketoprofen, ethylenediamine, and a pharmaceutically acceptable carrier; wherein the composition is substantially free of an adjuvant.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of citric acid or derivatives thereof. Other aspects of this embodiment include any of the above described embodiments of the pharmaceutical composition wherein the composition is substantially free of citric acid or derivatives thereof. For example, a further embodiment of the invention is a pharmaceutical composition comprising ketoprofen, ethylenediamine, and a pharmaceutically acceptable carrier; wherein the composition is substantially free of citric acid or derivatives thereof.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of glycine or derivatives thereof. Other aspects of this embodiment include any of the above described embodiments of the pharmaceutical composition wherein the composition is substantially free of glycine or derivatives thereof. For example, a further embodiment of the invention is a pharmaceutical composition comprising ketoprofen, ethylenediamine, and a pharmaceutically acceptable carrier; wherein the composition is substantially free of glycine or derivatives thereof.

In other embodiments, the composition of the present invention is directed to a pharmaceutical composition comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof; wherein said composition is substantially free of glycerol or derivatives thereof. Other aspects of this embodiment include any of the above described embodiments of the pharmaceutical composition wherein the composition is substantially free of glycerol or derivatives thereof. For example, a further embodiment of the invention is a pharmaceutical composition comprising ketoprofen, ethylenediamine, and a pharmaceutically acceptable carrier; wherein the composition is substantially free of glycerol or derivatives thereof.

In certain embodiments of the composition comprising a NSAID-ethylenediamine or NSA/D-piperazine complex, the selected NSAID may be in the form of a solvate, polymorph, hydrate, conjugate, ester or prodrug in racemic, enantiomeric excess or enantiomeric form, or mixture thereof.

In another embodiment, the present invention is directed to a composition comprising a NSA/D-ethylenediamine or NSA/D-piperazine complex, preferably crystalline, having an aqueous solubility of greater than about 300 mg/mL, about 350 mg/mL, about 400 mg/mL, about 450 mg/mL, or about 500 mg/mL. In preferred embodiments, the composition comprises a ketoprofen-piperazine complex having an aqueous solubility of greater than about 360 mg/mL. Another embodiment comprises a ketoprofen-ethylenediamine complex having an aqueous solubility of greater than about 500 mg/mL.

In another instance, the composition of the invention comprises a ketoprofen-piperazine complex having an aqueous solubility of greater than about 360 mg/mL and wherein the ratio of said the ketoprofen to the piperazine is about 1 to 2.

In another instance, the composition of the invention comprises a ketoprofen-ethylenediamine complex having an aqueous solubility of greater than about 500 mg/mL and wherein the ratio of said the ketoprofen to the piperazine is about 1 to 2.

Additionally, the present invention is directed to sealed syringe comprising a sterile solution comprising (a) one or more selected NSAIDs, in racemic, enantiomeric excess, or enantiomeric form, preferably selected from the group consisting of ketoprofen, dexketoprofen, piroxicam, and mixtures thereof; and (b) a compound selected from ethylenediamine, piperazine, tenoxicam, and mixtures thereof. For example, a solution comprising water, e.g., purified by reverse osmosis, piperazine, and ketoprofen, and optionally sorbitol, and adjusted to a pH from about 6.5 to about 8.5 is added to presterilized syringes under nitrogen atmosphere and aseptic conditions. The solution is optionally first filtered through a filter, e.g., a 0.45 micron filter, and deoxygenated with nitrogen or deoxygenated and finally passed through 0.22 micron membrane filter into presterilized syringes under nitrogen atmosphere and aseptic conditions. The syringes are sealed under an inert atmosphere, e.g., nitrogen. The sealed syringe containing the NSAID-piperazine or NSAID-ethylenediamine complex can be used in accordance with the methods described herein. In preferred embodiments, the solutions in the sealed syringe show no visible signs of crystallization or significant deviation in pH and osmolarity and contain 100%±5% original NSA/D content when stored for 12 weeks at 5° C., 25° C./60% RH, 30° C./60% RH, 40° C./75% RH as determined by HPLC.

The osmolality of the solution in the sealed syringe may be from about 200 to about 400, preferably from about 280 to about 300 mOsm/L.

Additionally, the present invention is directed to an ampoule comprising a sterile solution comprising (a) one or more selected NSAIDs, in racemic, enantiomeric excess, or enantiomeric form, preferably selected from the group consisting of ketoprofen, dexketoprofen, piroxicam, tenoxicam, and mixtures thereof; and (b) a compound selected from ethylenediamine, piperazine, and mixtures thereof. For example, a solution comprising water, e.g., purified by reverse osmosis, piperazine, and ketoprofen, and optionally sorbitol, and adjusted to a pH from about 6.5 to about 8.5 is added to presterilized vial under nitrogen atmosphere and aseptic conditions. The solution is optionally first filtered through a filter, e.g., a 0.45 micron filter, and deoxygenated with nitrogen or deoxygenated and finally passed through 0.22 micron membrane filter into a presterilized vial under nitrogen atmosphere and aseptic conditions. The vial is sealed under an inert atmosphere, e.g., nitrogen. The sealed vial, or ampoule, containing the NSAID-piperazine or NSAID-ethylenediamine complex can be used in accordance with the methods described herein. In preferred embodiments, the solution in the ampoule shows no visible signs of crystallization or significant deviation in pH and osmolarity and contain 100%±5% original NSAID content when stored for 12 weeks at 5° C., 25° C./60% RH, 30° C./60% RH, 40° C./75% RH as determined by HPLC.

The osmolality of the solution at in the ampoule may be from about 200 to about 400, preferably from about 280 to about 300 mOsm/L.

Furthermore, it is understood that each of the embodiments disclosed herein may alternatively be described as “consisting of” or “consisting essentially of” the listed components. For example, one embodiment disclosed above is directed to a composition comprising (a) an NSAID selected from the group consisting of ketoprofen, dexketoprofen, tenoxicam, and piroxicam; (b) piperazine; and (c) a pharmaceutically acceptable carrier. It is understood that the composition may also consist, or consist essentially, of (a) an NSAID selected from the group consisting of ketoprofen, dexketoprofen, and piroxicam; (b) piperazine; and (c) a pharmaceutically acceptable carrier.

Additionally, it is understood that each of the various embodiments of the pharmaceutical compositions described herein may be used with each of the various embodiments of the described method of the present invention as described herein.

### Preparing the Compositions

In certain embodiments, the compositions of the present invention can be prepared by direct salification between an appropriate NSAID acid, preferably selected from the group consisting of ketoprofen, dexketoprofen, tenoxicam, piroxicam, and mixtures thereof, and ethylenediamine, piperazine, and mixtures thereof.

The acid addition salts may be prepared by salifying the amino function of the ethylenediamine or piperazine with the carboxylic acid function of the selected NSAID in a stoichiometric or nonstoichiometric amount, preferably in a stoichiometric amount, depending on the objectives. Advantageously, the reaction medium may be an organic solvent in which the selected NSAID and ethylenediamine or piperazine are mutually soluble, such as ethyl alcohol, wherein the selected NSAID is first dissolved and then the ethylenediamine or piperazine, with mixing, to form a clear solution. The clear solution can be concentrated, as by evaporation of the organic solvent, or the salt can be separated from the reaction medium such as by precipitation or crystallization, all as described in the aforementioned and known to those of skill in the art.

The direct salification is often carried out in a medium which is predominantly aqueous, at moderate temperatures around ambient temperature, and for periods in the order of 1 or 6 hours. The acid, which is insoluble in an aqueous medium, is added to a solution or partial suspension of ethylenediamine or piperazine in a stoichiometric or nonstoichiometric amount, preferably in a stoichiometric amount, depending on the objectives, and gradually goes into solution as the salifcation proceeds. Finally, the product can be isolated by, for example, lyophilization or precipitation with appropriate solvents. More often, the preparation is carried out in the presence of an excess of an organic solvent, for example of a C₁₋₄ alcohol or acetone, and, in this case, the salt precipitates or crystallizes directly from the aqueous-alcoholic or aqueous-acetone medium. In general, high yields are obtained.

For example, in one embodiment, a composition comprising an NSA/D and piperazine is prepared by adding the NSAID and the piperazine in appropriate amounts to a volume of water, optionally with sonication to facilitate dissolution; and then obtaining the NSAID piperazine composition after the water has evaporated.

The concentration of the NSAID in the solution prepared as described can vary. In certain embodiments, the NSAID concentration is from about 0.01 M to about 10 M, from about 0.05 M to about 5 M, or from about 0.1 M to about 3 M. In other embodiments, the NSAID concentration is about 0.1 M, 0.3 M, 0.5 M, 0.7 M, or 0.9 M.

The concentration of the piperazine in the solution prepared as described can vary. In certain embodiments, the piperazine concentration is from about 0.01 M to about 20 M, from about 0.05 M to about 10 M, or from about 0.1 M to about 8 M. In other embodiments, the piperazine concentration is about 0.5 M, 0.7 M, 0.9 M, 1 M, 1.3 M, or 1.5 M.

For example, in another embodiment, a composition comprising an NSAID, preferably one or more NSAIDs selected from the group consisting of ketoprofen, dexketoprofen, piroxicam, and mixtures thereof, and ethylenediamine is prepared by adding the NSAID and the ethylenediamine in appropriate amounts to a volume of water, optionally with sonication to facilitate dissolution; and then obtaining the NSAID ethylenediamine composition after the water has evaporated.

The concentration of the NSA/D in the solution prepared as described can vary. In certain embodiments, the NSAID concentration is from about 0.01 M to about 10 M, from about 0.05 M to about 5 M, or from about 0.1 M to about 3 M. In other embodiments, the NSAID concentration is about 0.1 M, 0.3 M, 0.5 M, 0.7 M, or 0.9 M.

The concentration of the ethylenediamine in the solution prepared as described can vary. In certain embodiments, the ethylenediamine concentration is from about 0.01 M to about 20 M, from about 0.05 M to about 10 M, or from about 0.1 M to about 8 M. In other embodiments, the ethylenediamine concentration is about 0.5 M, 0.7 M, 0.9 M, 1 M, 1.3 M, or 1.5 M.

The solution of the selected NSAID, preferably one or more NSAIDs selected from the group consisting of ketoprofen, dexketoprofen, piroxicam, tenoxicam, and mixtures thereof, and the piperazine or ethylenediamine may optionally be filtered with a suitable filtering device to remove small particulate matter. For example, the solution may be filtered with a 0.22 micron syringe filter. Of course, other filtering devices and methods may be used, and may be more appropriate depending on the amount of the composition being prepared.

In another embodiment, the composition comprising the selected NSAID, preferably one or more NSAIDs selected from the group consisting of ketoprofen, dexketoprofen, piroxicam, tenoxicam, and mixtures thereof, and the piperazine or ethylenediamine can be prepared using a double decomposition method, for example, the reaction of a salt, such as sodium salt, of the selected NSAID acid and the hydrochloride of ethylenediamine or piperazine. Preferably, when a double decomposition method is employed, a solvent which has low water content is used in order to facilitate precipitation of the by-product salt, e.g., sodium chloride, formed by the reaction so that it can be removed by filtration.

In certain embodiments, the liquid compositions, in particular the liquid compositions used for injection, can be prepared by dissolving the solid NSAID-piperazine or the NSAID-ethylenediamine composition in an appropriate liquid carrier.

Moreover, the preparation of the liquid formulation for injection, from the reaction of the salt of the NSAID, preferably one or more NSAIDs selected from the group consisting of ketoprofen, dexketoprofen, piroxicam, tenoxicam, and mixtures thereof, with piperazine hydrochloride or ethylenediamine hydrochloride, can be carried out directly in the finished injection solution, rather than first isolating the NSAID-piperazine or NSAID-ethylenediamine complex formed, provided that the sodium chloride formed in this direct method does not exceed physiologically acceptable levels.

In another embodiment, the process of the invention is directed to the in situ preparation of the complex of the invention, as described above. In certain aspects, in situ preparation of the complex comprises adding one or more selected NSAIDs, in racemic, enantiomeric excess, or enantiomeric form, preferably selected from the group consisting of ketoprofen, dexketoprofen, piroxicam, tenoxicam, and mixtures thereof; to an aqueous solution comprising a compound selected from ethylenediamine, piperazine, and mixtures thereof. For example, an amount of the NSAID, either as a solid or in a solution, can be added to a vessel, for example a glass vessel, which contains a liquid solution comprising ethylenediamine, piperazine, or mixtures thereof. During and/or after the addition of the NSAID, the resulting solution is mixed for a period of time until the NSAID and ethylenediamine or piperazine are in solution and/or complexed. The liquid solution may further contain suitable pharmaceutical excipients, buffers, preservatives, and the like, such that the resulting solution after mixing is ready for use in the methods described herein. In one respect, this in situ process is advantageous for the production of an aqueous pharmaceutical composition of the present invention over a process which first requires the manufacture, characterization, and release of a solid complex according to the invention, and shipment to the site of manufacture of the finished dosage form, wherein said solid NSAID-piperazine or NSAID-ethylenediamine complex is then dissolved in an appropriate aqueous carrier. Various amounts of the NSAID and base can be used in the in situ process to make a composition as described throughout the present application. For example, suitable amounts can be used to prepare a pharmaceutical composition according to the present invention wherein the ratio, concentration, amount, pH, etc. are specified herein.

In an alternative fashion, the in situ process may comprise the addition of ethylenediamine, piperazine, or mixtures thereof, in solution or not, to an appropriate container, for example, a glass-lined or stainless steel mixing vessel, containing an aqueous carrier, e.g., WFI, and an NSAID selected from ketoprofen, dexketoprofen, tenoxicam, and piroxicam.

For example, an in situ process of preparing the composition of the claimed invention can be used to prepare a composition comprising: (a) one or more selected NSAIDs, in racemic, enantiomeric excess, or enantiomeric form, preferably selected from the group consisting of ketoprofen, dexketoprofen, piroxicam, and mixtures thereof; (b) a compound selected from ethylenediamine, piperazine, tenoxicam, and mixtures thereof; (c) sterile water; (d) one or more pharmaceutically acceptable buffers; and (e) optionally one or more pharmaceutically acceptable preservatives; and wherein the pH is from about 6.5 to about 8.5, preferably from about 7.0 to about 8.0; the ratio of said NSAID to said compound is about 3:1 to about 1:3, preferably from about 2:1 to about 1:2; and the concentration of said NSAID is about 0.1 mg/mL to about 100 mg/mL, preferably about 1, 5, 10, 20, 25, 40, 50, 60, or 75 mg/mL.

### Determination of Analgesic Activity

The analgesic effects of the compositions of the present invention can be evaluated in one or more of the tests described below:

### Rat Tail Flick Test

The tail flick test was first described by D'Amour and Smith (1941), and remains essentially unchanged in application. (See generally D'Amour, F. E. and Smith, D. L., “A method for determining loss of pain sensation”, J. Pharmacol. Exp. Therap., 72:74-79 (1941); Dewey, D. L. and Harris, L. S., The Tail-flick test. In: S. Ehrenpreis and A. Neidle (Eds.), Methods in Narcotic Research, Marcel Dekker, Inc., New York, 1975, pp. 101-109; and Dubner, R. and Ren, K., “Assessing transient and persistent pain in animals.” In: P. D. Wall and R. Melzack (Eds.), Textbook of Pain, Churchill Livingstone, London, 1999, pp. 359-369). Quite simply, the tail of a rat or mouse is exposed to radiant heat, and the latency to withdraw is determined. The basal heat intensity is set so that naïve rats withdraw their tails within 2 to 3 sec. A cut-off latency of 10 sec (i.e., 3 to 4 times basal control value) is commonly employed to prevent tissue damage. An alternative to using radiant heat is to dip the tail into a water bath maintained at a fixed temperature, usually in the moderately noxious range of about 52° C. or 55° C. One advantage of a water bath is that the temperature is kept constant.

The tail-flick test is considered to be very robust in that weak analgesic agents are not detected by this test. In contrast, it is considered highly selective. There is a high degree of correlation between drugs that are identified as antinociceptive in the tail-flick test and clinically active analgesic agents. It is especially predictive of rank-order of potency of opioid-type analgesic agents, and the clinically effective dose of a novel opioid may be predicted by the relative potency of the drug to a known substance, such as morphine, based on this assay. Importantly, agents that are sedating and may produce a positive response in the writhing test or hot plate test do not show antinociceptive activity in the tail-flick test. It is even possible to perform the tail-flick test in lightly anesthetized animals.

Data obtained from the rat tail-flick test conform to a graded dose-response curve. The raw tail withdrawal latencies are converted to a % MPE (% maximal possible effect) by the formula:

% MPE=100×(test latency−basal latency)/(cut-off−basal latency).

This formula constrains the data to fit between 0% MPE and 100% MPE. This allows the generation of dose-response curves and the calculation of ED₅₀ values (50% effective doses) with attendant confidence intervals. These calculations then allow for the determination of relative potencies of different drugs and allow for the isobolographic determination of possible synergistic effects. Instances where the test latency is less than the basal latency produces a negative % MPE, which is meaningless unless one is measuring hyperalgesia. By convention, these values are set to 0% MPE when the expected drug effect is antinociception or no activity.

### Carrgeenan-Induced Inflamed Paw Model

Models of inflammation that produce more persistent pain include the injection of carrageenan into the footpad of the limb; the potential analgesic and/or anti-inflammatory properties of putative analgesics substances can be evaluated in this model. (See generally Bhalla T. N. & Tangri, K. K., “The time course of the carrageenan-induced oedema of the paw of the rat,” J. Pharm. Pharmacol. 22:721 (1970); Randall, L. O. & Selitto, J. J., “A method for measurement of analgesic activity on inflamed tissue,” Arch. Int. Pharmacodyn. Ther. 111:409-419 (1957); Hargreaves, K., et al. “A new and sensitive method for measuring thermal nociception in cutaneous hyperalgesia,” Pain 32:77-88 (1988)).

Typically, rats are handled and acclimatized to the behavioral testing equipment over a minimum of 2 days prior to testing. Behavioral tests are performed on all rats on the day prior to dosing to establish baseline values, and the animals are randomized into treatment groups based on these pre-dose responses. An assessment of the inflammatory agent (carrageenan) is performed prior to the main study, using the chosen behavioral tests. On the day of dosing, an inflammatory response is induced in the left hind paw of each rat by an intraplantar injection (approx. 0.05 mL) of carrageenan (0.6% w/v), under brief anesthesia. The test substance, reference substance, or vehicle is generally administered 30 minutes prior to carrageenan administration for oral dosing.

The following tests may be performed. A minimum period of 5 minutes is allowed between each type of test (or repeat challenges to the same paw) to reduce the risk of sensitization.

Paw Volume: Each animal is gently restrained, their hind limb extended, and the paw placed in the pre-filled chamber of a Digital Plethysmometer. The paw volume is then calculated based on the volume of liquid displaced in the chamber, for both the ipsilateral and contralateral hind paws.

Mechanical hyperalgesia test: Each rat is gently restrained, its hind limb extended, and the paw placed lightly on the Randall-Selitto device. A progressively increasing pressure is then applied to the dorsal surface of the paw via a blunt peg attached to a weight level, and the withdrawal threshold calculated for both the ipsilateral and contralateral hind paws. The maximum pressure applied is about 250 g. The withdrawal threshold is defined as the minimum force (in grams) required to elicit a reflex withdrawal response. Typical end points are a struggle response, paw withdrawal or a squeak response.

Thermal hyperalgesia test: Rats are placed in clear plastic chambers with a glass floor and allowed a short period to acclimatize to their environment prior to testing (approximately 2-5 minutes). The animals are then challenged with a radiant infrared heat source, directed at the plantar surface of their hind paw from below, and the withdrawal latency calculated for both the ipsilateral and contralateral hind paw.

Standard statistical methods are employed to evaluate test substance related effects. Data are analyzed for homogeneity and either parametric or non-parametric methods applied.

### Kim and Chung Model

Dose-response curve against tactile hyperesthesia and thermal hyperalgesia caused by peripheral nerve injury are generated. The peripheral nerve injury is established by tight ligation of the L5 and L6 spinal nerves, according to the techniques established by Chung and colleagues (Kim and Chung Pain, 1992: 50, 355-363.). Spinal nerve ligation (SNL) reliably produces tactile hyperesthesia and thermal hyperalgesia in rats. Tactile hyperesthesia is widely accepted as a model of allodynia to light touch often reported by patients with nerve injury. Thermal hyperalgesia represents a model of enhanced sensitivity to pain. The standard protocol for the evaluation of tactile hyperesthesia is to determine the paw withdrawal threshold of the hindpaw of the rats in response to probing with von Frey filaments. Thermal hyperalgesia is indicated by a significant reduction in paw withdrawal latency to noxious radiant heat projected onto the plantar aspect of the hindpaw of the rat. Tests are conducted with systemic (SC) and intrathecal (IT) administration. In order to properly conduct this study, sham-operated animals are also required. Since sham-operated rats do not develop tactile hyperesthesia, only responses to thermal stimuli are tested. Testing of sham-operated rats requires an additional 48 animals. Approximately 26 rats are be used for initial dose-finding experiments. Animals are tested within 7 to 10 days of SNL. Surgery and testing may be staggered to optimize testing efficiency. The intrathecal drug administration studies require implantation of catheters 7 days prior to the SNL surgery, and testing at 10 days after SNL surgery.

### Third Molar Extraction Model

Male and female patients with acute postsurgical pain following the removal of one or more bony impacted third molars are participants. Within 4 to 6 hours after completion of surgery, patients who are experiencing moderate or severe pain, as measured by a visual analog pain intensity scale (VAS≧50 mm) and by a categorical pain intensity scale (moderate or severe pain descriptor), and who meet all other inclusion/exclusion criteria are admitted to the study. Patients are randomly assigned to receive drug or placebo. Pain intensity (VAS and categorical), pain relief (categorical) and whether pain is half-gone is recorded by the patient under the supervision of the investigator or study coordinator at the various time points: Baseline (0 hour—pain intensity only), 15, 30 and 45 minutes, and at 1, 1.5, 2, 3, 4, 5, 6, 7, 8 and 12 hours after administration of study medication, and immediately prior to the first rescue dose. Time to onset of perceptible and meaningful pain relief is evaluated using the two stopwatch method. Patients record their global evaluation of study medication at the completion of the 8-hour assessment or at the time of first rescue medication use. Efficacy endpoints include Total Pain Relief (TOTPAR), Sum of Pain Intensity Difference (SPID) and Sum of Pain Relief Intensity Difference (SPRID) at various time points, Time to First Rescue, Time Specific Pain Intensity Difference (PID), Time Specific Pain Relief (PR), Peak Pain Intensity Difference (PPID), Peak Pain Relief (PPR), Time to Confirmed Perceptible Pain Relief (stopwatch) and Time to Meaningful Pain Relief (stopwatch) and Patient Global Evaluation.

### Bunionectomy Surgery Model

Male or female patients requiring primary unilateral first metatarsal bunionectomy surgery alone or with ipsilateral hammertoe repair (without additional collateral procedures) under regional anesthesia (Mayo block) are participants.

Patients who experience moderate or severe pain on a categorical scale (moderate or severe descriptor) and on a visual analog pain intensity scale (VAS; 50 mm) within 6 hours following completion of bunionectomy surgery are randomly assigned to receive study drugs or placebo. Patients are encouraged to wait at least 60 minutes before requesting remedication for pain. At the completion of the single-dose phase (8 hours) or at first request for remedication (whichever is earlier), patients enter into a multiple-dose phase lasting approximately 72 hours. During the multiple dose phase, patients receive study medication or placebo at a fixed dose interval (e.g., every 8, 12 or 24 hours). Once the multiple dose phase of the study has begun, patients experiencing pain between scheduled doses of study medication are provided access to supplemental open-label (rescue) analgesia. Patients whose pain cannot be adequately managed on a combination of study medication and rescue medication or who develop unacceptable side effects during the study are discontinued from further study participation and their pain managed conventionally.

Pain intensity (VAS and categorical), pain relief (categorical) and whether pain is half-gone is recorded by the patient under the supervision of the investigator or study coordinator at representative time points, e.g., Baseline (pain intensity only), 15, 30 and 45 minutes and 1, 1.5, 2, 3, 4, 5, 6, 7 and 8 hours after administration of study medication and immediately prior to the first remedication. Time to onset of perceptible and meaningful pain relief is evaluated using the double-stopwatch method. Patients complete a global evaluation of study medication at the completion of the 8-hour assessment or just prior to the first remedication. Following completion of the single-dose phase (8 hours or just prior to first remedication, if ≦8 hours), patients begin the multiple dose phase of the study. During the multiple dose phase, patients record their overall pain intensity since the previous scheduled dose, their current pain intensity and a patient global, immediately prior to each scheduled dose of study medication and at early termination.

Measures of efficacy in the single-dose phase include Sum of Pain Intensity Difference (SPID), Total Pain Relief (TOTPAR), Sum of Pain Relief Intensity Difference (SPRID), Time to First Remedication, Time Specific Pain Intensity Difference (PID), Time Specific Pain Relief (PR), Peak Pain Intensity Difference (PPM), Peak Pain Relief (PPR), Time to Confirmed Perceptible Pain Relief (stopwatch) and Time to Meaningful Pain Relief (stopwatch) and Patient Global Evaluation. Measures of efficacy in the multiple-dose phase include the time specific overall pain intensity, current pain intensity and patient global at the time of scheduled remedication, the average of overall pain intensity, current pain intensity and patient global over 0-24, 24-48 and 48-72 and number of doses of rescue analgesic over 0-24, 24-48 and 48-72 and 0-72 hours.

The included examples are illustrative but not limiting of the methods and composition of the present invention. Other suitable modifications and adaptations of the variety of conditions and parameters normally encountered and obvious to those skilled in the art are within the spirit and scope of the invention.

### EXAMPLES

### Example 1

### Ketoprofen-Piperazine (1:2) Complex

A aqueous mixture of 0.5 M of ketoprofen and 1.0 M and piperazine was sonicated until a clear solution was obtained. The solution was then filtered with a 0.45 pin syringe filter (pH about 10.00 for filtered aliquot). Small aliquots (about 0.250 mL) were placed in a 12-depression spot ceramic plate. The physical mixture precipitated out at ambient temperature over several days (2-3 days).

The ketoprofen-piperazine complex was formed as a semisolid (physical mixture) having an aqueous solubility of greater than 360 mg/mL and a pH of about 9.4 (for a saturated solution).

### Example 2

### Ketoprofen-Ethylenediamine (1:2) Complex

A mixture of 0.5 M of ketoprofen and 1.0 M and ethylenediamine was sonicated until a clear solution was obtained. The solution was then filtered with a 0.45 μm syringe filter (pH about 10.00 for filtered aliquot). Small aliquots (about 0.250 mL) were placed in a 12-depression spot ceramic plate. The physical mixture precipitated out at ambient temperature over several days (2-3 days).

The ketoprofen-ethylenediamine complex was formed as a semisolid having an aqueous solubility of greater than 500 mg/mL and a pH of about 7.3 (for a saturated solution).

### Example 3

### Static Dilution Test of a Ketoprofen-Ethylenediamine Composition

A 500 mg ketoprofen solution was serially diluted in normal saline (NS) and isotonic Sorensen phosphate buffer pH 7.4 (ISPB) separately. Dilutions were made by adding respective volumes of the solutions to NS or ISPB such that a range of 4-2560 fold dilution was covered. Thus, for the first dilution 1 part of solution was added to 3 parts of NS or ISPB resulting in a 4-fold dilution. Subsequent dilutions were made serially resulting in a final 8-, 16-, 32-, 64-, 128-, 256-, and 2560-fold dilution. After every dilution, vials were vortexed and allowed to stand for 1 minute. Presence of precipitation in the form of turbidity or cloudiness was tested visually using Laser Diffraction (Tyndall Effect). The pH was measured after every dilution. Vials were rechecked for the presence of precipitation after 24 hours.

Ketoprofen-ethylenediamine passed the static dilution test. No precipitation or turbidity was observed visually upon passing laser beam for the entire range of dilution (4-2560 fold) both after 1 minute as well as 24 hours following dilution.

Results of Static Dilution Studies

| | | Normal Saline | | | ISBP pH 7.4 | | |
|---|---|---|---|---|---|---|---|
| | Ketoprofen | | Initial | 24 Hr | | Initial | 24 Hr |
| Dilution | [mg/ml] | pH | Appearance | Appearance | pH | Appearance | Appearance |

500 mg/mL + 1M Ethylenediamine (2:1) in DI Water, ISBP

| | | | | | | | |
|---|---|---|---|---|---|---|---|
| 4 | 125.00 | 7.39 | clear | clear | 7.44 | clear | clear |
| 8 | 62.50 | 7.22 | clear | clear | 7.31 | clear | clear |
| 16 | 31.25 | 7.03 | clear | clear | 7.25 | clear | clear |
| 32 | 15.63 | 6.94 | clear | clear | 7.29 | clear | clear |
| 64 | 7.81 | 6.9 | clear | clear | 7.33 | clear | clear |
| 128 | 3.91 | 6.88 | clear | clear | 7.37 | clear | clear |
| 256 | 1.95 | 6.87 | clear | clear | 7.38 | clear | clear |
| 2560 | 0.20 | 6.85 | clear | clear | 7.4 | clear | clear |

250 mg/mL + 0.5M Ethylenediamine (2:1) in DI Water, ISBP

| | | | | | | | |
|---|---|---|---|---|---|---|---|
| 4 | 62.50 | 7.2 | clear | clear | 7.31 | clear | Clear |
| 8 | 31.25 | 7.05 | clear | clear | 7.25 | clear | Clear |
| 16 | 15.63 | 6.94 | clear | clear | 7.29 | clear | Clear |
| 32 | 7.81 | 6.9 | clear | clear | 7.35 | clear | Clear |
| 64 | 3.91 | 6.89 | clear | clear | 7.37 | clear | Clear |
| 128 | 1.95 | 6.88 | clear | clear | 7.39 | clear | Clear |
| 256 | 0.98 | 6.87 | clear | clear | 7.4 | clear | Clear |
| 2560 | 0.10 | 6.87 | clear | clear | 7.4 | clear | Clear |

### Example 4

### Static Dilution Test of a Ketoprofen-Piperazine Composition

A 50 mg ketoprofen solution was serially diluted in normal saline (NS) and isotonic Sorensen phosphate buffer pH 7.45 (ISPB) separately. Dilutions were made by adding respective volumes of the solutions to NS or ISPB such that a range of 2-3200 fold dilution was covered. Thus, for the first dilution 1 part of solution was added to 1 parts of NS or ISPB resulting in a 2-fold dilution. Subsequent dilutions were made serially resulting in a final 4-, 8-, 16-, 32-, 320-, 3200-fold dilution. After every dilution vials were vortexed and allowed to stand for 1 minute. Presence of precipitation in the form of turbidity or cloudiness was tested visually using Laser Diffraction (Tyndall Effect). The pH was measured after every dilution. Vials were rechecked for the presence of precipitation after 24 hours.

Ketoprofen-piperazine passed the static dilution test, i.e., no precipitation or turbidity was observed visually upon passing laser beam for the entire range of dilution (4-3200 fold) both after 1 minute as well as 24 hours following dilution.

Results of Static Dilution Studies

| | | Normal Saline | | | ISBP pH 7.45 | | |
|---|---|---|---|---|---|---|---|
| | Ketoprofen | | Initial | 24 Hr | | Initial | 24 Hr |
| Dilution | [mg/ml] | pH | Appearance | Appearance | pH | Appearance | Appearance |

50 mg/ml + 0.2M Piperazine (1:1) in DI Water, ISBP

| | | | | | | | |
|---|---|---|---|---|---|---|---|
| — | 50.000 | 8.39 | clear | Clear | 8.39 | clear | Clear |
| 2 | 25.000 | 8.29 | clear | Clear | 7.70 | clear | Clear |
| 4 | 12.500 | 8.24 | clear | Clear | 7.42 | clear | Clear |
| 8 | 6.250 | 8.21 | clear | Clear | 7.31 | clear | Clear |
| 16 | 3.125 | 8.13 | clear | Clear | 7.25 | clear | Clear |
| 32 | 1.563 | 8.08 | clear | Clear | 7.23 | clear | Clear |
| 320 | 0.156 | 6.94 | clear | Clear | 7.20 | clear | Clear |
| 3200 | 0.016 | 6.53 | clear | Clear | 7.20 | clear | Clear |

12.7 mg/ml + 0.2M Piperazine (1:2) in DI Water, ISBP

| | | | | | | | |
|---|---|---|---|---|---|---|---|
| — | 12.700 | 9.87 | clear | Clear | 9.87 | clear | Clear |
| 2 | 6.350 | 9.86 | clear | Clear | 9.69 | clear | Clear |
| 4 | 3.175 | 9.84 | clear | clear | 9.16 | clear | Clear |
| 8 | 1.588 | 9.80 | clear | clear | 7.75 | clear | Clear |
| 16 | 0.794 | 9.74 | clear | clear | 7.42 | clear | clear |
| 32 | 0.397 | 9.56 | clear | clear | 7.31 | clear | clear |
| 320 | 0.040 | 8.33 | clear | clear | 7.21 | clear | clear |

### Example 5

### Dynamic Dilution Test

Two 50 mg/mL ketoprofen formulations (ketoprofen-ethylenediamine and ketoprofen-piperazine) were tested and compared for the occurrence of precipitation upon dynamic dilution in ISPB using a validated standard dynamic injection apparatus with high predictive value for phlebitis. (See Yalkowsky, S. H. et al., J. Pharma. Sci. 72:1014-19 (1983); Johnson et al., J. Pharm. Sci. 92:1574-81 (2003)). Each solution at a flow rate of 5 mL/min was introduced to ISPB flowing at a rate of 5 mL/min in a tube for 30 seconds and passed through a flowcell. Absorbance was measured at 540 nm using a UV spectrophotometer. The wavelength 540 nm was chosen since all the formulations had no or very small absorbance at that wavelength. The results of the tests are shown in the Figures.

The results indicate that there was no substantial precipitation of the formulations in this test. The solutions, tested at 50 mg/mL, both passed the dynamic precipitation test, i.e., no significant change in absorbance was observed at 540 nm. The minor absorbance (<0.1) was neglected as this was considered to result from the schlieren pattern developed during mixing due to difference in viscosity of the solution and ISPB. Phenyloin solution, which is known to produce phlebitis, was used as a positive control in this test. Note also that the sharp spikes observed for the test formulations are the result of schlieren patterns that result from density differences between the formulation and the diluent and are not due to precipitation.

### Example 6

### Relative Viscosity of Ketoprofen-Ethylenediamine Formulation

The relative viscosity of the ketoprofen-ethylenediamine solutions (50 and 500 mg/mL) and ketoprofen-piperazine solutions (12.7 mg/mL and 50 mg/mL) were determined by the capillary method using an Ostwald-Cannon-Fenske viscometer. The time required for each solution to pass between two marks on the viscometer was used to determine the relative velocity. Density was measured separately for the solutions. Water was used as reference liquid (viscosity of water at 20° C. is 1.002 cP). The following equation was used to calculate the viscosity:

$${η}_{1} / {η}_{2} = {ρ}_{1} {t}_{1} / {ρ}_{2} {t}_{2}$$

where:
η₁ is the viscosity of test solution
η₂ is the viscosity of water at 20° C.
ρ₁ is the density of test solution
ρ₂ is the density of water
t₁ is the time taken by test solution to pass between two marks in the viscometer
t₂ is the time taken by water to pass between two marks in the viscometer

The viscosities of ketoprofen-ethylenediamine 50 mg/mL and 500 mg/mL, as determined by the Ostwald-Cannon-Fenske viscometer were 1.2 and 26 cP, respectively.

The viscosities of ketoprofen-piperazine solutions 12.7 and 50 mg/Ml were 1.12 Cp and 1.25 cP, respectively.

### Example 7

### Ionic Strength of Ethylenediamine and Piperazine Salts of Ketoprofen

The ionic strength for ketoprofen-ethylenediamine 50 and 500 mg/mL and ketoprofen-piperazine 12.7 and 50 mg/mL solutions were calculated using following equation:

$$μ = 0.5 Σ {{c}_{i} ( {z}_{i} )}^{2}$$

where:
μ is the ionic strength of the solution
cᵢ is the concentration of the ionic species
zᵢ is the charge on the ionic species

The ionic strength of ketoprofen-ethylenediamine 50 and 500 mg/mL was 0.295 and 2.95, respectively.

The ionic strength of ketoprofen-piperazine 12.7 and 50 mg/mL solutions was 0.225 and 0.5, respectively

### Example 8

The purpose of this study was to evaluate the thrombophlebitis potential of unsalified ketoprofen relative to its ethylenediamine and piperazine salts, using a dynamic injection model of phelebitis. Unsalified ketoprofen 50 mg/mL was prepared in 0.1 N NaOH. The sample was sonicated for approximately 20 minutes and the resulting suspension of pH 6.50 was filtered with a 0.45 μm syringe filter and tested for the occurrence of precipitation upon dynamic dilution in ISPB using a validated standard dynamic injection apparatus with high predictive value for phlebitis. Ketoprofen at a flow rate of 5 mL/min was introduced to ISPB flowing at a rate of 5 mL/min in a tube for 30 seconds and passed through a flowcell. Absorbance was measured at 540 nm using a UV spectrophotometer. The wavelength 540 nm was chosen since all the formulations had no or very small absorbance at that wavelength. The results were compared with ketoprofen-ethylenediamine and ketoprofen-piperazine (without any pH adjustment or additives).

The absorbance from the unsalified pH adjusted ketoprofen was a 83-fold and 445-fold greater than the relative absorbencies of ethylenediamine and piperazine salts, respectively, at a wavelength of 540 nm.

| | Max UV-Absorbance (540 nm) | | |
|---|---|---|---|
| Sample | Injection-1 | Injection-2 | Injection-3 |
| Ketoprofen 50 mg/mL | 2.7763 | 3.1730 | 3.1730 |
| suspension pH-6.50 | | | |
| Ketoprofen-Ethylenediamine | 0.0413 | 0.0295 | 0.0383 |
| 50 mg/mL | | | |
| Ketoprofen-Piperazine | 0.0152 | 0.0030 | 0.0023 |
| 50 mg/mL (1:2 mix) | | | |

### Example 9

The purpose of this study was to test the stability of ethylenediamine and piperazine salts of ketoprofen. Ketoprofen as the ethylenediamine salt (0.1 M) at an initial concentration of 50 mg/mL and ketoprofen as the piperazine crystalline salt at an initial concentration of 10 mg/mL were retested after 3 months storage at room temperature using an HPLC analytical method. There was no significant degradation of ketoprofen. The detected concentration of ketoprofen after three months was 49.4 mg/mL (98.8%) and 9.94 mg/mL (99.4%) for ketoprofen-ethylenediamine and ketoprofen-piperazine, respectively, which is within the range of experimental error.

### Example 10

The purpose of this study was to assess the acute irritative potential of two dose levels of unsalified ketoprofen using a validated in vivo model for assessment of venous irritation and thrombophlebitis. Ketoprofen solution at a concentration of 50 mg/mL was prepared in 0.1 N NaOH. The pH was gradually increased over 4 hours in order to avoid the presence of excess sodium hydroxide. The pH was increased using solid sodium hydroxide. The final pH of the solution was 8.72 (constant over 12 hours). The solution was filtered with a 0.45 μm syringe filter into a sterile evacuated vial.

Two male, purpose-bred New Zealand White rabbits (age 22 weeks, weight 2.8 to 3.2 kilograms) were used for this study. Animals were individually housed, with a 12 hours light/12 hours dark cycle. All animals had access to Harlan Teklad Hi-Fiber Rabbit Diet. Tap water was available ad libitum. Study animals were acclimated to their housing for a minimum of 5 days prior to dosing. Prior to injection, the hair surrounding all injection sites was clipped and the sites were swabbed with 70% ethanol or isopropyl alcohol. One rabbit each was administered test article, Treatment A (Ketoprofen mg/0.1 mL or Treatment B (Ketoprofen 15 mg/0.3 mL) intravenously into a marginal vein of each ear, over one minute (i.e., Ketoprofen 5 mg/0.1 mL over one minute or Ketoprofen 15 mg/0.3 mL over one minute). All animals were treated on Day 1 of the study. The test sites were identified with indelible marker. Intravenous sites were marked with an indelible marker at the area of entry of the needle, approximately at the end of its progress and approximately 1 cm from that point.

Clinical observations for any pharmacotoxicological signs were recorded and irritation at injection sites was scored, using the Draize evaluation score.

The intravenous administration of Treatment A (Ketoprofen: 5 mg/0.1 mL) resulted in well defined erythema and slight edema were observed at the 24 hour observation period. The intravenous administration of Treatment B (Ketoprofen: 15 mg/0.3 mL) resulted in very slight to moderate to severe erythema and very slight edema at the 24 hour observation period.

Under the conditions of this study, intravenous administration of Treatment A (Ketoprofen: 5 mg/0.1 mL) and Treatment B (Ketoprofen: 15 mg/0.3 mL) produced irritation in the ear veins of rabbits. The irritation was progressive over time and displayed the potential for a dose related response.

### Example 11

The purpose of this study was to assess the acute irritative potential of unsalified NSAIDs versus their respective ethylenediamine and piperazine salts when administered intravenously to rabbits, using a validated model for assessment of venous irritation and thrombophlebitis and a randomized design.

Purpose-bred New Zealand White rabbits (age 8-24 weeks, weight 2.0 to 3.5 kilograms) were used for this study. Animals were individually housed, with a 12 hours light/12 hours dark cycle. All animals had access to Harlan Teklad Hi-Fiber Rabbit Diet. Tap water was available ad libitum. Study animals were acclimated to their housing for a minimum of 5 days prior to dosing. Prior to injection, the hair surrounding all injection sites was clipped and the sites were swabbed with 70% ethanol or isopropyl alcohol.

Test articles consisted of unsalified ketoprofen, unsalified dexketoprofen, unsalified piroxicam, ketoprofen-ethylenediamine, dexketoprofen-ethylenediamine, piroxicam-ethylenediamine, ketoprofen-piperazine, and dexketoprofen-piperazine. Eight rabbits each were allocated to each group as follows:

| Number of | | |
|---|---|---|
| Animals | Treatment | Dose |
| 8 | Piroxicam - Unsalified | 1.25 mL of 10 mg/mL |
| 8 | Piroxicam - Ethylenediamine | 1.25 mL of 10 mg/mL |
| 8 | Ketoprofen - Unsalified | 1.25 or 0.8 mL of 50 mg/mL |
| 8 | Dexketoprofen - Unsalified | 1.25 or 0.8 mL of 25 mg/mL |
| 8 | Dexketoprofen - | 1.25 of 25 mg/mL-1.25 mL |
| | Ethylenediamine | |
| 8 | Ketoprofen - Piperazine | 1.25 mL of 50 mg/mL |
| 8 | Dexketoprofen - Piperazine | 1.25 of 25 mg/mL |
| 8 | Ketoprofen - Ethylenediamine | 1.25 mL of 50 mg/mL |

Eight rabbits per group were anesthetized prior to test article administration with a combination of ketamine and xylazine to reduce possible injection site trauma. Puralube Vet ointment was placed in each eye after subcutaneous anesthesia was administered. All rabbits were administered the respective test article intravenously into a marginal vein of each ear, over one minute. Intravenous sites were marked with an indelible marker at the area of entry of the needle, approximately at the end of its progress and approximately 1 cm from that point, at the time of injection and as needed throughout the duration of the study. Clinical observations were recorded at 1, 4, and 24 hours. Irritation at injection sites was scored at 1 and 24 hours, using the Draize evaluation score. Injection sites underwent histopathological evaluation. Treatment differences in erythema and edema scores following treatment were compared for the unsalified NSAID with their respective ethylenediamine and piperazine salts using the Wilcoxon Signed-Ranks test.

The injection site erythema and edema scores for unsalified ketoprofen, dexketoprofen and piroxicam were compared with their respective ethlyenediamine and piperazine salts (ethylenediamine salt only for piroxicam).

Piroxicam vs. Piroxicam-Ethylenediamine: At the 1-hour post-infusion time point, piroxicam-ethylenediamine was not statistically different from unsalified piroxicam on the erythema (p<0.38) and edema (p<0.38) scores. At the 24-hour post-infusion time point, piroxicam-ethylenediamine produced significantly lower erythema (p<0.0001) and edema (p<0.00006) scores than unsalified piroxicam.

Ketoprofen vs. Ketoprofen-Ethylenediamine: At the 1-hour post-infusion time point, ketoprofen-ethylenediamine produced significantly lower erythema (p<0.0002) and edema (p<0.0002) scores than unsalified ketoprofen. Similarly, at the 24-hour post-infusion time point, ketoprofen-ethylenediamine produced significantly lower erythema (p<0.0002) and edema (p<0.0008) scores than unsalified ketoprofen.

Ketoprofen vs. Ketoprofen-piperazine: At the 1-hour post-infusion time point, ketoprofen-piperazine produced significantly lower erythema (p<0.0002) and edema (p<0.0003) scores than unsalified ketoprofen. Similarly, at the 24-hour post-infusion time point, ketoprofen piperazine produced significantly lower erythema (p<0.0003) and edema (p<0.0001) scores than unsalified ketoprofen.

Dexketoprofen vs. Dexketoprofen-Ethylenediamine: At the 1-hour post-infusion time point, dexketoprofen-ethylenediamine produced significantly lower erythema (p<0.0001) and edema (p<0.0003) scores than unsalified dexketoprofen. Similarly, at the 24-hour post-infusion time point, dexketoprofen-ethylenediamine produced significantly lower erythema (p<0.0006) and edema (p<0.021) scores than unsalified dexketoprofen.

Dexketoprofen vs. Dexketoprofen-piperazine: At the 1-hour post-infusion time point, dexketoprofen-piperazine produced significantly lower erythema (p<0.0019) and edema (p<0.0019) scores than unsalified dexketoprofen. Similarly, at the 24-hour post-infusion time point, dexketoprofen-piperazine produced significantly lower erythema (p<0.0002) and edema (p<0.0024) scores than unsalified dexketoprofen.

The mean injection site erythema and edema scores for unsalified NSAID and their respective piperazine and/or ethlyenediamine salts at 1 and 24 hours post infusion are presented in the Table below.

Mean Injection Site Erythema and Edema Score (Draize Score)

| | | 1 Hour Post-dose | | | | 24 Hour Post-dose | | | |
|---|---|---|---|---|---|---|---|---|---|
| | # of | Left Ear | | Right Ear | | Left Ear | | Right Ear | |
| Treatment | Animals | ER | ED | ER | ED | ER | ED | ER | ED |
| Piroxicam - | 8 | 2.125 | 0.25 | 2.625 | 0.75 | 2.5 | 1.125 | 2.375 | 1.125 |
| Unsalified | | | | | | | | | |
| Piroxicam - | 8 | 2.0 | 0.875 | 2.5 | 1.125 | 0.75 | 0 | 1.375 | 0.125 |
| Ethylenediamine | | | | | | | | | |
| Ketoprofen - | 8 | 3.0 | 1.625 | 2.875 | 1.625 | 3.0 | 1.5 | 3.0 | 1.5 |
| Unsalified | | | | | | | | | |
| Ketoprofen - | 8 | 1.25 | 0 | 1.375 | 0 | 1.25 | 0 | 1.875 | 0.5 |
| Piperazine | | | | | | | | | |
| Ketoprofen - | 8 | 1.375 | 0.125 | 2 | 0.75 | 1.5 | 0.25 | 1.625 | 0.375 |
| Ethylenediamine | | | | | | | | | |
| Dexketoprofen - | 8 | 2.75 | 1.25 | 2.625 | 1.375 | 2.375 | 1.125 | 2.625 | 1.0 |
| Unsalified | | | | | | | | | |
| Dexketoprofen - | 8 | 1.25 | 0.125 | 1.375 | 0 | 0.75 | 0 | 1.0 | 0 |
| Ethylenediamine | | | | | | | | | |
| Dexketoprofen - | 8 | 2.0 | 0.375 | 1.875 | 0.625 | 1.125 | 0 | 1.75 | 0.375 |
| Piperazine | | | | | | | | | |

ER = Erythema;
ED = Edema

Microscopic observations of injection sites included congestion, edema, hemorrhage, inflammatory cell infiltrate, necrosis/loss of cellular detail (vessel and regional), thrombosis, necrosis/exudative scabs of the epithelium and occasional incidental observations of keratin cyst, acanthosis, dermal fibrosis or chronic dermal inflammation. Thrombosis was recorded for amorphous aggregates of fibrin like material within vascular lumens and variably adherent to vessel walls. These aggregates consistently lacked organization. Necrosis/exudative scabs at the epithelial surface were generally only recorded when they were proximal to the ear vein and hence likely to be representative of the needle point of entry through the epidermis.

Piroxicam vs. Piroxicam-Ethylenediamine: Piroxicam-ethylenediamine had a low incidence of inflammatory cell infiltrates at the end of needle progression and 1 cm away from the end of progression than unsalified piroxicam, which had an intermediate incidence. Similarly, piroxicam-ethylenediamine had none to negligible mean intensity (severity) of edema and infiltrates, compared with unsalified piroxicam which had an intermediate incidence.

Ketoprofen vs. Ketoprofen-Ethylenediamine: Ketoprofen-ethylenediamine had a low incidence of inflammatory cell infiltrates at the end of needle progression and 1 cm away from the end of progression than unsalified ketoprofen, which had a high incidence. Similarly, ketoprofen ethylenediamine had none to negligible mean intensity (severity) of edema and infiltrates, compared with unsalified ketoprofen which had a high incidence.

Ketoprofen vs. Ketoprofen-piperazine: Ketoprofen-piperazine had a low incidence of inflammatory cell infiltrates at the end of needle progression and 1 cm away from the end of progression than unsalified ketoprofen, which had a high incidence. Similarly, ketoprofen-piperazine had none to negligible mean intensity (severity) of edema and infiltrates, compared with unsalified ketoprofen which had a high incidence.

Dexketoprofen vs. Dexketoprofen-Ethylenediamine: Dexketoprofen-ethylenediamine had a low incidence of inflammatory cell infiltrates at the end of needle progression and 1 cm away from the end of progression than unsalified dexketoprofen, which had an intermediate incidence. Similarly, dexketoprofen-ethylenediamine had none to negligible mean intensity (severity) of edema and infiltrates, compared with unsalified dexketoprofen which had an intermediate incidence.

Dexketoprofen vs. Dexketoprofen-piperazine: Dexketoprofen-piperazine had a low incidence of inflammatory cell infiltrates at the end of needle progression and 1 cm away from the end of progression than unsalified dexketoprofen, which had an intermediate incidence. Similarly, dexketoprofen piperazine had none to negligible mean intensity (severity) of edema and infiltrates, compared with unsalified dexketoprofen which had an intermediate incidence.

Under the conditions of this study, injection site erythema and edema scores for unsalified ketoprofen, dexketoprofen, and piroxicam were higher than their respective piperazine and/or ethlyenediamine salts. Similarly, histopathologic examination revealed that the incidence of inflammatory cell infiltrates and the intensity of edema and infiltrates for unsalified ketoprofen, dexketoprofen, and piroxicam were higher than their respective piperazine and/or ethlyenediamine salts.

### Example 12

### Pharmaceutical Composition Comprising Ketoprofen and Piperazine

Water (700 mL), purified by reverse osmosis, is adjusted to pH 5 with 0.1 N HCl and heated to 35° C. piperazine 16.94 g is added with stirring. The solution is stirred for 5 minutes and ketoprofen 50 g is slowly added with vigorous stirring. The solution is stirred for 30 minutes at 35° C. A sample is removed, allowed to cool and the osmolarity measured. If necessary, sorbitol (in an amount calculated to bring the osmolality of the solution at final volume of 1000 mL to between 280-300 mOsm/L) is slowly added with stirring. The heat is removed and stirring is continued until room temperature is reached. Final volume adjustment is made with water and the solution is stirred for 15 minutes. The pH is further adjusted to bring the final pH between 6.5 and 8.5. The solution is optionally first filtered through a 0.45 micron filter and deoxygenated with nitrogen or deoxygenated and finally passed through 0.22 micron membrane filter into presterilized syringes under nitrogen atmosphere and aseptic conditions. The syringes are sealed under nitrogen. The solution contains 50.0±3.0 mg/mL ketoprofen, as the piperazine salt as determined by HPLC.

### Example 13

### Pharmaceutical Composition Comprising Ketoprofen and Ethylenediamine

Water (700 mL), purified by reverse osmosis, is adjusted to pH 5 with 0.1 N HCl and heated to 35° C. Ethylenediamine 6.01 g is added with stirring. The solution is stirred for 5 minutes and ketoprofen 50 g is slowly added with vigorous stirring. The solution is stirred for 30 minutes at 35° C. A sample is removed, allowed to cool and the osmolarity measured. If necessary, sorbitol (in an amount calculated to bring the osmolality of the solution at final volume of 1000 mL to between 280-300 mOsm/L) is slowly added with stirring. The heat is removed and stirring is continued until room temperature is reached. Final volume adjustment is made with water and the solution is stirred for 15 minutes. The pH is further adjusted to bring the final pH between 6.5 and 8.5. The solution is optionally first filtered through a 0.45 micron filter and deoxygenated with nitrogen or deoxygenated and finally passed through 0.22 micron membrane filter into presterilized syringes under nitrogen atmosphere and aseptic conditions. The syringes are sealed under nitrogen. The solution contains 50.0-11.0 mg/mL ketoprofen, as the ethylenediamine salt as determined by HPLC.

### Example 14

### Pharmaceutical Composition Comprising Dexketoprofen and Ethylenediamine

Water (700 mL), purified by reverse osmosis, is adjusted to pH 5 with 0.1N HCl and heated to 35° C. Ethylenediamine 3.01 g is added with stirring. The solution is stirred for 5 minutes and dexketoprofen 25 g is slowly added with vigorous stirring. The solution is stirred for 30 minutes at 35° C. A sample is removed, allowed to cool and the osmolarity measured. If necessary, sorbitol (in an amount calculated to bring the osmolality of the solution at final volume of 1000 mL to between 280-300 mOsm/L) is slowly added with stirring. The heat is removed and stirring is continued until room temperature is reached. Final volume adjustment is made with water and the solution is stirred for 15 minutes. The pH is further adjusted to bring the final pH between 6.5 and 8.5. The solution is optionally first filtered through a 0.45 micron filter and deoxygenated with nitrogen or deoxygenated and finally passed through 0.22 micron membrane filter into presterilized syringes under nitrogen atmosphere and aseptic conditions. The syringes are sealed under nitrogen. The solution contains 25.0±0.6 mg/mL dexketoprofen, as the ethylenediamine salt as determined by HPLC.

### Example 15

### Pharmaceutical Composition Comprising Piroxicam and Ethylenediamine

Water (1000 mL), purified by reverse osmosis is adjusted to pH 4.5 with 0.1 N HCl. A 700 mL portion of the acidified water is heated to 35° C. and ethylenediamine 0.93 g is added with stirring. The solution is stirred for 5 minutes and piroxicam 10 g is slowly added with vigorous stirring. The solution is stirred for 30 minutes at 35° C. The heat is removed and stirring is continued until room temperature is reached. The volume is adjusted to 1000 ml with the remainder of the acidified water and the solution is stirred for 15 minutes. The pH is further adjusted to bring the final pH between 6.5 and 8.5. The solution is deoxygenated with nitrogen and passed through 0.22 micron membrane filter into presterilized vials under nitrogen atmosphere and aseptic conditions. The fill volume of the vial is 2 mL. The vials are sealed under nitrogen. The solution contains 10.0 mg 0.5 mg/mL piroxicam, as the ethylenediamine salt as determined by a sensitive and specific HPLC method. Vials stored for 12 weeks at 5° C., 25° C./60% RH, 30° C./60% RH, 40° C./75% RH show no visible signs of crystallization or significant deviation in pH and osmolarity and contain 100%±5% original piroxicam content as determined by stability indicating HPLC.

### Example 16

### Pharmaceutical Composition Comprising Piroxicam and Ethylenediamine

Materials: 1) Water for Injection (WFI), USP, Q.S. to 1000 mL; 2) Piroxicam salt of ethylenediamine 10.93 grams; 3) Nitrogen gas, NF. The preparation is manufactured, protected from light and under cover of nitrogen gas throughout, which is bubbled into 800 mL aliquot of WFI for 20 minutes. Piroxicam ethylenediamine is slowly added with vigorous stirring for 40 minutes at 35° C. The heat is removed and stirring is continued until room temperature is reached. The volume is adjusted to 1000 ml with the remainder of WFI and stirred for 15 minutes. The pH is further adjusted to bring the final pH between 7.0 and 7.5. Using a pressurized source of nitrogen gas, the solution is filtered through a 0.22 μm cartridge and collected in a suitable staging vessel protected from exposure to ultraviolet light. The solution is filled into Type 12-mL ampoule, with pre- and post-nitrogen gas flush. The sealed ampoules are subjected to terminal heat sterilization. The solution contains 10.0 mg±0.5 mg/mL piroxicam ethylenediamine as determined by a sensitive and specific HPLC method. Ampoules stored for 12 weeks at 5° C., 25° C./60% RH, 30° C./60% RH, 40° C./75% RH show no visible signs of crystallization or significant deviation in pH and osmolarity and contain 100%±5% original piroxicam content as determined by stability indicating HPLC.

### Example 17

### Pharmaceutical Composition Comprising Ketoprofen Ethylenediamine

Materials: 1) Water for Injection (WFI), USP, Q.S. to 1000 mL; 2) Ketoprofen salt of ethylenediamine 56.01 grams; 3) Nitrogen gas, NF. The preparation is manufactured, protected from light and under cover of nitrogen gas throughout, which is bubbled into 700 mL aliquot of WFI for 30 minutes. Ketoprofen ethylenediamine is slowly added with vigorous stirring for 30 minutes. The volume is adjusted to 1000 ml with the remainder of WFI and stirred for 20 minutes. The pH is further adjusted to bring the final pH between 7.5 and 9.0. Using a pressurized source of nitrogen gas, the solution is filtered through a 0.22 μm cartridge and collected in a suitable staging vessel protected from exposure to ultraviolet light. The solution is filled into Type 12-mL ampoule, with pre- and post-nitrogen gas flush. The sealed ampoules are subjected to terminal heat sterilization. The solution contains 50.0±2.5 mg/mL ketoprofen ethylenediamine as determined by a sensitive and specific HPLC method. Ampoules stored for 12 weeks at 5° C., 25° C./60% RH, 30° C./60% RH, 40° C./75% RH show no visible signs of crystallization or significant deviation in pH and osmolarity and contain 100%±5% original ketoprofen content as determined by stability indicating HPLC.

### Example 18

### Pharmaceutical Composition Comprising Tenoxicam and Ethylenediamine

Materials: 1) Water for Injection (WFI), USP, Q.S. to 1000 mL; 2) Tenoxicam 20 grams; 3) ethylenediamine 1.82 grams; 4) Nitrogen gas, NF. The preparation is manufactured, protected from light and under cover of nitrogen gas throughout, which is bubbled into 800 mL aliquot of WFI for 20 minutes. Ethylenediamine is added to the WFI with stirring and stirring is continued for 30 minutes. Tenoxicam is slowly added with vigorous stirring for 60 minutes at 30° C. The heat is removed and stirring is continued until room temperature is reached. The volume is adjusted to 1000 ml with the remainder of WFI and stirred for 30 minutes. The pH is further adjusted to bring the final pH between 7.0 and 7.5. Using a pressurized source of nitrogen gas, the solution is filtered through a 0.22 μm cartridge and collected in a suitable staging vessel protected from exposure to ultraviolet light. The solution is filled into Type 12-mL ampoule, with pre- and post-nitrogen gas flush. The sealed ampoules are subjected to terminal heat sterilization. The solution contains 20.0 mg±1.0 mg/mL tenoxicam ethylenediamine as determined by a sensitive and specific HPLC method. Ampoules stored for 12 weeks at 5oC, 25oC/60% RH, 30oC/60% RH, 40oC/75% RH show no visible signs of crystallization or significant deviation in pH and osmolarity and contain 100%±5% original tenoxicam content as determined by stability indicating HPLC.

### DEFINITIONS

“Drug,” “pharmacological agent,” “pharmaceutical agent,” “active agent,” and “agent” are used interchangeably and are intended to have their broadest interpretation as to any therapeutically active substance which is delivered to a living organism to produce a desired, usually beneficial effect. In general, this includes therapeutic agents in all of the major therapeutic areas, also including proteins, peptides, oligonucleotides, and carbohydrates as well as inorganic ions, such as calcium ion, lanthanum ion, potassium ion, magnesium ion, phosphate ion, and chloride ion.

“Pharmaceutically or therapeutically acceptable excipient or carrier” refers to a substance which does not interfere with the effectiveness or the biological activity of the active ingredients and which is not toxic to the hosts, which may be either humans or animals, to which it is administered. Pharmaceutically or therapeutically acceptable excipients or carriers are well known in the art.

The term “complex” as used herein means any physical combination of two or more discrete chemical compounds. A complex includes, but is not limited to, a salt, a physical mixture, a chelate, and the like. For example, as used herein, a complex comprising ketoprofen and piperazine includes, but is not limited to: a salt comprising ketoprofen and piperazine; a physical mixture comprising ketoprofen and piperazine; a solid containing ketoprofen and piperazine wherein the ketoprofen and piperazine are associated by hydrogen bonding; a solid containing ketoprofen and piperazine wherein the ketoprofen and piperazine are associated by hydrophobic bonding; a solid containing ketoprofen and piperazine wherein the ketoprofen and piperazine are associated by ionic bonding; a solid containing ketoprofen and piperazine wherein the ketoprofen and piperazine are associated by hydrogen, ionic, and or hydrophobic bonding; a co-crystal containing ketoprofen and piperazine. The complex can be constructed through several modes of molecular recognition including hydrogen-bonding, pi-stacking, guest-host complexation, and Van-Der-Waals interactions.

“Therapeutically effective amount” refers to the amount of an active agent sufficient to induce a desired biological result. That result may be alleviation of the signs, symptoms, or causes of a disease, or any other desired alteration of a biological system.

The phrase “therapeutically-effective” is intended to qualify the amount of each agent which will achieve the goal of improvement in disease severity and the frequency of incidence over treatment of each agent by itself, while avoiding adverse side effects typically associated with alternative therapies.

The term “effective amount” means the quantity of a compound according to the invention necessary to prevent, to cure, or at least partially arrest a symptom of local pain or discomfort in a subject. A subject is any animal, preferably any mammal, more preferably a human. Amounts effective for creating a substantially local therapeutic effect will, of course, depend on the severity of the disease causing the painful condition, and the weight and general state of the subject. Typically, animal models; such as those described in the Background and Examples herein, may be used to determine suitable dosages to be used. In addition, various general considerations taken into account in determining the “therapeutically effective amount” are known to those of skill in the art and are described, e.g., in Gilman et al., eds., Goodman And Gilman's The Pharmacological Basis of Therapeutics, 10th ed., McGraw Hill (2001); Remington: The Science and Practice of Pharmacy, 21st ed, Lippincott Williams & Wilkins (2005); and Martindale: The Complete Drug Reference, 34th Edition, Pharmaceutical Press (2004), each of which is herein incorporated by reference.

The term “parenteral” and “parenteral administration” herein encompasses administration of a composition by means other than through the gastrointestinal tract such as into or through the skin of a subject, and includes intradermal, subcutaneous, intramuscular, intravenous, intramedullary, intra-articular, intrasynovial, intraspinal, epidural, ocular, intrathecal and intracardiac administration. The term “parenteral” and “parenteral administration” herein also encompasses infiltration or topical application to a surgical site or open wound. Any known device or delivery system useful for parenteral injection or infusion of drugs can be used to effect such administration.

The term “subject” for purposes of treatment includes any animal subject who has any one of the known forms of pain. The subject is preferably a mammal and more preferably is a human. The subject can of course include other non-human animals, preferably horses, livestock, cattle, domesticated animals, cats, dogs, and the like.

All types of pain are contemplated by this invention, The term “pain”, also referred to as “analgesia” herein encompasses acute pain, subacute pain, chronic pain, cancer pain, breakthrough pain, neuropathic pain, nociceptive pain, visceral pain, idiopathic pain, inflammatory pain, non-inflammatory pain. Nonlimiting examples of acute pain include peri-operative pain, postsurgical pain, headache, acute low back pain, fractures, strains and sprains, ligament pain, cystitis, post-traumatic pain, burn pain, instrumentation pain, and renal colic. Nonlimiting examples of chronic pain include cancer pain, osteoarthritis, fibromyalgia, low back pain, idiopathic pain, rheumatoid arthritis, bursitis, myofascial pain and the like. Nonlimiting examples of neuropathic pain include postherpetic neuralgia, trigeminal neuralgia, painful diabetic neuropathy, pain HIV associated neuropathy, painful polyneuropathy, phantom limb pain, stump pain, spinal cord injury pain, post-stroke pain, central pain and the like.

As used herein, “equianalgesic doses,” also referred to as “analgesic equivalence,” is a term used by practitioners of the art to refer to approximately comparable doses of analgesics required to provide a similar magnitude of analgesia. There are established standards to allow practitioners of the art to convert the dose of one analgesic, given by any route of administration, to an approximately equivalent dose of another analgesic, given by any route of administration. These analgesic conversion tables provide what in the art is called “analgesic equivalence” or “equianalgesic doses” (Principles of Analgesic Use in the Treatment of Acute Pain and Cancer Pain, Fourth Edition, 5^{th} ed, American, Pain Society (2003); Gutstein H B & Akil H. Opioid Analgesics. In: Goodman and Gilman's The Pharmacologic Basis of Therapeutics, 10th Ed., Hardman J G & Limbird L E (Eds), p 569-619, McGraw-Hill, New York, N.Y.); Roberts L J & Morrow J D. Analgesic-Antipyretic and Antiinflammatory Agents and Drugs Employed in the Treatment of Gout. In: Goodman and Gilman's The Pharmacologic Basis of Therapeutics, 10th Ed., Hardman J G & Limbird L E (Eds), p 687-731, McGraw-Hill, New York, N.Y.). The availability of analgesic equivalence tables allows practitioners of the art to convert patients from one analgesic to another without a protracted titration period on the new analgesic.

Having now fully described the invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

1-53. (canceled)

54. A composition in a form suitable for parenteral administration comprising a complex comprising (a) an analgesic compound selected from the group consisting of ketoprofen, dexketoprofen, piroxicam, tenoxicam, and mixtures thereof; and (b) a compound selected from piperazine, ethylenediamine, and mixtures thereof; provided that, when said analgesic compound is piroxicam alone or tenoxicam alone, said compound is ethylenediamine; and provided that when the complex comprises ethylenediamine and piroxicam alone, the molar ratio of piroxicam to ethylenediamine is greater than 1:1.

55. The composition of claim 54, wherein the ratio of said analgesic compound to said compound selected from piperazine, ethylenediamine, and mixtures thereof is from about 10:1 to about 1:10.

56. The composition of claim 54, wherein the complex has an aqueous solubility of greater than 10 mg/mL.

57. The composition of claim 54, wherein the complex has an aqueous solubility of greater than 100 mg/mL.

58. The composition of claim 54, wherein said analgesic compound is ketoprofen or dexketoprofen, and said compound is ethylenediamine in a ratio of ketoprofen or dexketoprofen to ethylenediamine from about 1:3 to about 3:1.

59. The composition of claim 54, wherein said analgesic compound is piroxicam, said compound is ethylenediamine, and the ratio of said piroxicam to ethylenediamine from 1:0.999 to about 3:1.

60. The composition of claim 54, wherein said analgesic compound is tenoxicam, said compound is ethylenediamine, and the ratio of said tenoxicam to ethylenediamine is from about 1:3 to about 3:1.

61. The composition according to claim 54, wherein the injection volume of a single therapeutic dose does not exceed 5 mL.

62. The composition according to claim 61, wherein the said composition is substantially free of one or more of, or all of, the following: a surfactant, benzyl alcohol, a polyethylene glycol, a N-methylglucamine or derivative thereof, arginine or a derivative thereof, an adjuvant, citric acid or a derivative thereof, glycine or a derivative thereof, glycerol or a derivative thereof, and an alkylammonium compound.

63. The composition according to claim 61, further comprising an aqueous carrier and one or more pharmaceutically acceptable buffers.

64. The composition of claim 61, which is contained in a sealed syringe, vial or ampoule containing a sterile aqueous solution.

65. The composition of claim 61, further comprising sterile water; and optionally (a) one or more pharmaceutically acceptable buffers; and/or (b) one or more pharmaceutically acceptable preservatives; and wherein the pH is from about 6.5 to about 8.5; and the concentration of said analgesic compound is from about 0.1 mg/mL to about 100 mg/mL.

66. The sealed syringe, vial or ampoule according to claim 64, wherein said aqueous solution further comprises one or more pharmaceutically acceptable excipients, optionally contains one or more pharmaceutically acceptable buffers, and the pH of said solution is from about 6.5 to about 8.5.

67. A kit for use in treating or preventing the acute pain, comprising: (i) a dosage form comprising the pharmaceutical composition of claim 61 in an ampoule, vial, prefilled syringe, or autoinjector; (ii) a container for the dosage form; and (iii) optionally, alcohol swabs.

68. A method of treating or preventing pain, inflammation, and/or fever in a subject in need of such treatment, said method comprising parenterally administering to a human in need of such treatment a pharmaceutical composition comprising an analgesic compound selected from the group consisting of ketoprofen, dexketoprofen, tenoxicam, piroxicam, and mixtures thereof and a compound selected from piperazine, ethylenediamine, and mixtures thereof; provided that, when said analgesic compound is tenoxicam alone, said compound is ethylenediamine, and provided that when the complex comprises ethylenediamine and piroxicam alone, the molar ratio of piroxicam to ethylenediamine is greater than 1:1.

69. The method of claim 68, wherein improved local tolerability, improved venous tolerability, reduced local irritation, reduced injection site pain, and/or reduced phlebitis is provided, as compared to administration of said analgesic compound without said piperazine or ethylenediamine.

70. The method of claim 68, wherein the pharmaceutical composition is administered intravenously, intramuscularly, intrathecally, epidurally, ocularly, or subcutaneously.

71. The method of claim 68, wherein said analgesic compound is ketoprofen or dexketoprofen and said compound is ethylenediamine.

72. The method of claim 68, wherein said analgesic compound is tenoxicam and said compound is ethylenediamine.

73. The method of claim 68, wherein said pain is an acute pain selected from postsurgical pain, acute posttraumatic pain, acute renal colic pain, acute migraine pain, burn pain, breakthrough pain and burn dressing change pain.

74. The method of claim 68, wherein said fever is postsurgical fever.

75. The method of claim 68, wherein said pain is cancer pain.

76. A method for the treatment of patent ductus arteriosus or intraventricular hemorrhage in an infant in need of such treatment of treating or preventing pain, inflammation, and/or fever in a subject in need of such treatment, comprising parenterally administering a pharmaceutical composition comprising (i) an analgesic compound selected from the group consisting of ketoprofen, dexketoprofen, tenoxicam, piroxicam and mixtures thereof and (ii) a compound selected from piperazine, ethylenediamine, and mixtures thereof; provided that, when said analgesic compound is tenoxicam alone, said compound is ethylenediamine, and provided that when the complex comprises ethylenediamine and piroxicam alone, the molar ratio of piroxicam to ethylenediamine is greater than 1:1.

77. An in situ process for preparing a parenteral analgesic composition, comprising adding one or more of an analgesic compound selected from the group consisting of ketoprofen, dexketoprofen, piroxicam, tenoxicam, and mixtures thereof in racemic, enantiomeric excess, or enantiomeric form to an aqueous solution comprising a compound selected from the group consisting of ethylenediamine, piperazine, and mixtures thereof; and mixing until a complex is formed in solution; provided that, when said analgesic compound is tenoxicam alone or piroxicam alone, said compound is ethylenediamine; and provided that when the complex comprises ethylenediamine and piroxicam alone, the molar ratio of piroxicam to ethylenediamine is greater than 1:1.
